(19) United States
(12) Patent Application Publication
(10) Pub. No.: US 2011/0111012 A1
(43) Pub. Date: May 12, 2011

(54) Nanomaterial wound dressing assembly

(75) Inventors: Clinton B. Pepper, West Linn, OR (US); Simon McCarthy, Portland, OR (US); Keith Real, Delgany (IE); Jess Kimball, Portland, OR (US); Jana Ruzickova, Vratislavica nad Nisou (CZ); Jana Svobodova, Slate Pavlovica (CZ); Lucia Silovska, Vratislavica nad Nisou (CZ); Jitka Vankova, Brno-Slalina (CZ); Jiri Machat, Reckovica (CZ); Radim Dvorak, Tisnov (CZ)

(73) Assignee: HEMCON MEDICAL TECHNOLOGIES, INC.

(21) Appl. No.: 12/590,712

(22) Filed: Nov. 12, 2009

(51) Int. Cl. A61K 9/70; A61K 33/38; A61K 38/16; A61K 31/715; A61K 31/722; A61K 31/717; A61P 19/02; B32B 37/02; B29C 47/00

(52) U.S. Cl. 424/445; 424/618; 514/54; 514/55; 514/57; 156/60; 156/244.17; 156/64; 977/887; 514/17.2

(57) ABSTRACT

Wound dressing assemblies and methods of producing the wound dressing assemblies. The wound dressing assemblies various comprise individual layers of nanomaterials that have been formed according to an electrospinning process.

Each of the layers can have different characteristics and qualities. The invention also provides guidance in selecting materials to form a wound dressing assembly, as well as possible adaptations to the electrospinning process to provide different characteristics for particular materials.

### BACKGROUND OF THE INVENTION

The present invention relates to wound dressing assemblies prepared from a needleless electrospinning process that comprise single or various, individual layers of material(s). More specifically, the present invention is directed towards wound dressing assemblies that comprise single or various layers of electrospun material(s).

Electrostatic spinning, or electrospinning, has been dependent on spinning of cohesive liquids across a high voltage electric field through hollow needles or capillaries. More recently needleless electrospinning methods (where highly cohesive liquid jets are spontaneously created from liquid surfaces) have enabled improved scaling of the spinning of nanofiber web and sheet. An important example of the needleless spinning approach is where one electrode of a high voltage electric field is a wetted rotating cylinder from whose surface jets of cohesive liquid are propelled by the electric field in the direction of a counter electrode. The spinning electrode forms nanofibers from a polymer solution. The opposite, parallel pole of the voltage source acts as a collecting electrode, wherein the formed nanofibers are propelled towards the collecting electrode and are deposited on a substrate material. The deposited fibers form a linear web of materials upon the substrate. This needleless rotating cylinder approach to the formation of nanofibers provides an important advantage over the capillary or needle approach in that innumerable jets can be formed at one time and hundreds to thousands of grams of nanofibers can be deposited per hour.

Developments in electrospinning processes have generally focused on particular materials that are capable of being spun and research into new materials or compositions that may be spun. Research is still advancing as to the properties and characteristics of these nanomaterials, as well as research into possible uses of these materials.

### SUMMARY OF THE INVENTION

The present invention provides various nanospun materials and compositions of materials, as well as methods for producing the nanospun materials.

The nanospun materials are incorporated into wound dressing assemblies, wherein the wound dressing assemblies consist of single or multiple layers of nanospun materials. The individual nanospun material layers may each comprise different materials and compositions, with the individual layers being joined or adhered together to form a dressing assembly. Because the individual layers are potentially spun from different materials and compositions, the dressing assembly can be formed and designed to address particular needs, based on a variety of factors, such as hemostasis, fluid handling, drug delivery, antimicrobial and/or other. The present invention also contemplates methods of determining particular materials for each layer of the dressing assembly based on the requirements of the final wound dressing assembly.

The present invention further contemplates electrospinning methods for altering the characteristics of the nanomaterials that are being spun.

The present invention also contemplates various compositions for use as nanospun materials, including various materials that have been cross-linked with each other.

### BRIEF DESCRIPTION OF THE DRAWINGS

FIG. 1 is a perspective view of a wound dressing assembly according to the present having various layers on nanospun material.

FIG. 2 is a cross-sectional view of the wound dressing assembly shown in claim 1.

FIG. 3 is a perspective view of the dressing assembly shown in FIG. 1 being applied to a wound site.

FIG. 4 is a perspective view of the wound dressing assembly shown in FIG. 3, demonstrating fluids being absorbed within the dressing assembly.

FIG. 5 depicts the top layer of the dressing assembly of FIG. 4 being removed after the dressing assembly has been applied to the wound dressing for an adequate period of time.

FIG. 6 is a schematic diagram of a continuous needleless electrospinning process carried out on a machine manufactured by Elmarco s.r.o, located in the Czech Republic, with one rotating spinning electrode for producing nanomaterials for use in the present invention.

FIGS. 7A and 7B demonstrate an alternate rotating spinning electrode used in the process shown in FIG. 6.

FIGS. 8A and 8B demonstrate another alternate electrode for use in an electrospinning process as shown in FIG. 6.

FIGS. 9A and 9B demonstrate yet another alternate electrode for use in an electrospinning process as shown in FIG. 6.

FIGS. 10A and 10B demonstrate a wire spinning electrode for use in an electrospinning process as shown in FIG. 6.

FIG. 11 is a schematic diagram of a NanoSpider electrospinning process that utilizes a needle collection device during the process, which is a different process than the “needleless” processes associated with electrospinning wherein a fiber-jet is emitted from a rotating spinning electrode surface and not emitted from a hollow needle (i.e. “needleless).

FIG. 12 is a schematic diagram of a continuous needleless electrospinning process with four rotating spinning electrodes, with a single material being processed.

FIG. 13 is a schematic diagram of a continuous needleless electrospinning process with four rotating spinning electrodes, with four different materials being processed.

FIG. 14A is a schematic diagram of a continuous needleless electrospinning process with three rotating spinning electrodes, processing three different materials being processed, including an adhesive layer.

FIG. 14B is a schematic diagram as shown in FIG. 14B, with an alternate process of providing an adhesive layer.

FIG. 15 is an exploded view of the wound dressing assembly shown in FIG. 1, prior to the layers being compressed.

FIG. 16 is a schematic of the layers in FIG. 15 being compressed to one another using a rotary press.

FIG. 17 is a schematic of the layers in FIG. 16 being cut into sizes for individual dressing assemblies

FIG. 18 provides a perspective view of an alternate wound dressing assembly according to the present invention, which includes an internal layer of a powder form of an electrospun nanomaterial.

FIG. 19 is, a cross-sectional view of the wound dressing assembly shown in FIG. 18.

FIG. 20 demonstrates a step in forming the wound dressing assembly of FIG. 18, with separate layers of the wound dressing assembly being adhered to one another to form an internal cavity between the layers.

FIG. 21 demonstrates a further step in forming the wound dressing assembly of FIG. 20, wherein a hemostatic and/or therapeutic material is inserted into the cavity shown in FIG. 8.

FIG. 22 shows a further step in forming a wound dressing assembly of FIG. 20, wherein the material described in FIG. 21 is sealed within the formed cavity.

FIG. 23 is a diagram depicting a process for selecting materials and process conditions for a wound dressing assembly according to the present invention.

FIG. 24 is a further diagram depicting range of treatment options for a wound dressing assembly according to the present invention.

FIG. 25 is a diagram depicting possible material attributes for a wound dressing assembly according to the present invention.

FIG. 26 is a diagram depicting possible materials and their properties for a wound dressing assembly according to the present invention.

FIGS. 27A-27C are tables depicting characteristics of various materials that can be electrospun to form a nanomaterial and that can be used in a layered wound dressing assembly.

FIG. 28 provides scanning electronic image (SEM) photographs of a needleless electrospun layer of a chitosan/polyethylene oxide (PEO) matrix, at magnifications of 1000× and 20000×.

FIG. 29 provides SEM photographs of chitosan/PEO matrices having various densities.

FIG. 30 provides a graphical comparison between the individual fiber diameter and the density of electrospun chitosan/PEO matrices.

FIG. 31 provide examples of various compounds that can be used to carry out cross-linking of various compositions and materials in the present invention, including chitosan and polyethylene oxide (PEO) shown in FIG. 21.

FIG. 32 provides SEM photographs of an electrospun cross-linked chitosan/PEO matrix.

FIG. 33 provides a table of samples of electrospun thermally treated chitosan/PEO materials.

FIG. 34 provides graphical representations of the antimicrobial effects of electrospun non-thermally treated chitosan/PEO matrices on various microorganisms (compliance is with USP <28>).

FIG. 35 provides SEM photographs of the non-thermally treated chitosan/PEO matrices of discussed in FIG. 34.

FIG. 36 provides graphical representations of the antimicrobial effects of electrospun thermally treated chitosan/PEO matrices on various microorganisms (compliance is with USP <28>).

FIG. 37 provides SEM photographs of the thermally treated chitosan/PEO matrices of discussed in FIG. 36.

FIG. 38 provides antimicrobial effects and SEM photographs of non-thermally treated chitosan/PEO+silver matrices (compliance is with USP <28>).

FIG. 39 provides antimicrobial effects and SEM photographs of thermally treated chitosan/PEO matrices containing silver nanoparticles (compliance is with USP <28>).

FIG. 40 graphically compares nanofiber diameter and basis weights between thermally treated chitosan/PEO matrices with and without silver nanoparticles incorporated into the matrices.

FIG. 41 provides SEM photographs of electrospun chitosan/PEO matrices prepared from an aqueous lactic acid.

FIG. 42 provides SEM photographs of electrospun chitosan/PEO matrices which have been prepared from a solution containing both acetic and lactic acids.

FIG. 43 provides graphical representations of the antimicrobial effects of electrospun thermally treated chitosan/PEO matrices shown in FIG. 42 on various microorganisms (compliance is with USP <28>).

FIG. 44 provides graphical representations and comparison of the antimicrobial effects of various electrospun chitosan/PEO matrices on various microorganisms (compliance is with USP <28>).

FIG. 45 is a graphical representation of electrospun chitosan materials, comparing the absorption capacity with relative humidity of the electrospinning conditions.

FIG. 46 is a graphical representation of electrospun chitosan materials, comparing the absorption capacity with basis weight of the electrospun materials with an electrospinning relative humidity being 30% and a 3-day old solution.

FIG. 47 is a graphical representation of electrospun chitosan materials, comparing the absorption capacity with basis weight of the electrospun materials with an electrospinning relative humidity being 40% and a 3-day old solution.

FIG. 48 is a graphical representation of electrospun chitosan materials, comparing the absorption capacity with basis weight of the electrospun materials with an electrospinning relative humidity being 20% and a 10-day old solution.

FIG. 49 is a graphical representation of electrospun chitosan materials, comparing the absorption capacity with basis weight of the electrospun materials with an electrospinning relative humidity being 30% and a 10-day old solution.

FIG. 50 is a graphical representation of electrospun chitosan materials, comparing the absorption capacity with basis weight of the electrospun materials with an electrospinning relative humidity being 20% and a 40-day old solution.

FIG. 51 depicts the effects of heating on gel permeatation chromatography molecular weight elution profile of a chitosan electrospinning process solution.

FIG. 52 depicts the effects of stirring on the gel permeatation chromatography molecular weight elution profile of a chitosan electrospinning solution.

FIG. 53 is a graph comparing change in viscosity over time of an aqueous acetic acid chitosan solution with chitosan/PEO solutions.

FIG. 54 depicts gel permeatation molecular weight elution profiles of chitosan solutions (with polyethylene oxide) aged for 5, 22.5, 42, and 65.5 hours, and 37 days.

FIG. 55 depicts gel permeatation chromatography molecular weight elution profiles of chitosan solutions (with polyethylene oxide) aged for 5, 22.5, 42, and 65.5 hours, and 37 days.

FIG. 56 is a table of polyvinyl alcohol (PVA)/microdispersed oxidized cellulose (MDOC) samples that have been subjected to a needleless electrospinning process.

FIGS. 57-75 are graphical representations comparing various qualities and characteristics of the electrospun PVA/MDOC materials listed in FIG. 55.

FIG. 76 are SEM images at various magnifications of electrospun MDOC/PVA solutions having a 3:7 ratio.

FIG. 77 are SEM images at various magnifications of electrospun MDOC/PVA solutions having a 4:6 ratio.

FIG. 78 are SEM images at various magnifications of electrospun MDOC/PVA solutions having a 5:5 ratio.

FIG. 79 are SEM images at various magnifications of electrospun MDOC/PVA solutions having a 6:4 ratio.

FIG. 80 is a graphical representation comparing the effects of conductivity on the area weight of an electrospun PVA material.

FIG. 81 is a table depicting the relative production capacities of the electrospun PVA/MDOC materials listed in FIG. 56.

FIG. 82 presents a graphical representation comparing the whole blood clotting time percentages of various electrospun and nonspun matrices as listed in FIG. 83.

FIG. 83 is a table comparing the whole blood clotting time percentages of various electrospun and nonspun matrices.

FIG. 84 presents a graphical representation comparing the whole blood clotting time percentages of various electrospun chitosan/PEO matrices as listed in FIG. 59.

FIG. 85 is a table comparing the whole blood clotting time percentages of various electrospun chitosan/PEO matrices.

FIG. 86 is a table comparing contact activation enzymes (Factor XII and kallikrein) of certain electrospun and nonelectrospun materials.

FIG. 87 is a graphical representation comparing intrinsic pathways of activated partial thromboplastin times (APTTs) of the materials depicted in FIG. 86.

FIG. 88 is a table comparing clotting times (APTTs) of the materials depicted in FIG. 86.

FIG. 89 is a graphical representation comparing extrinsic pathways of activated prothrombin times (PTs) of the materials depicted in FIG. 86.

FIG. 90 is table comparing clotting times (PTs) of the materials depicted in FIG. 89.

FIG. 91 is a graphical representation comparing the aggregation of platelet rich plasma (PRP) of the materials depicted in FIGS. 86-90.

FIG. 92 is a table comparing the maximum aggregation of PRP shown in FIG. 91 of the various materials.

FIG. 93 is a table comparing the whole blood clotting times of various electrospun materials, as well as comparison to nonspun chitosan materials.

FIG. 94 is a graphical representation comparing absorption capacities of electrospun chitosan/PEO matrices under various electrospinning conditions.

FIG. 95 is a graphical representation comparing the basis weight (g/m²) of an electrospun chitosan/PEO matrix with and without an air stream being used during the electrospinning process.

FIG. 96 is a table showing mean fiber diameters of various samples of electrospun materials and mean fiber diameters of the electrospun materials, with the electrospinning process being carried out at various voltages.

FIG. 97 is a graphical representation comparing the absorption capacity of electrospun materials with different voltages being applied during the electrospinning process.

FIG. 98 is a table comparing mean fiber diameters of electrospun materials, with different voltages being applied during the electrospinning process.

FIG. 99 is a table comparing mean fiber diameters and viscosities of electrospun materials with different voltages being applied during the electrospinning process.

FIG. 100 provides a graphical representation, monitoring the current at various times during an electrospinning process.

FIG. 101 provides a graphical comparison of the change in temperature with time of a spinning solution used for a needleless electrospinning process, with and without the electrode spinning.

FIG. 102 is a graphical representation showing the change of spinning solution viscosity with time during a needleless electrospinning process.

FIG. 103 provides a graphical representation, monitoring the current at various times during an alternate electrospinning process from that shown in FIG. 100.

FIG. 104 provides a table comparing basis weights of various electrospun materials carried out according to the process depicted in FIG. 103.

FIG. 105 provides a table comparing changes in mean fiber diameters during the process depicted in FIG. 103.

FIG. 106 is a graphical representation showing the effect of temperature on viscosity during the electrospinning process for solutions comprising MDOC/PVA.

FIG. 107 is a graphical representation comparing the viscosity to the expected dry matter content of an electrospun MDOC/PVA solution during an electrospinning process.

FIG. 108 is a graphical representation comparing the relative viscosity to the expected dry matter during an electrospinning process for MDOC/PVA compositions.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

The present invention contemplates various wound dressing assemblies comprised of nanomaterials and methods of making the wound dressing assemblies. Generally speaking the wound dressing assemblies comprise single or various individual layers of nanomaterial(s) that are combined to form the wound dressing assembly. Depending on the particular uses and requirement for an individual wound dressing assembly, the present invention provides methods for altering and adjusting the electrospinning parameters, as well as methods for determining which compounds and compositions should be incorporated into specific nanomaterial layers.
I. Process Overview for Producing an Electrospun Wound Dressing Assembly
II. Process Overview For Determining The Materials That Comprise the Wound Dressing Assembly
III. Various Material Characteristics
   A. General Example of Electrospun Materials
   B. Electrospun Chitosan/Polyethylene Oxide (PEO) Matrix
      1. Matrix Overview
      2. Cross-Linking of the Chitosan and Polyethylene Oxide
      3. Enhancement of the Chitosan/PEO Matrix For Increased Antimicrobial Activity
         a. Addition of Silver to the Chitosan/PEO Matrix
         b. Use of Alternative Acids in Production the Chitosan/PEO Matrix
      4. Influence of Relative Humidity on Chitosan/PEO Solution
      5. Effects of the Age of Chitosan/PEO Solution
         a. Mixing Speeds
         b. Temperature Increase
         c. Comparison of Chitosan Solution To Chitosan/PEO Solution
      6. Electrospun Chitosan Materials Without PEO
   C. Electrospun Microdispersed Oxidized Cellulose (MDOC)/Polyvinyl alcohol (PVA) Matrix
      1. Influence of Phosphoric Acid on the Strength and Elongation on an Insoluble MDOC/PVA Matrix (8:2 Ratio)
      2. Influence of Phosphoric Acid and Glyoxal on the Strength and Elongation of an Insoluble MDOC/PVA Matrix (3:2 Ratio)
      3. Influence of Phosphoric Acid on Strength and Elongation of a Soluble MDOC/PVA Matrix (8:2 Ratio)
      4. Influence of Phosphoric Acid and Glyoxal on Strength of a Soluble MDOC/PVA Matrix (3:2 Ratio)
      5. Influence of Phosphoric Acid on Strength and Elongation of a Soluble MDOC/PVA Matrix (8:2 Ratio)
      6. Effect of the Type of MDOC on the Mechanical Properties of the Matrix
         a. PVA/MDOC Combined With Various Acids
         b. Effect of Citric Acid Concentration on Crosslinking With PVA
         c. PVA Cross-Linking With MDOC-H+
      7. Effect of the Type of MDOC on The Mechanical Properties of The Matrix
      8. Effect of the Ratio of MDOC/PVA on The Mechanical Properties of the Matrix
      9. Optimization of MDOC/PVA Ratio
      10. Production Capacity
      11. The Effect of Solution Conductivity on PVA Electrospinning
      12. MDOC/PVA/PEO Matrix Further Comprising Chlorhexidine
   D. Gelatin Materials
      1. Combinations of Gelatin With Various Polymers
      2. Optimization of Gelatin Solution With PEO
   E. Electrospun Hyaluronic Solutions
   F. Electrospun CMC/PVA/PEO Solutions With Silver Nanoparticles
   G. Comparison of Hemostatic Qualities on Various Electrospun Materials
      1. Comparison of Hemostatic Qualities of Electrospun Materials
      2. Comparison of Electrospun Wound Dressing Assemblies to Other Types of Wound Dressing Assemblies
IV. Electrospinning Parameters
   A. Use of an Air Stream During the Electrospinning Process
   B. Change of Voltage During the Electrospinning Process
   C. Different Electrospinning Methods
   D. Interaction of Nanofibers and Collecting Substrates
      1. PA6/12+Chitosan−without Adhesive Layer
      2. PA6/12+Chitosan−with Adhesive Layer
      3. PA6=Chitosan−without Adhesive Layer
      4. PA6+Chitosan—with Adhesive Layer
V. Conclusion

### I. PROCESS OVERVIEW FOR PRODUCING AN ELECTROSPUN WOUND DRESSING ASSEMBLY

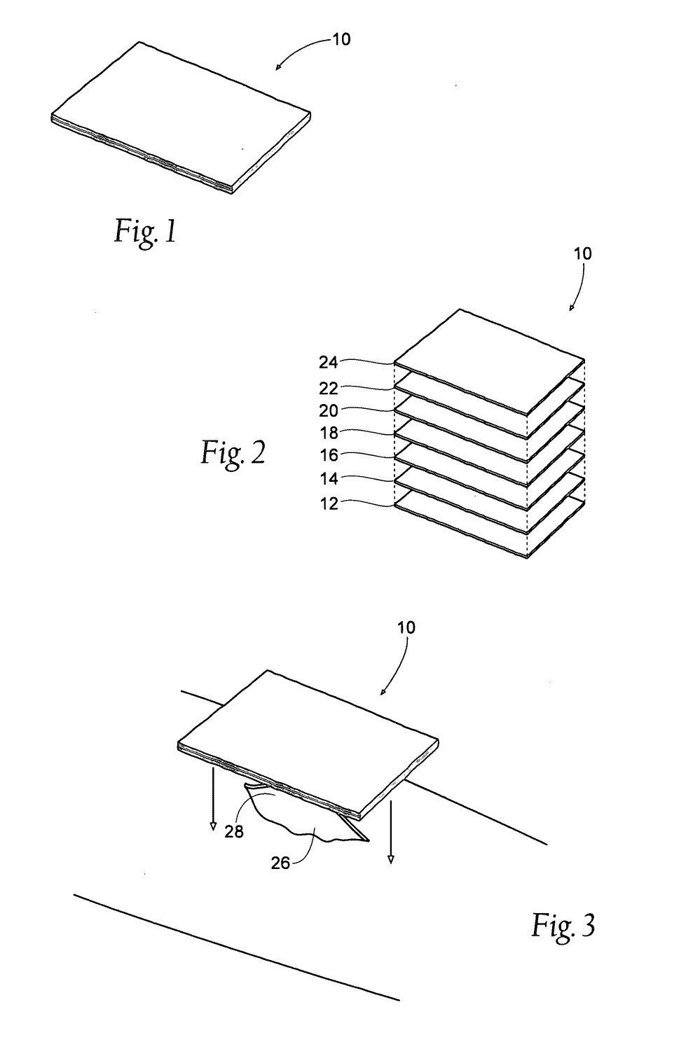

FIGS. 1 and 2 depict a wound dressing assembly 10 that has been developed and assembled according to the present invention. The wound dressing assembly 10 is formed of various individual layers of material, as is demonstrated in FIG. 2. Generally, the individual layers will consist of electrospun layers of nanomaterials, with the layers being secured or compressed together to form the wound dressing assembly 10. The electrospinning process for producing the individual layers of the assembly 10 will be discussed further with respect to FIGS. 6-14.

Still referring to FIGS. 1 and 2, the wound dressing assembly 10 comprises a plurality of layers. A first layer 12 comprises a microdispersed oxidized cellulose (M-DOC) material that has been crosslinked with a polyvinyl alcohol (PVA) material that has been further treated with silver particles. A second layer 14 consists of a gelatin material. Above the gelatin layer is a layer 16 of carboxymethyl cellulose (CMC) crosslinked with a polyethylene oxide (PEO) material and a PVA material. The next layer 18 consists of a chitosan material treated with a PEO material. Another layer 20 of CMC material as described for layer 16 is above the layer 18, with a layer 22 consisting of a chitosan material as described for the layer 18 being arranged above the layer 20. The final layer 26 comprises a backing material having a high moisture vapor transmission rate (MVTR), which preferably provides a material that will allow a user to grab and manipulate the wound dressing assembly 10.

As opposed to the other layers of material that comprise the dressing assembly 10, the backing layer 26 is not comprised of an electrospun material. As such it should be understood that wound dressing assemblies developed according to the present invention may comprise one or more layers of electrospun material and also one or more layers of materials that comprises a material or materials that are not electrospun. The layers and materials will then be compressed to form the wound dressing assembly 10 as shown in FIG. 1. Alternately, it is possible that one or more of the layers of the material can be formed together during the electrospinning process. Processing of the layers will be discussed further with respect to FIGS. 12-17.

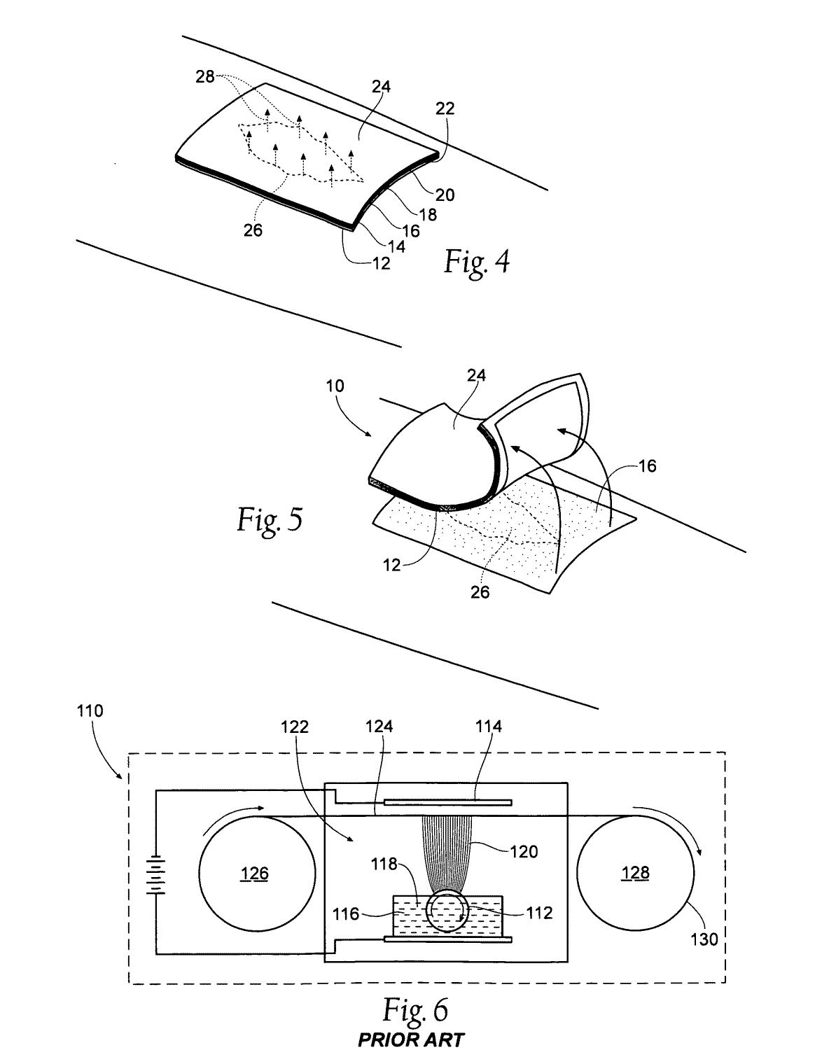

FIG. 3 demonstrates the layered dressing assembly 10 being applied to a wound 26. The layered dressing assembly 10 is applied with the first layer 12 being applied directly onto the open wound 26, which interacts with the blood 28 of the wound 26. The MDOC nanomaterial of the first layer 12 will quickly dissolve, allowing the blood 20 to interact with the interior layers 14, 16, 18, 20, and 22, as depicted in FIG. 4. As the blood 20 moves through the first layer 12 into the interior layers, the interior layers will begin to interact directly with the blood 20, thereby providing therapeutic and antiseptic treatment for the wound 18. For example the layers 18 and 22, i.e. the chitosan layers, are generally insoluble in water and will provide a barrier that will prevent the blood 28 from flowing outwardly from the dressing assembly 10, thereby contributing to the ability of the dressing assembly 10 to treat the wound 26. Also, the gelatin layer 14 can also assist in coagulating the blood 28 on the open wound.

After the dressing assembly 10 has been applied to the wound 26 for a sufficiently determined time, the backing layer 24 may be removed from the wound 18, as depicted in FIG. 5. The first layer 12 comprising the MDOC material has dissolved, as previously discussed, and the interior layers 16, 18, 20, and 22 have interacted with the wound 26 and the blood 28 to begin to promote hemostasis and wound healing. The interior layers have also formed a sufficient barrier between the wound 26 and the external environment. Thus, the backing layer 24 can be removed from the wound 26, while the interior layers remains to further promote hemostasis. Alternatively, the backing layer 24 could remain on the wound 26, which could potentially further promote anti-microbial activity.

The depicted layered dressing assembly 10 provides an assembly and system that provides different materials that have different therapeutic qualities. The assembly 10 comprises different layers of materials joined together in a manner directed by the user, determinative on the specific use required for a layered dressing assembly. As noted, the assembly 10 comprises layers of different materials, with the possibility that one or more of the layers will be of the same material.

Each of the separate layers comprises a separate layer of nanomaterial that can be individually woven and formed, determinative of the specific needs for the dressing assembly. FIGS. 6-17 provide an overview of how the dressing assembly 10 is formed.

Needleless electrospinning, as described according to the process described for the present invention, is an efficient way of producing nanofibers of various polymers and polymer blends to form composite fibers. Another advantage of this method is the possibility of adding small insoluble particles to the polymer solution which are subsequently encapsulated in the dry fibers. This enables the addition of soluble drugs or antimicrobial agents to the polymer textiles of nanofibers. In the present invention, the use of nanofibers allows for the delivery of therapeutic material, hemostatic or medicinal materials to a wound site. Further, the present invention provides electrospinning of individual layers of various materials or compositions that can be joined together after the individual layers have been formed with the spinning process.

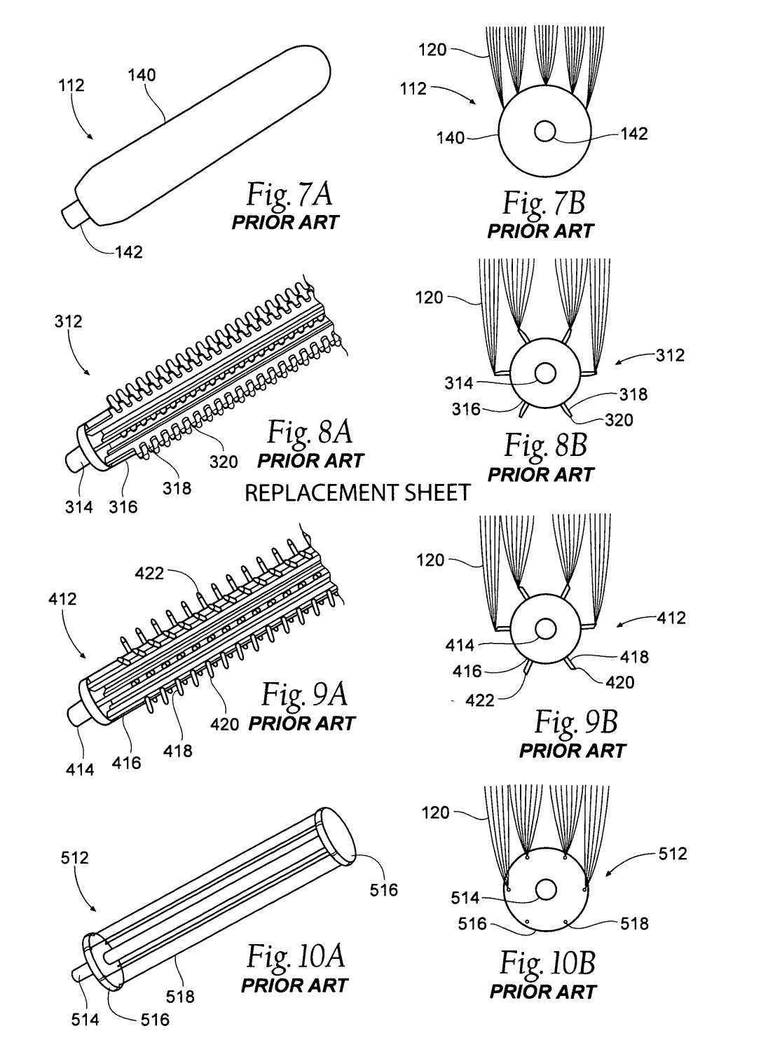

FIG. 6 demonstrates a needleless device 100 used for an electrostatic spinning, or electrospinning, process for the production of nanofibers. Elmarco in the Czech Republic manufactures needleless electrospinning machines with one spinning electrode (such as depicted in FIG. 6) or with multiple spinning electrodes such as depicted in later FIGS. 7A-10B. The Elmarco needleless electrospinning machine family is covered by the trademarked name NanoSpider. Proprietary spinning electrodes typically used in NanoSpider electrospinning are depicted in FIGS. 7A to 10B. The electrospinning process has a rotary spinning electrode 112 and a parallel positioned collecting electrode 114, which are connected in a circuit. As shown in FIG. 6, the spinning electrode 112 has an elongated cylindrical body. However, other shapes for the electrode are possible, as depicted in FIGS. 7A-10B. The spinning electrode 112 is mounted in a reservoir 116 of a polymer solution 118.

The polymer solution 118 provides the source of the formed nanofibers 120, which can be one of any number of various types of materials. The spinning electrode 112 is connected to a pole of a high voltage source. For example, the spinning electrode may be connected to the positive pole. The collecting electrode 114 may either be grounded or connected to the opposite pole of that of which the spinning electrode 112 is connected (i.e. the negative pole). The collecting electrode 114 may be formed by a grounded wire, or a rod, or another known arrangement that can act as an electrode. An induced electrostatic field of high intensity is formed between the electrodes 112 and 114, which creates a spinning space 122 for the production of the nanofibers 120. Still referring to FIG. 6, a continuous web of material 124 is located upon a first spindle 126. The web of material 124 will pass through the spinning space 122 and exit onto a second rotating spindle 128, which will provide the necessary movement for the web of material 124 to pass through the device 100. As the material 120 passes through the spinning space 122, the nanofibers 120 are deposited upon the web 124, thereby forming a nanofiber mat 130. Depending on the speed as to which the web 124 passes through the spinning space 122, the voltage across the electric field, and the speed that the spinning electrode 112 spins, the thickness of the mat 130, i.e. the amount of nanofibers 120 deposited on the web of material 124, can be adjusted. The diameter of fibers are generally in the range of 100-500 nm and the basis weight of the nanofibrous web prepared can be in the general range of 0.01-100 g/m².

Likewise, by altering the parameters of the electrospinning arrangement, it is possible to alter the positioning of the individual nanofibers 120 as they are deposited on the mat 130. For example, the nanofibers 120 can be deposited in a relatively planar or flat fashion, which is generally stated as being a two-dimensional arrangement (2-D), or the materials can be placed on the mat 130 in a less planar fashion, referred to as a “fluffy” arrangement. The fluffy arrangement can be considered an arrangement wherein the layers are positioned in a three-dimensional (3-D) arrangement. The “fluffy” 3-D arrangement shows potential as having possible advantages by providing more of a volume than a 2-D, such as for increased absorption potential.

The above is exemplary of possible techniques that can be used according to the present invention. Examples of other electrospinning processes can be found in U.S. applications, Pub. Nos. 2008/0284050 and 2008/0307766, which are herein incorporated by reference. Likewise, the electrospinning process could be carried out by using a melt spinning process.

As previously noted, the spinning electrode 112 comprises a cylinder 140, as shown in FIGS. 7A and 7B. The cylinder 140, which is formed along a central axis 142, creates the spinning electrode 112 for use as shown in FIG. 6.

Other shapes and forms for the spinning electrode 112 are shown in FIGS. 8A-10B. FIGS. 8A and 8B provide an alternate electrode 312 for use in the electrospinning process. The electrode 312 generally comprises a central shaft 314 with a plurality of faces 316 that are attached to and extend outwardly from the shaft 314. The faces 316 each have a plurality of lamellas 318 extending off of the faces 316. Each of the lamellas ends in a tip 320, with the nanofibers 120 moving off of the tips towards the electrode 114 (see FIG. 6).

FIGS. 9A and 9B provide another alternate electrode 412 that is similarly arranged to the electrode 312. The electrode 412 has a central shaft 414 with a plurality of faces 416 that are attached to and extend outwardly from the shaft 414. The faces 416 each have a plurality of lamellas 418, as well, that end in a tip 420. The tips 420 differ from the tips 320 in that they end in a beveled surface 422, with the nanofibers moving from the beveled surface 422 towards the electrode (FIG. 6). The electrode 412 is commonly referred to as a “thorn” style electrode.

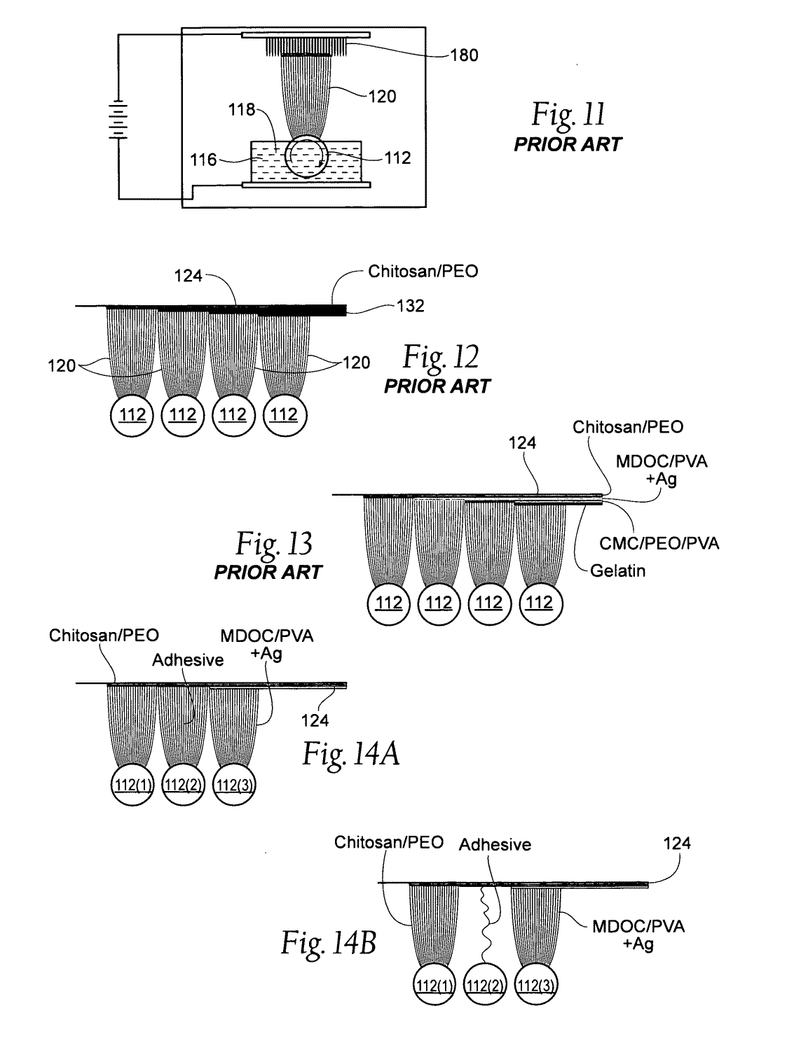

FIGS. 10A and 10B show an electrode 512 having a central shaft 514 supporting a pair of circular members 516. A plurality of wires 518 are mounted between the circular members 516. As the shaft 514 rotates, the electrical charge created will allow the nanofibers 120 to move from the wires 518 towards the electrode 114 (FIG. 6).

Use of the various electrodes will be discussed further in the following sections.

The depicted electrospinning process is considered a “needleless” electrospinning process, which is generally the preferred process of producing wound dressing assemblies according to the present invention. As stated above, it is possible to have a different type of needle for use in collection as shown in FIG. 11. The shown process has a plurality of needles 180 that assist in the collection of the electrospun material 120. These collecting needle arrays are different from the needleless (nozzle-less) process associated with electrospinning where a fiber-jet spontaneously is emitted from the rotating spinning electrode surface and is not emitted from a hollow needle (i.e. “needleless”), which has been the accepted process of electrospinning until this decade.

As mentioned above, the various nanofiber mats 130 can be formed of an individual material or a composition of materials. FIGS. 12-14B depict various arrangements of nanofiber mats. Each of the figures demonstrates an electrospinning process that utilizes multiple electrodes 112 to produce alternative layered arrangements of the nanofiber mats 130.

FIG. 12 depicts four individual electrodes 112 for spinning a single type of solution to form a nanomaterial. That is, each of the four electrodes 112 forms the same nanomaterial 120 that is deposited on the web 124 to form a layer of material 132. As an example, the solution for each electrode 112 is a chitosan/PEO solution. FIG. 12 demonstrates that the electrospinning process of the present invention can be utilized to alter the thickness of an individual layer during the electrospinning process.

FIG. 13 also depicts an electrospinning process using four individual electrodes 112. However, instead of each electrode 112 spinning the same type of solution, each electrode spins a different type of solution to form a nanomaterial 120. As an example, the four solutions could be a chitosan/PEO solution, an M-DOC/PVA with Ag solution, a CMC/PEO/PVA solution, and a gelatin solution, as described in the dressing assembly 10. The four solutions will be deposited in order on the web 124 to form four separate layers from a single electrospinning process.

As discussed previously, the individual layers may be adhered to one another, which may occur during or after the electrospinning process. FIGS. 14A and 14B show two examples of adhesives being applied to electrospun materials during the spinning process. In FIG. 14B, a layer of chitosan/PEO is spun from a first electrode 112(1), followed by a layer of adhesive form a second electrode 112(2), and then followed by a MDOC/PVA material from a third electrode 112(3). The resultant electrospun materials are deposited on the web 124, with the chitosan and MDOC layers adhered to one another. FIG. 14B provides a similar arrangement as that shown in FIG. 14A, except the adhesive material is delivered according to a sputter coating process. In both instances, the layers of materials will not be needed to be adhered to one another after the electrospinning process when being used in a wound dressing assembly. It has been further determined that, in particular, the use of a nanospun polyhydroxy butyrate melt or a similar low temperature melt (i.e. 50°-70° C.) provides sufficient adhesion for the individual layers of nanospun materials. Furthermore, it is possible to adjust the conditions and temperature that the material is melted at, thereby preserving the porosity between the individual layers of nanospun materials. Polycaproalctone is another possible adhesive material that can be used according to the present invention.

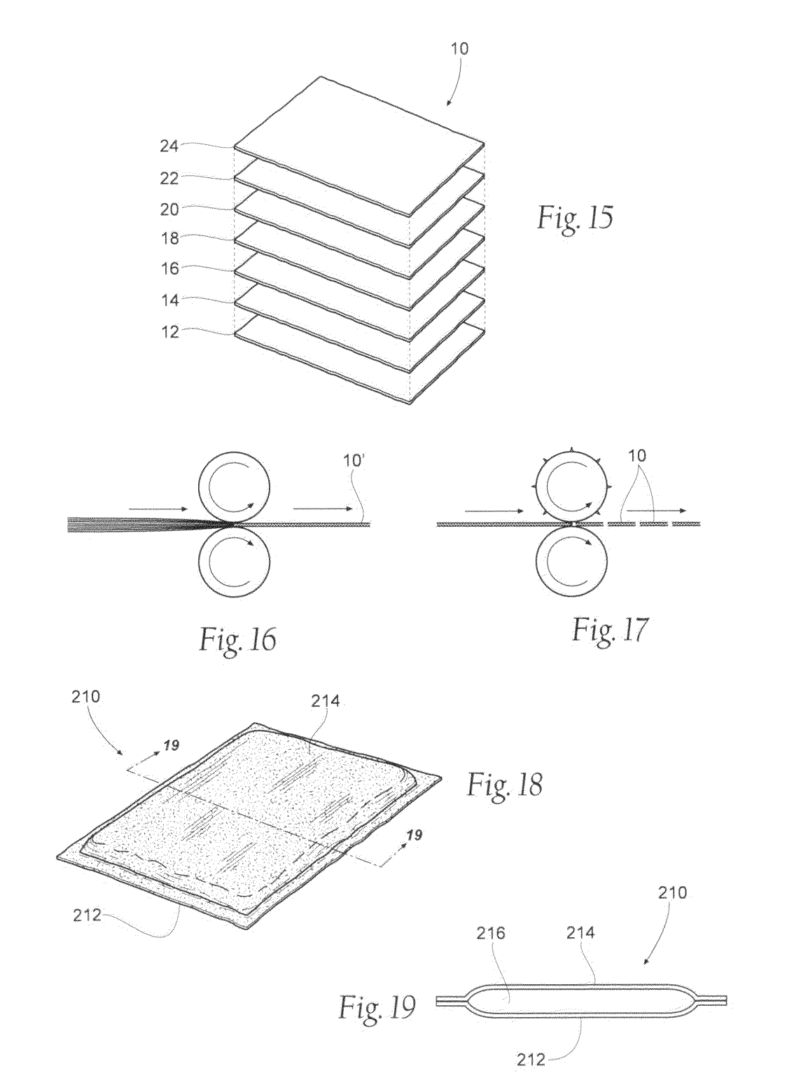

After the various nanofiber mats 130 have been formed, either as individual layers or as composite layers, the mats 130 will be used for the layers of the wound dressing assembly of the present invention. If necessary layers of adhesion may be used in between the layers of material. The layers will be positioned as required, as is shown in FIG. 15. Once the layers are in their desired ordered, the layers can be compressed together to form the wound dressing material 10′, as depicted in FIG. 16, which can then be further cut or severed, as depicted in FIG. 17, to form individual wound dressing assemblies 10. As an example, the compressed wound dressing material 10′ can comprise a sheet of material 300 cm×50 cm, which will then be cut down to individual size dressing assemblies 10 as desired. Other methods of combining the individual layers can also be used, such as using adhesive materials, as described in FIGS. 13-14B, or possibly using other forms of compression or mechanical adhesion to form the individual wound dressing assemblies. Combinations of the above can also be used, in forming one or more layers in the electrospun wound dressing assembly.

The wound dressing assembly 10 is one example of possible dressing assemblies developed according to the present invention. FIGS. 18 and 19 provide an alternate dressing assembly 210 according to the present invention. As stated above, dressing assemblies produced according to the present invention can be altered and varied, depending on the particular uses of the final dressing assembly. The therapeutic qualities of the dressing assemblies can be changed by using different electrospun materials or by providing additional layers of nanospun materials for the dressing assembly.

The dressing assembly 210 contemplates using different layers and, also, different materials. For example, the dressing assembly 210 comprises a first layer 212 consisting of a microdispersed oxidised cellulose material, commonly referred to as MDOC, with the MDOC material being formed by an electrospinning process. Likewise, the second layer 214, which is comprised of a chitosan material, is also formed through an electrospinning process. The interior layer 216 is comprised of a material that has therapeutic qualities, with the interior layer 216 in the form of various types of materials, such as a powder-based material or a nanomaterial that has been cut or chopped into smaller pieces as desired for a particular use. Alternatively, the interior layer 16 could also comprise could comprise another layer of nanospun material. As an example in the wound dressing assembly 10, the interior layer 216 is comprised of powder, which comprises a combination of MDOC and chitosan materials. The wound dressing assembly 210 may be assembled in a similar fashion as discussed with respect to the wound dressing assembly 10.

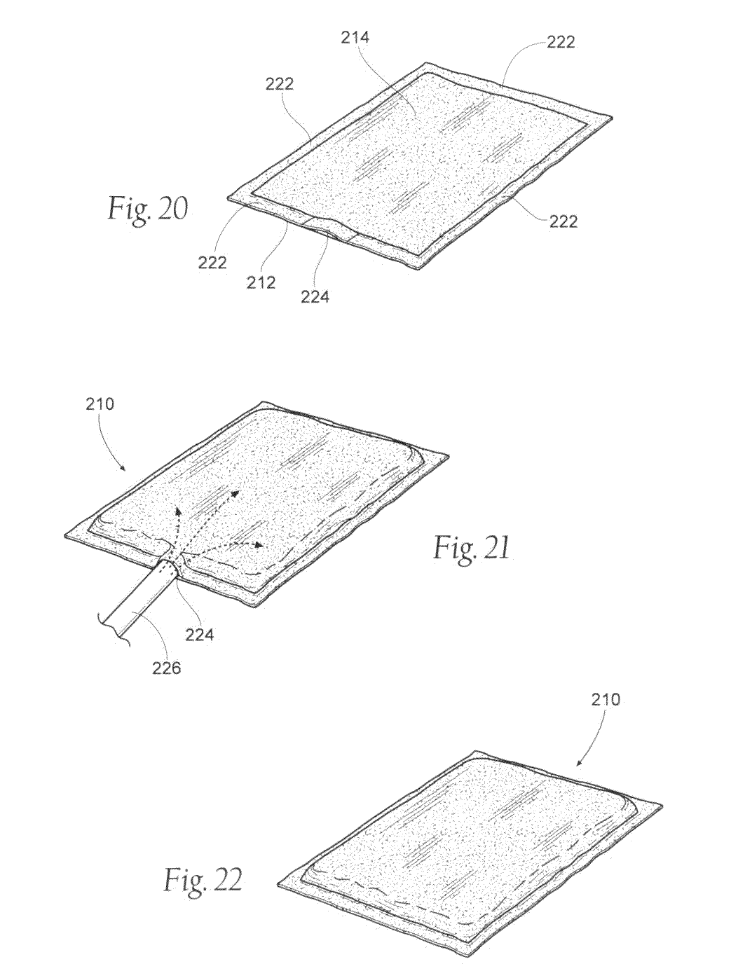

FIG. 20 depicts the layered dressing assembly 210, with the first layer 212 being joined to the second layer 214. The edges 222 of the layers 212 and 214 are joined together, with the central area of the layers 212 and 14 open. The layers 212 and 214 can be joined together with the use of adhesives, heat press processes or other welding processes, or possibly with the combination of an adhesive and a welding process. An opening 224 remains along the welded edges of the assembly 210, which allows the interior layer 216 to be inserted into the assembly 210.

FIG. 21 shows a tube 226 being inserted into the opening 242, which will allow the interior layer 16 to be injected into the layered dressing assembly 210. As noted, the interior layer could comprise a powder or a nanospun material that has been cut or severed into smaller pieces. Once a predetermined amount of material is inserted into the assembly 210 to form the interior layer, the tube 226 is removed, and the opening 224 is sealed, as shown in FIG. 22. As discussed with the sides 222 in FIG. 20, the opening 224 can be sealed with an adhesive, heat pressure or welding processes, or other sealing processes.

Alternatively, the layers 212 and 214 would pass through a machine that would allow the bottom layer 212 to be pulled downwardly to form a well for the material 216 to be placed, wherein the layer 214 would then be placed above the material 216 and sealed to the layer 212, as discussed. FIGS. 18-22 demonstrates that different arrangements of layered nanomaterials can be used to form a wound dressing assembly, and an assembly according to the present invention should not be limited by the order of the individual layers or the materials that make up the individual compounds or compositions that make up the individual layers. The present invention also could comprise an individual layer of electrospun material formed according to the present invention. The present invention provides an innovative process for tailoring a wound dressing assembly for specific needs, based on the various materials that potentially may comprise the wound dressing assembly. Section II describes methods and methodology for determining possible combinations of materials that can be used for producing and developing wound dressing assemblies for particular uses.

### II. PROCESS OVERVIEW FOR DETERMINING THE MATERIALS THAT COMPRISE THE LAYERED WOUND DRESSING

The present invention provides layered wound dressing assemblies that are formed from nanospun materials. The wound dressing assemblies are formed according to various prescribed conditions, such as whether the wound dressing assembly is to be hemostatic, antimicrobial, capable of delivering a drug, or a combination of various characteristics. Examples of characteristics that are considered in developing a wound dressing assembly also include regulating the moisture content of the assembly, controlling lateral wicking, and promoting vertical wicking through the assembly. Similarly, the wound dressing assembly may be required to be completely dissolvable or may be required to retain its shape while being used. For each instance, it may be necessary that the wound dressing assembly comprise different layers, as discussed above in section I. The present invention further contemplates methods and processes for determining possible materials to be used in a layered wound dressing assembly.

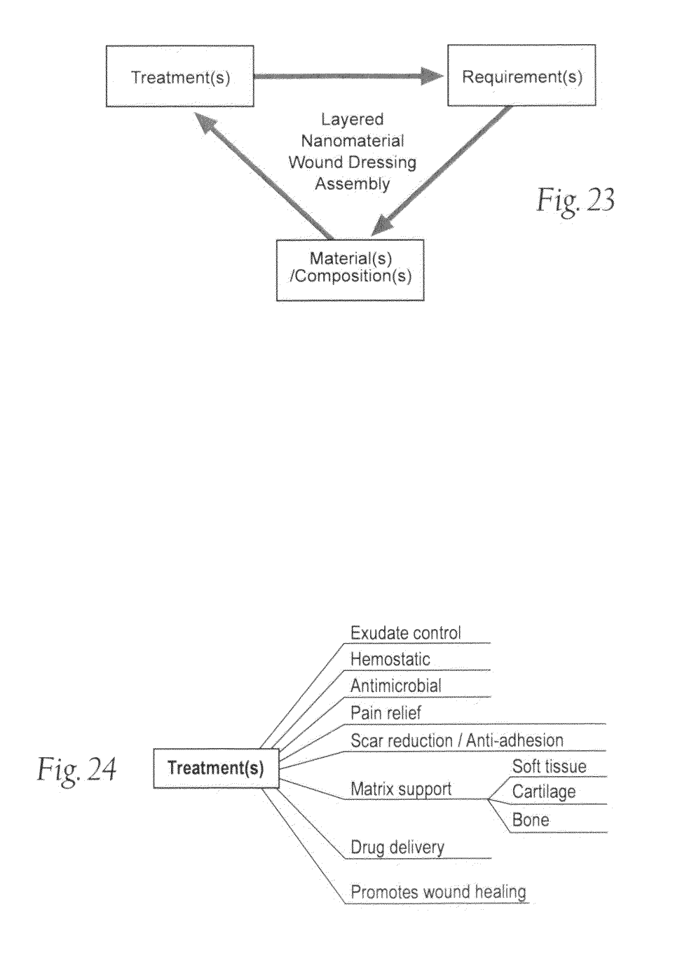

FIG. 23 provides an overview of the process for determining the materials required for a wound dressing assembly. FIGS. 24-26 provide further details to each of the categories shown in FIG. 23 for producing a wound dressing assembly. Each of the categories, Treatment, Requirement, and Material, provide gateways into the evaluating the requirements for the eventual final composition of the wound dressing assembly. Each of the three categories is also related to one another, to further assist in determining materials for a wound dressing assembly. As will be further evident from the discussion with respect to FIGS. 24-26, each of the three categories overlap one another. This will further assist the user in analyzing the proper materials for producing a specific wound dressing assembly. Because of the overlap, a specific material or composition will potentially appear in more than one of the three categories, which may allow the user to more quickly decide on the most efficient or useful material for a specific wound dressing assembly.

FIG. 24 discusses the wound dressing assembly parameters in the context of what the wound dressing assembly will be used for. That is, FIG. 24 discusses the potential treatments that may be needed for the wound dressing assembly. For example, the wound dressing assembly may be used for hemostatic purposes, pain relief, scar reduction, drug deliver, or more than one of these and other treatments. The treatment may be hemostatic or antimicrobial, in which case this may further provide options for the user to select a material. For example, if the dressing assembly is to provide antimicrobial features, the user may select any of various materials, such as a chitosan, chlorhexidine, or silver material, as depicted in FIG. 26. Further, as discussed below in section III, it may be possible to combine one or more of the materials into an individual layer of nanomaterial, which may provide hemostatic, antimicrobial, and/or other characteristics in the individual layer.

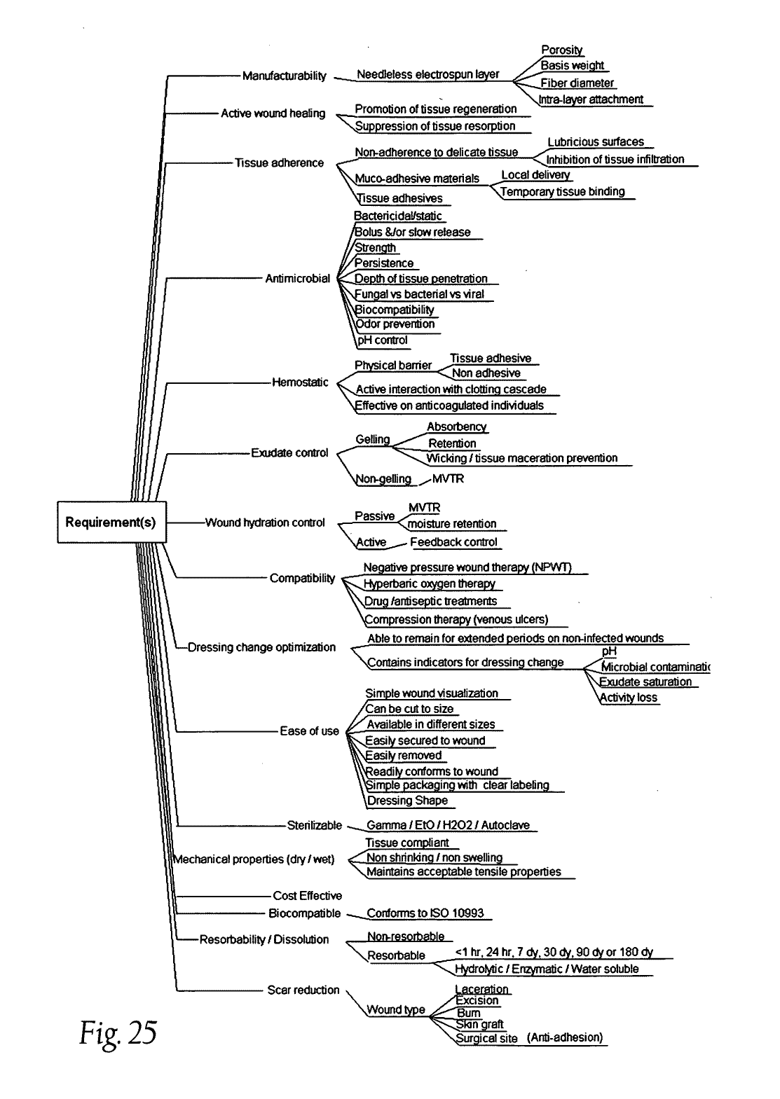

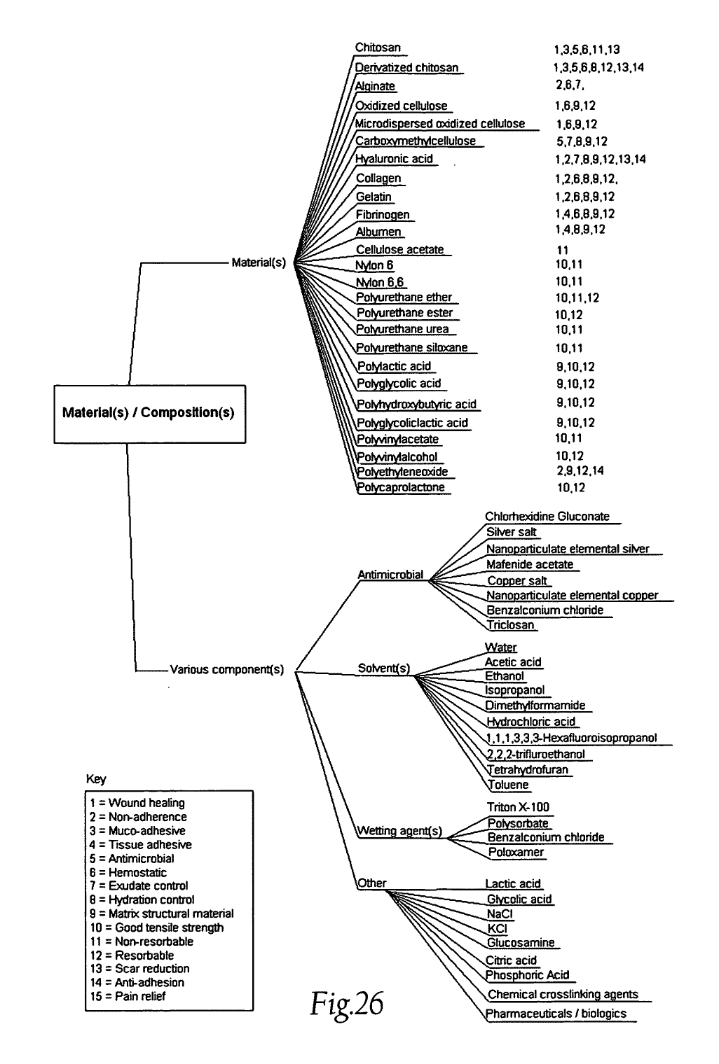

FIG. 25 provides guidance in determining the requirements for the specific layers that make up the wound dressing assembly and, also, provide guidance for the number of layers required in the wound dressing assembly. The assembly may incorporate a different material if the assembly is to be nonadhesive or adhesive. Likewise, the duration that dressing assembly should be in contact with the patient may help determine the material that is to be used. The wound dressing may require a material that will come into contact with blood and other biological fluids, or it may require a material that will dissolve quickly when coming in contact with the with fluids, or the requirements may require that the dressing assembly may be stable in an aqueous solution. Furthermore, the dressing assembly may require different layers, with each of these characteristics provided in a separate layer.

FIG. 25 also shows that the present system contemplates the makeup of the wound dressing assembly based on the physical requirements of the dressing assembly. In some dressing assemblies it is important that the structure is flexible, while in other structures the tensile strength may be an important feature. Based on the required criteria, the user can determine which particular materials would best be suited for a particular dressing assembly. Likewise, the parameters depicted in FIG. 25 help the user ascertain an efficient arrangement of a wound dressing assembly, by providing tools for a person to cross-reference the requirements for the wound dressing, such as materials, physical characteristics, and the structure of individual layers.

FIG. 26 provides a flow chart further depicting the possible materials that may comprise a wound dressing assembly. When compared to the portions of the processes shown in FIGS. 24 and 25, it can be seen that there is some overlap between the various Figures, particularly overlap with the selected materials. As noted in the key in FIG. 26, each of the various listed materials has various qualities that address the treatments and requirements that can be found in either or both of FIG. 24 and/or FIG. 25. This allows for further fine tuning of the desired layers for a wound dressing. For example, after the various characteristics and the various layers are assessed in FIGS. 24 and 25, FIG. 26 allows for refinement of the characteristics for each of the selected materials. For example, it may be determined that a wound dressing assembly may be needed that has resorbable layers that are hemostatic and also potentially a non-adhering layer that has exudates control. As such, a layer of chitosan material, with a layer of polyethylene oxide (PEO) material, along with a layer of hyaluronic acid may be selected. Once a specific material is determined for a layer, i.e. chitosan crosslinked to polyethylene oxide (PEO), the material characteristics, such as the particular basis weight, can be determined, so that the parameters are set for determining the electrospinning process for that material, as will be discussed further with respect to the material qualities, as discussed in section III. Further, as noted by the relation in FIG. 23, the individual layers can be further refined by working through the process delineated in FIGS. 24-26 to further provide an efficient dressing assembly that can provide multiple therapeutic qualities.

It should be understood that the processes depicted in FIGS. 23-26 are exemplary of materials and qualities that could be incorporated into wound dressing assemblies. Other properties for particular materials could be incorporated into the selection method for a wound dressing and for determining the electrospinning parameters of the process. It should also be understood that the disclosed process is an overview for which further materials can be incorporated into the outline. The method provides a structured process for determining the materials and characteristics used for a wound dressing assembly, which can result in a more efficient production process than prior art methods.

The following section provides further details with respect to some of the various materials noted above for use for individual layers of a wound dressing assembly, including potential uses for the materials.

### III. VARIOUS MATERIAL CHARACTERISTICS

Wound dressing assemblies according to the present invention can be made from a variety of materials that have various therapeutic, hemostatic, or other efficacious and helpful qualities. To provide various therapeutic regimens, different layers of electrospun materials are provided, with different layers comprising different materials. While the individual layers have been directed at electrospun materials that generally consist of a single material, such as chitosan, MDOC, collagen, carboxymethyl cellulose (CMC), gelatin, hyaluronic acid, etc., the individual spun layers may comprises a composition of more than one compound or material. Various examples of materials that can be electrospun and used according to the present invention include: chitosan compounds, alginate compounds, cellulose compounds, including oxidized cellulose compounds, mircodispersed oxidized cellulose compounds, carboxymethyl cellulose compounds, hydroxyethlyl, cellulose compounds, methyle cellulose compounds, and ethyle cellulose compounds collagen compounds, fibrinogen compounds, albumen compounds, cellulose acetate compounds, nylon compounds, polyurethane compounds, polyurethane ester, ether, urea, and siloxane compounds, polyactic acid compounds, polyglycolic acid compounds, polyhydroxybutyric acid compounds, polyglycoliclactic acid compounds, polyvinyl alcohol and alcohol compounds, polyethylene oxide compounds, gelatin compounds, hyaluronic acid compounds, polycaprolactone compounds, silk compounds, pectin compounds, polyacrylic acid compounds, starch compounds, and combinations thereof.

The following examples discuss various examples of electrospun materials that comprise more than one material or compound, including compounds that may be cross-linked with other compounds.

A. General Examples of Electrospun Materials

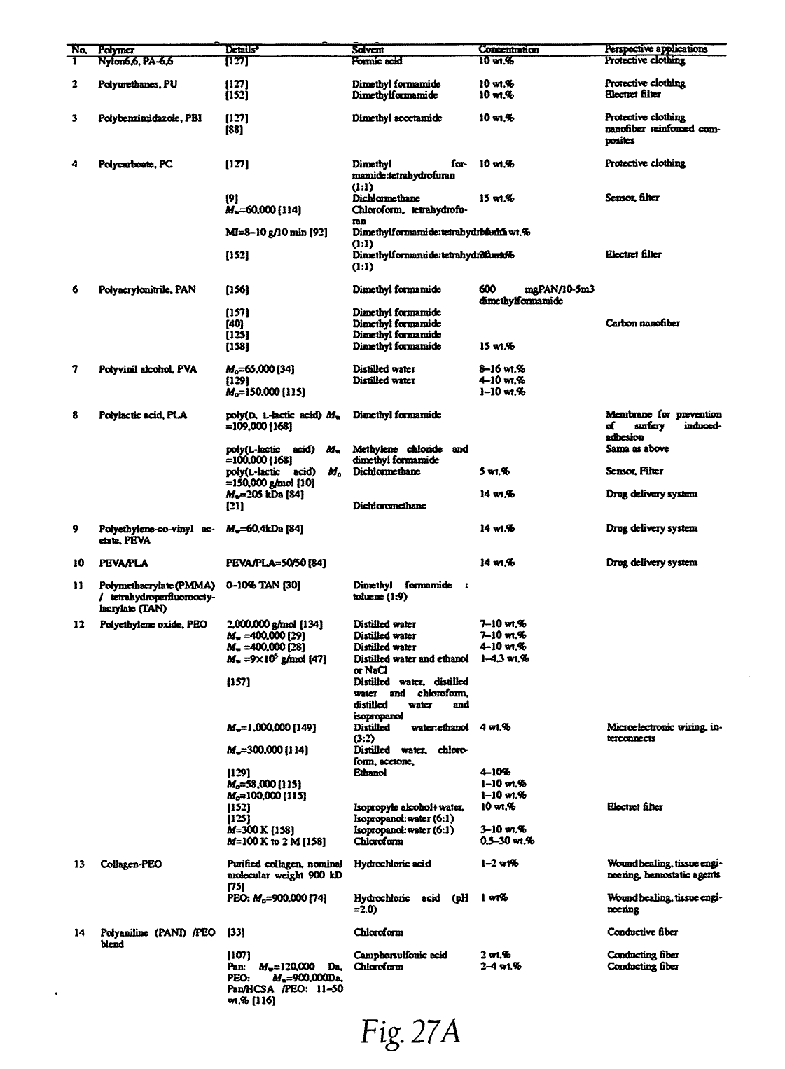

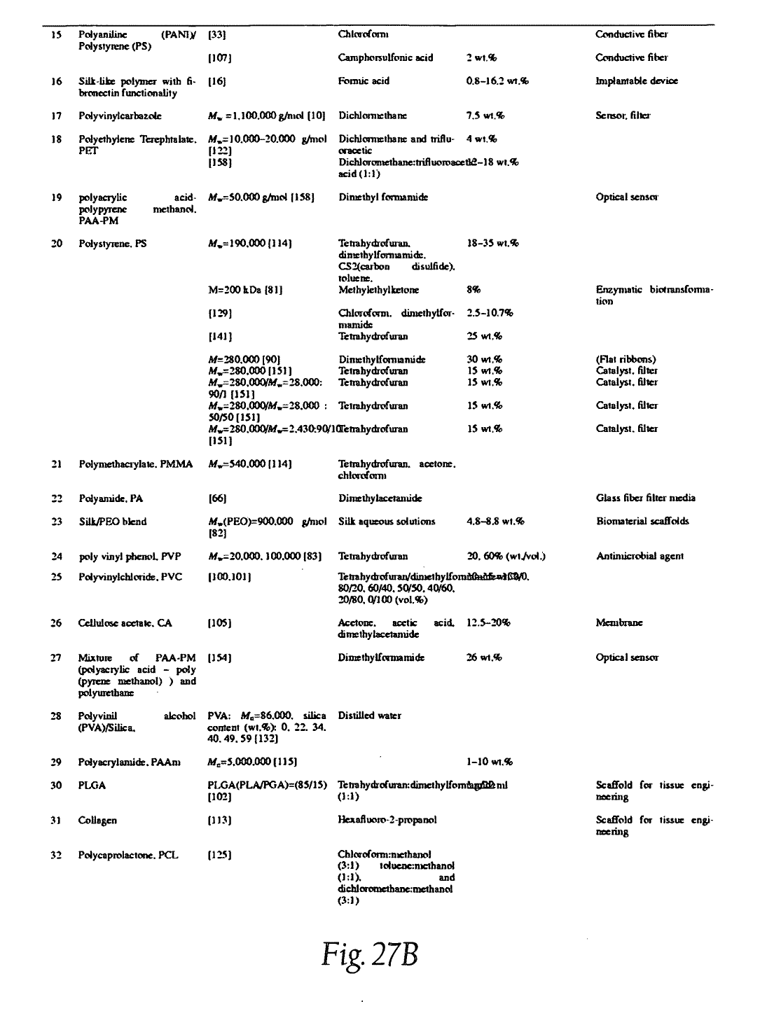

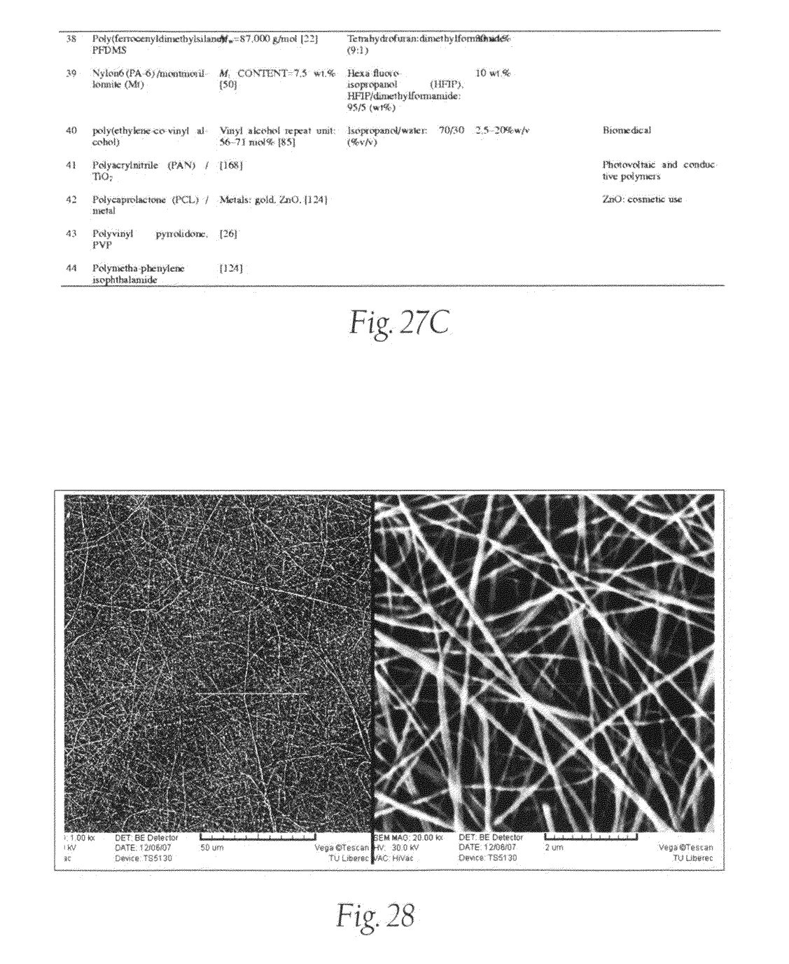

Various materials are shown and described in the table shown in FIGS. 27A-27C, which refer to potential materials that have been known to be able to be electrospun. Each of the materials has been used to form nanospun fibers and layers of nanomaterials. As noted in the table, the materials have different properties and characteristics, which could be combined to provide a wound dressing assembly having more than one therapeutic quality. For example, if it was desired to provide a wound dressing assembly that was capable of drug delivery while promoting wound healing, a polyethylene co-vinyl material (PEVA) may be selected as one layer of material, while a second layer of material may comprise a collagen/polyethylene oxide (PEO) composition. The listed materials are exemplary of possible materials and combinations that could be used. However, other materials and nanomaterials can be spun. The following section discusses another example, an electrospun material comprising chitosan and PEO.

B. Electrospun Chitosan/Polyethylene Oxide (PEO) Matrix

1. Matrix Overview

Chitosan and polyethylene oxide were combined to form a nanofiber matrix. The chitosan material was prepared from solution. As an example, the chitosan selected for processing into the matrix has a viscosity at 25° C. in a 1% solution of acetic acid (AA) with spindle LVI at 30 rpm, which is about 100 centipoise to about 2000 centipoise. More preferably, the chitosan has viscosity at 25° C. in a 1% solution of acetic acid (AA) with spindle LVI at 30 rpm, which is about 125 centipoise to about 1000 centipoise. Most preferably, the chitosan has viscosity at 25° C. in a 1% solution of acetic acid (AA) with spindle LV1 at 30 rpm, which is about 150 centipoise to about 500 centipoise. The chitosan solution could then be combined with the PEO in solution prior to the electrospinning process.

The following results are directed towards an electrospun material having a Chitosan/Polyethylene oxide (PEO) product in the final electrospun fiber with a weight ratio of 90% to 10% respectively. The resultant product was tested and determined to be soluble with a mean fiber diameter of 137±31 nm. An example of a scanning electron microscope (SEM) picture of a Chitosan/Polyethylene (90/10) oxide product at 2 different magnifications (1000× and 20,000×) is shown in FIG. 28. The tensile strength of the samples was measured and any effect of gamma irradiation examined on the tensile strength was also recorded.

Since the eventual electrospun nanomaterials will be used in wound dressing assemblies and other wound care products and compositions, it is important that sterilization of the product did not have a detrimental effect on the integrity of the sample. That is, it is preferable that sterilization of the materials does not affect the physical parameters and dimensions of the material. Table 1, below, compares a chitosan/PEO nanomaterial matrix that has been sterilized with gamma radiation to a chitosan/PEO nanomaterial matrix that has not been sterilized. Sterilization is generally comprises a gamma radiation between 11-40 kGy, with a preferred gamma radiation being 25 kGy. The tensile strengths of the matrixes were compared. As the results indicate, no detrimental effects occurred from the sterilization process.

TABLE 1
Effect of gamma irradiation on the tensile
strength of Chitosan nanofibers

| | | | | | Strength | Elongation |
|---|---|---|---|---|---|---|
| | | Average | | Average | related to | related to |
| | | basis | Average | elon- | the basis | the basis |
| | Sterilized | weight | strength | gation | weight | weight |
| Sample | (Y/N) | [g/m²] | [N] | [%] | [N/g/m²]] | [%/g/m²]] |
| Chitosan | N | 15.67 | 16.49 | 4.88 | 1.05 | 0.31 |
| 90/PEO | Y | 16.22 | 17.46 | 4.85 | 1.08 | 0.30 |
| 10 | | | | | | |

The chitosan/PEO matrix was also measured at various fiber diameters, to determine if the physical characteristics would be altered, which potentially could affect the therapeutic qualities of the matrix.

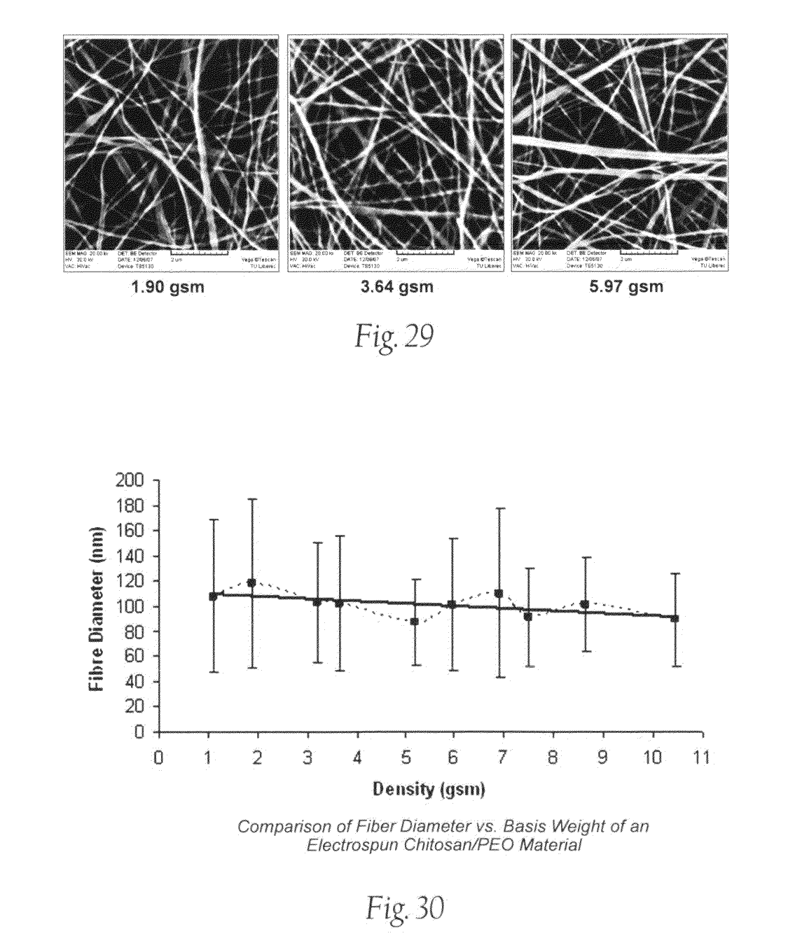

Preferably, the fiber diameter will be consistent, regardless of the basis weight of the fibers, which will provide for more consistent qualities for the chitosan/PEO matrix and analysis of the chitosan/PEO matrix, since the therapeutic qualities will not change significantly if the fiber diameter is altered. FIG. 29 shows SEM images of chitosan/PEO nanofiber matrices having various densities, and FIG. 30 graphically compares the fiber diameters at various densities.

It can be seen in FIG. 30 that there is no change in the fiber diameter with increasing product basis weight. This is an important property of the process as it means that any surface area related phenomenon will be retained even if the nanofibers meshes are produced at different basis weights of product.

To form the chitosan/PEO matrix, the chitosan and PEO of the invention should be combined in a suitable fashion. Section 2 below provides possible suitable ways for the compounds to be cross-linked with one another.

2. Cross-Linking of the Chitosan and Poly-Ethylene Oxide

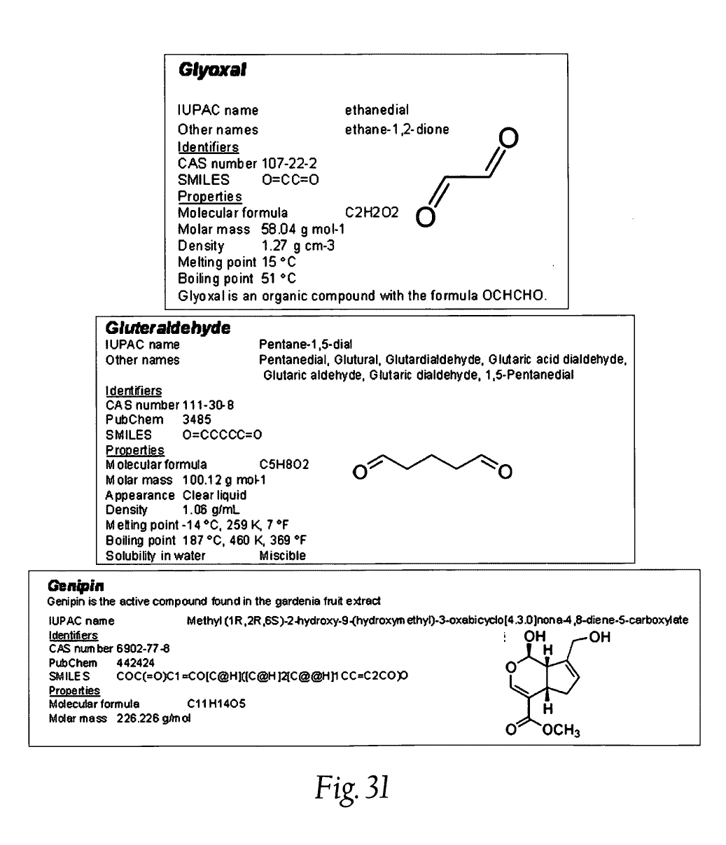

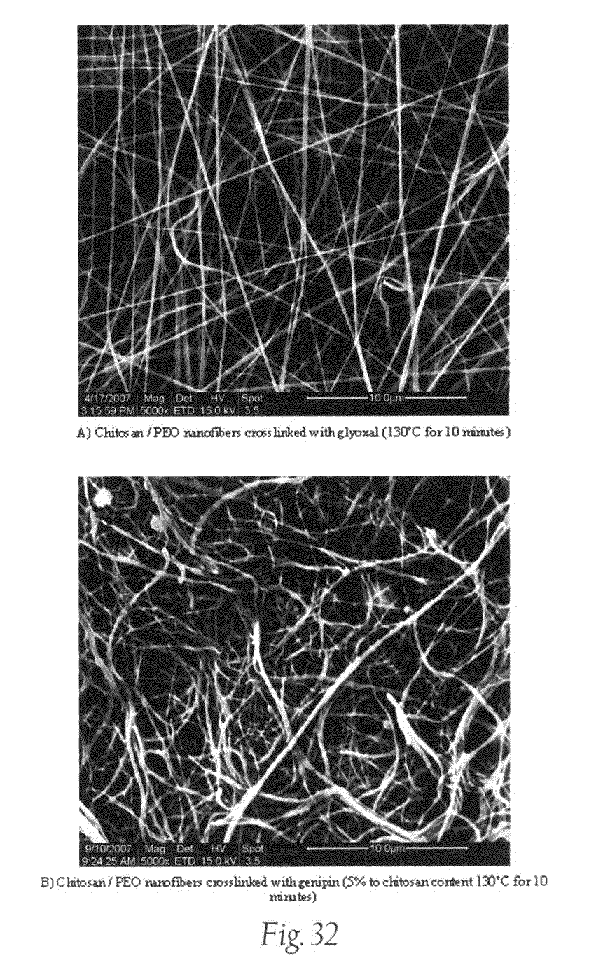

As stated above the nanofiber chitosan product produced by the present invention is soluble in water. This largely renders the product ineffective for use as a hemostat due to the almost immediate dissolution of the product when in contact with blood. One way to convert the soluble nanofiber product to an insoluble product is to crosslink it. Crosslinking is the formation of chemical links between molecular chains to form a three-dimensional network of connected molecules. A substance that promotes or regulates the intramolecular covalent bonding between polymer chains, linking them together to create a connected structure, is called a crosslinking agent. A number of crosslinking agents were used in this project to crosslink the chitosan/PEO nanofiber product. These included glyoxal, gluteraldehyde and genipin. Details of these crosslinking agents can be found in FIG. 31.

The most common crosslinking agents, or crosslinkers, used with chitosan are dialdehydes such as glyoxal and glutaraldehyde. These crosslinking agents were used successfully at 130° C. for 10 minutes. The resulting covalently crosslinked fibers were rendered insoluble and were suitable for use as in stabilizing electrospun materials for use in wound dress applications. It should be understood that care must be taken when using these cross-linking compounds, since they are considered toxic above certain concentrations. For example, glutaraldehyde is known to be neurotoxic, and its fate in the human body is not fully understood. Also, glyoxal is known to be mutagenic. Therefore, use of such crosslinking agents requires purification before administration, and potential testing of the presence of free unreacted dialdehydes in the products prior to administration. It should be noted that materials and compositions are routinely heated to elevated temperatures (i.e. 130° C.), with such materials also generally not breaking down due to dissolution. As such, it should be understood that there is the potential that other factors may affect the relationship of the materials, with similar appearances as the crosslinked compounds.

Crosslinkers such as diethyl squarate, oxalic acid or genipin can exhibit direct crosslinking mechanisms, although they remain incompletely elucidated.

The use of genipin is an alternative to dialdehydes as it is a naturally occurring material, which is commonly used in herbal medicine and as a food dye.

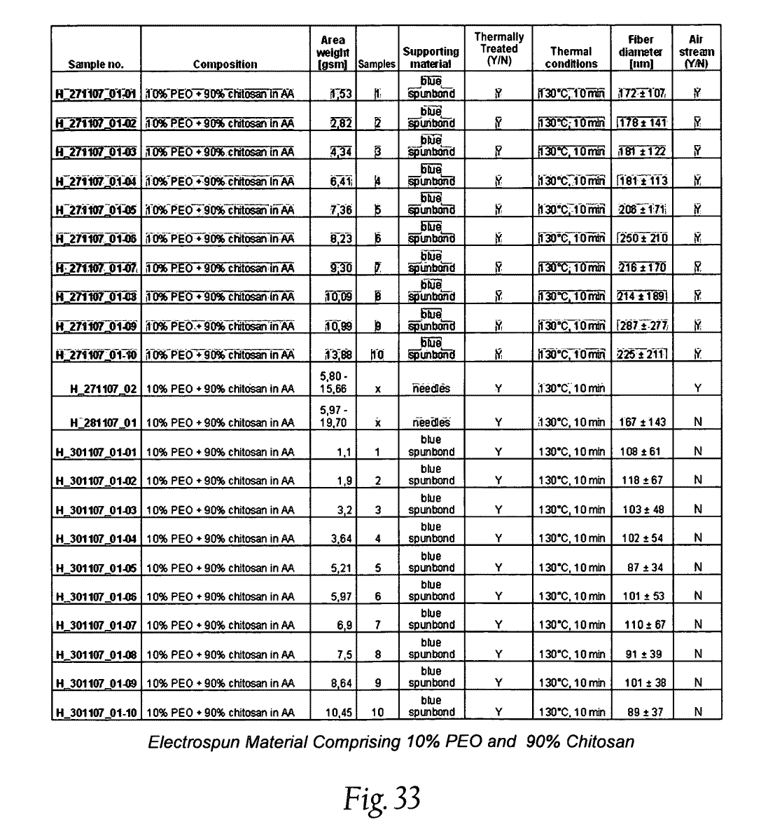

Because of potential toxicity limitations of the above described covalent crosslinking approach, other crosslinking processes have also been considered to tend the nanospun materials insoluble. One such process is thermal treatment of the nanospun construct after electrospinning. Thermal treatments of the chitosan/PEO samples were carried out by oven heating the nanofibers at a range of temperatures for 10 minutes. Temperatures from 70° C.-200° C. were examined and the solubility of the samples subsequently analyzed. From this analysis it was concluded that post nanofiber production treatment at 130° C. for 10 minutes gave the optimum sample. SEM images of examples of such products can be seen in FIG. 32. Various samples are shown and compared in FIG. 33.

It should be understood that the chitosan/PEO matrix can be formed with or without actual thermal treatment of the chitosan and PEO materials, as well as the fact that matrices at elevated temperatures may not necessarily be rendered insoluble. Depending on the particular uses and the desired requirements of a particular matrix or composition, crosslinked or non-crosslinked, thermally or non-thermally treated, nanospun constructs may be advantageous. The following example discussed thermally treated electrospun chitosan/PEO constructs.

### Example

### Electrospinning of Chitosan and Polyethylene Oxide (PEO)

The following example examines chitosan material that has been mixed in a solution with polyethylene oxide (PEO) with subsequent investigation of the effect of thermal-treatment on chitosan/PEO matrices.

The electrospun solution was comprised of a mixed combination of a chitosan solution and a PEO solution. The chitosan solution comprised 7.5 g of chitosan material (Chitosan AK) which was mixed with 60.1 g of deionized water and subsequently mixed with 100 mL of acetic acid and mixed with a magnetic stirrer to form an aqueous solution. The PEO solution was formed by combining 0.7 g of PEO (MW 400,000, purchased from Scientific Polymer Products, Inc., Ontario, Canada) with 24.3 g of deionized water. The chitosan solution was added to the PEO solution to form the solution to be electrospun.

The electrospinning solution was also distributed over polyethylene (PE) foil and allowed to dry. The foils were to be further used in infrared (IR) spectroscopy analysis to further study structural and chemical changes in the chitosan/PEO matrix.

The following parameters were used for the electrospinning process:

### Equipment: NanoSpider LAB, Tisnov, Czech Republic

Electrode: Small smooth cylinder
Collector: Needles+Fixed spunbond

### Electrode Distance: 130 mm

Fabric speed: 0 m/min.
Electrode spin: 3.2 min⁻¹

### HV: 82 kV

The chitosan/PEO solution was spun for approximately one (1) hour, with the obtained materials being orientated in a generally two-dimensional, planar fashion. The spun material was divided into four groups, with each of the groups further treated, as follows:

a) no further treatment;

b) heated to 105° C. for 1 hour;

c) heated to 105° C. for 1 hour, followed by heating to 150° C. for another hour; and

d) heated to 150° C. for 1 hour.

The chitosan powder used for forming the solution and the chitosan foils were treated in the same fashion.

### Results

Loss on Drying: Approximately 20% of the volatile compounds (i.e. acetic acid, water) were lost when the materials were dried at 105°, but less than 1% of the volatile compounds further evaporated from the material when the material was further dried to 150° C., indicating that heating to 105° C. is sufficient to remove substantially the volatile compounds. Since acetic acid forms a substantially non-volatile acid salt with chitosan in the absence of water, small amounts of non volatile acetic acid may reside in the chitosan.

Dissolution: The chitosan powder and electrospun samples were tested for dissolution in water and 3% (w/w) acetic acid. All the thermally-treated samples were insoluble in water, while the non thermally-treated samples swelled when placed in water. All of the samples dissolved in the acetic acid, with each of the samples treated to 105° C. dissolving in approximately 5 minutes and the samples heated to 150° C. dissolving in approximately 2 hours. There were no significant differences (in terms of final water solubility) between the samples heated directly to 150° C. and the samples heated to 105° C. and then to 150° C. The significantly slower rate of dissolution in acetic acid of the 150° C. heat treated samples compared to the 105° C. treated samples may be associated with annealing of chitosan structure to a more paracrystalline isoform (possibly with greater hydrogen bonding) which is more resistant to dissolution. Treatment of chitosan acid salts in methanol produces similar annealing effects and dissolution resistance. The fact that the 150° C. sample did eventually dissolve in the acetic acid solution indicated that the thermal treatment did not induce covalent crosslinking in the chitosan

Infrared Spectroscopy:—Infrared spectroscopic analyses of the foils did not reveal any substantial differences between the heat-treated and the non heat treated samples.

Thus, as noted above, the example demonstrates that there is no covalent crosslinking associated with thermal treatment of chitosan/PEO but rather there is volatilization of acid salt and likely annealing of paracrystallinity in the polymer.

The following section contemplates and compares the potential of both thermally treated and untreated chitosan/PEO matrices, with respect to the antimicrobial activity of each. Further, application of thermal treatment is contemplated to affect the antimicrobial activities of the compositions.

3. Enhancement of the Chitosan/PEO Matrix For Increased Antimicrobial Activity

The antimicrobial activity of chitosan and its derivatives against different groups of microorganisms, such as bacteria and fungi, has received considerable attention in recent years. A large number of publications have demonstrated the antimicrobial activity of chitosan. The development of an antimicrobial chitosan wound dressing would be advantageous due to the elimination of the complications in wound healing associated with infection.

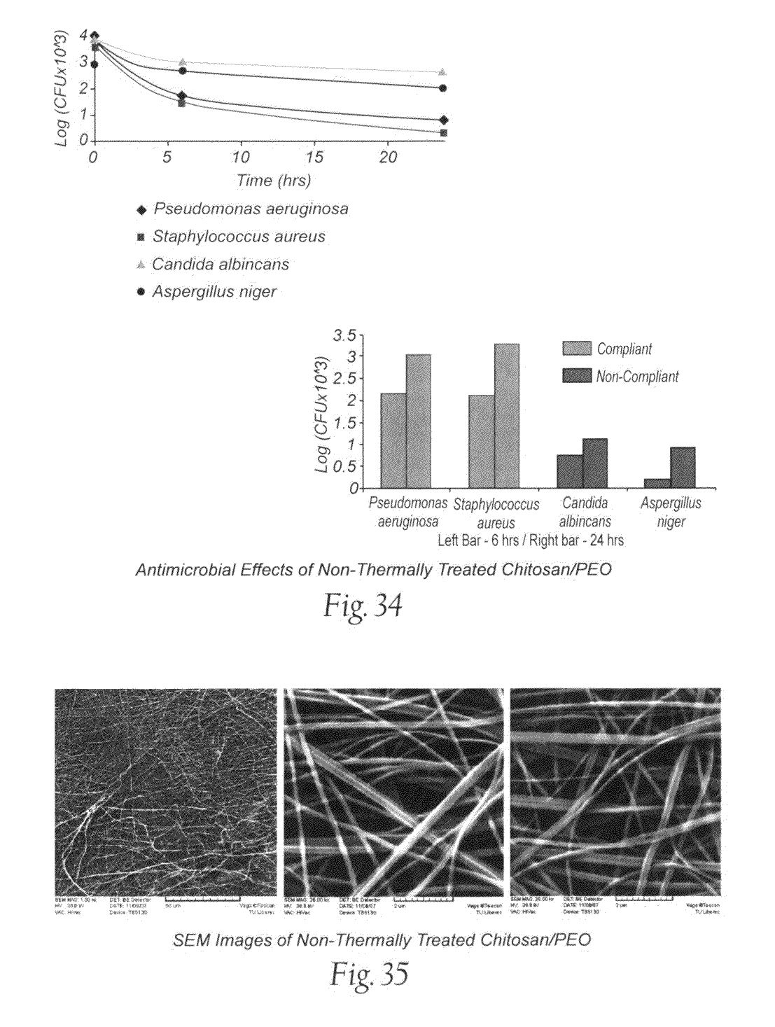

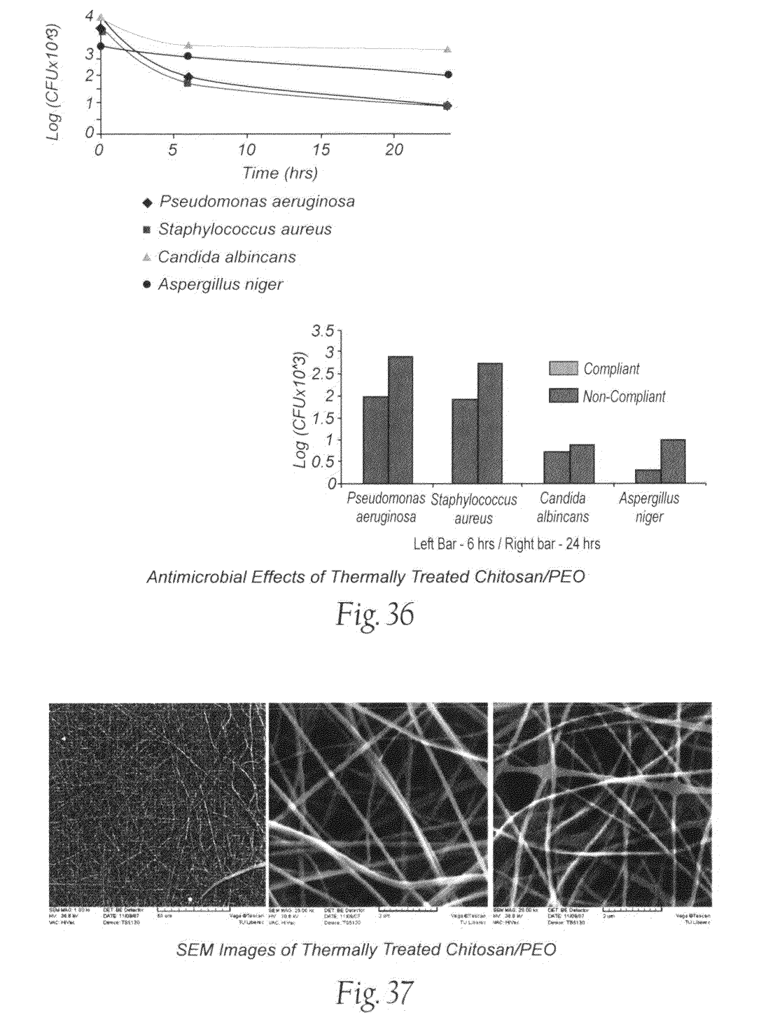

The nanofiber Chitosan/PEO matrix discussed above was examined for its antimicrobial activity (see FIGS. 34-37). Both thermally-treated and non thermally-treated matrices were tested. The Chitosan/PEO product was tested for antimicrobial activity against the topical microorganism strains Pseudomonas aeruginosa CCM 1961 (bacteria), Staphylococcus aureus CCM 4516 (bacteria), Candida albicans CCM 8215 (fungi) and Aspergillus niger CCM 8222 (fungi). The analysis method used was the US Pharmacopeia 28 for measurement of antimicrobial activity (antibacterial and antifungal). Table 1A shows compliance limits for the testing.

TABLE 1A
Compliance Testing Limits.

| | Decrease of cfu number (log) | |
|---|---|---|
| Finding/ | Hours 6 | Hours 24 |

| limit | Finding | Limit | Finding | Limit |
|---|---|---|---|---|
| Bacteria | x | >2 | x | >3 |
| Fungi | x | >1 | x | >2 |

FIG. 34 shows the antimicrobial effects of an electrospun non thermally treated chitosan/PEO material against the noted microbes. The effects were measured after 6 hours and after 24 hours. As shown, the material was compliant against Pseudomonas aeruginosa CCM 1961 (bacteria) and Staphylococcus aureus CCM 4516 (bacteria), but not compliant against Candida albicans CCM 8215 (fungi) and Aspergillus niger CCM 8222 (fungi). In comparison, the electrospun thermally-treated chitosan/PEO material, which is depicted in the graph in FIG. 35, was non-compliant for each of the four tested microbes.

As can be seen from the results neither non thermally treated nor thermally treated Chitosan/PEO matrixes met with the definition of antimicrobial as defined by the USP28. Although neither had the required antifungal activity it can be seen that the non thermally treated sample was indeed antibacterial. To enhance the antimicrobial qualities of the matrices, two systems were developed. The first system incorporates silver into the Chitosan/PEO product. This initially involved the dispersion of nano silver particles into the PEO component prior to an electrospinning process, such as the electrospinning process discussed previously. After the electrospinning process is performed, a thermal treatment step was further used on the materials.

Silver has been recognized for centuries for its antimicrobial properties (Russell and Hugo, 1994; Klasen, 2000; Lansdown, 2002). References to the use of silver for the treatment of chronic wounds date back to studies on ulcers in the 17th and 18th centuries (Klasen, 2000). Silver acts as a heavy metal by impairing the bacterial electron transfer system and some of its DNA functions (Russell and Hugo, 1994; Cervantes and Silver, 1996). In order to do this the silver ions must be able to enter the cell as the correct concentration in solution (Demling and DeSanti, 2001).

The second system employed to develop an antimicrobial version of the Chitosan/PEO matrix was to use different acids other than acetic acid in the electrospinning process. When subsequent thermal treatment was performed, it was anticipated that these acids would result in the formation of a polycationic structure that would have antimicrobial activity since lactic acid is significantly less volatile than acetic acid.

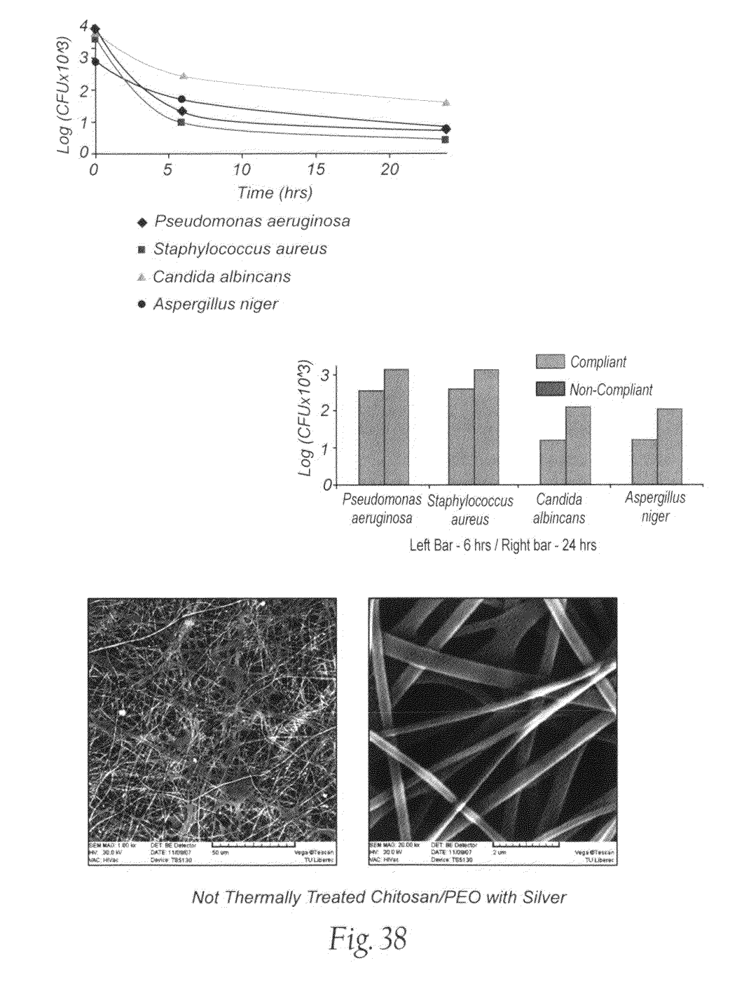

In comparison, the addition of silver to the chitosan/PEO matrices, both the non thermally treated matrix (FIG. 38) and the thermally treated matrix

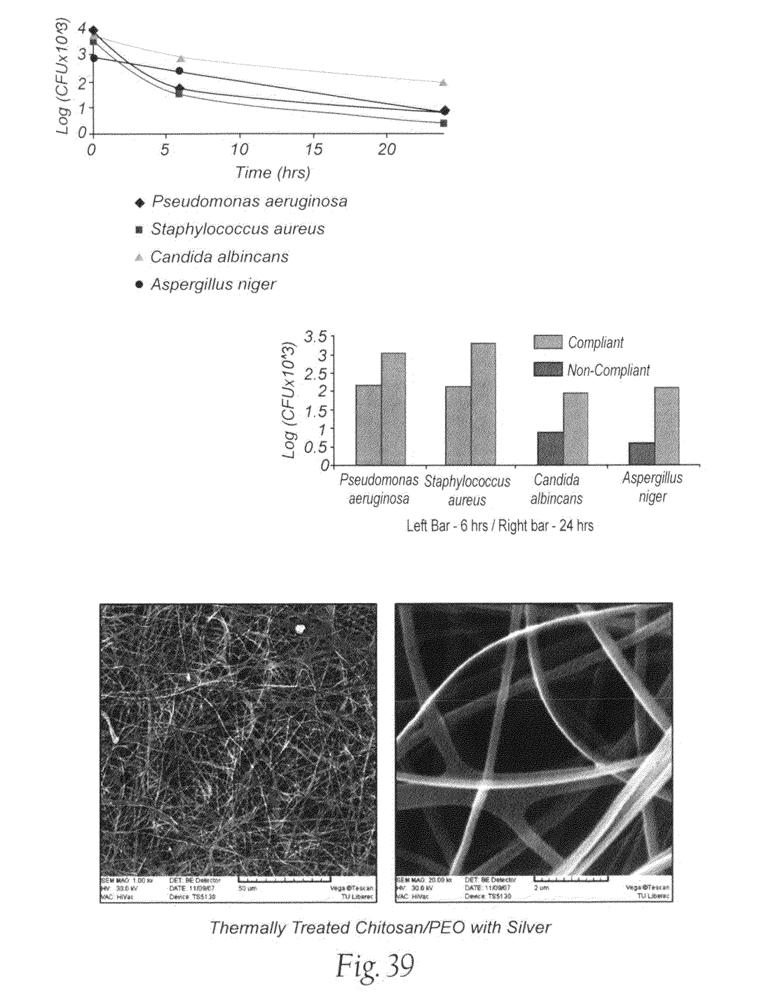

(FIG. 39), increased the compliance for the materials against all four of the tested microbes. However, the thermally treated material, represented in FIG. 39, was not completely compliant against Candida albicans and Aspergillus niger CCM 8222 (fungi) over the entire time range.

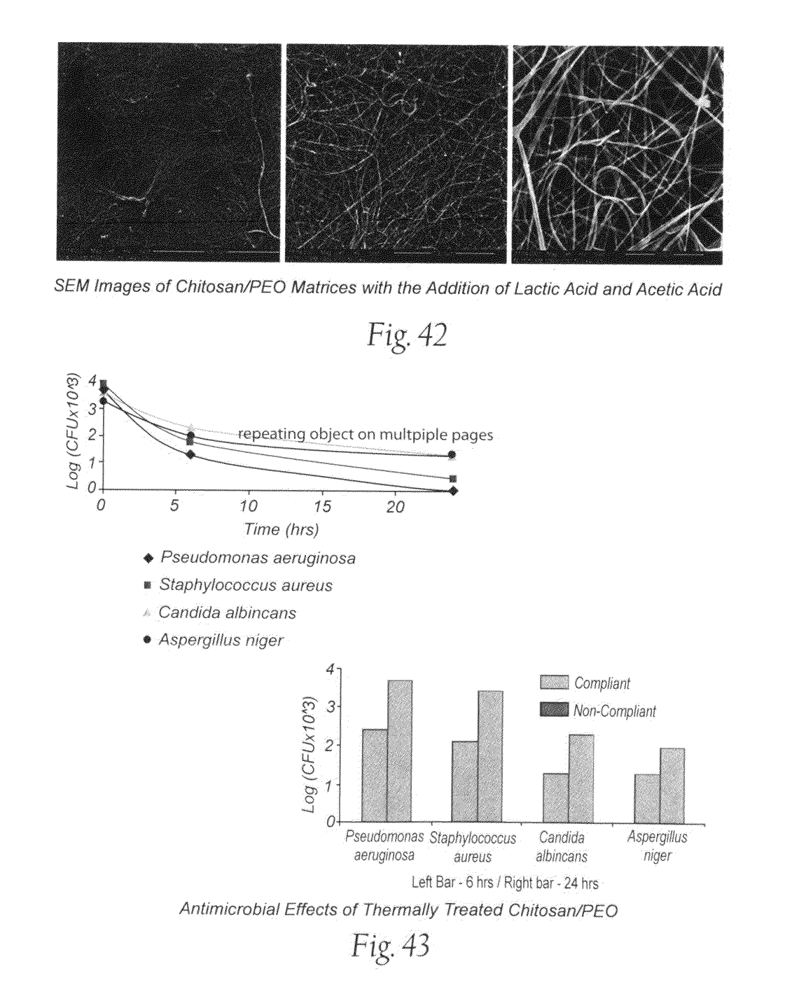

In FIG. 43, the presence of lactic acid and acetic acid in the solution used to prepare the thermally treated chitosan/PEO matrix indicates that the material were compliant against all four microbes over the entire time range.

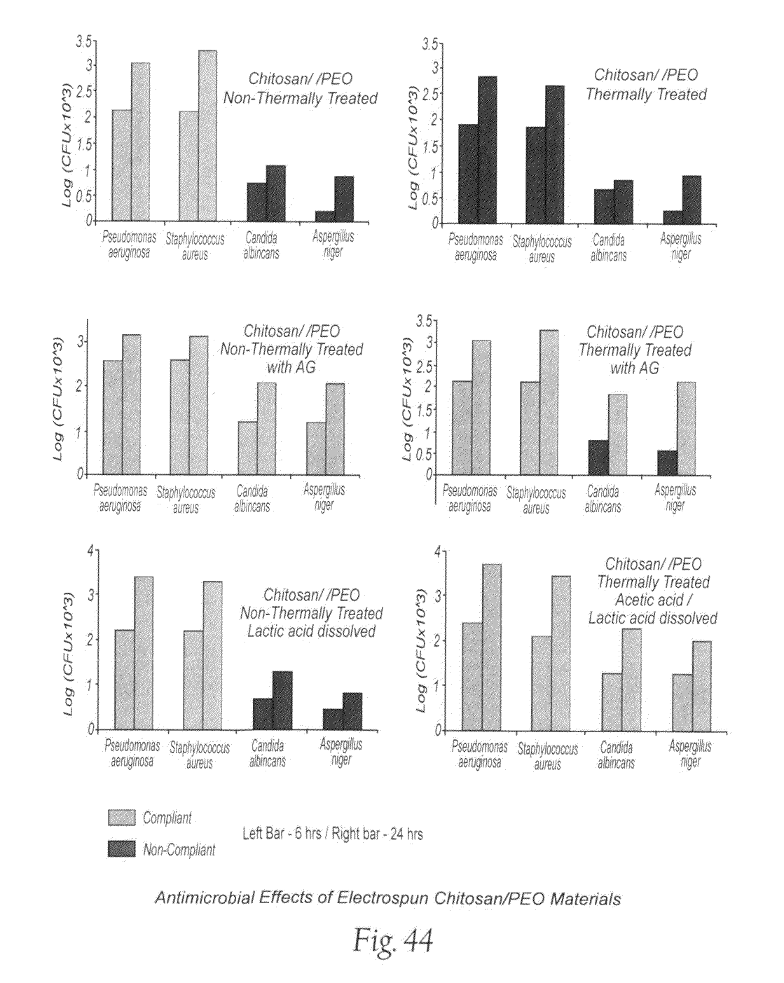

All of the tested matrices are shown and compared in FIG. 44. As is shown, the initial results indicate that the addition of the stated additives, silver, acetic acid, and/or lactic acid, improve the efficacy of the electrospun chitosan/PEO materials, both with the thermal treatment and the without thermal treatment.

Both the systems, silver and acetic acid/lactic acid are described below in further detail.

a. Addition of Silver to the Chitosan/PEO Matrix

Prior to the electrospinning process, approximately 20 ppm of SmartSilver® silver nanoparticles, provided by NanoHorizons, Inc., located in State College, Pa., was added to the chitosan/polyethylene oxide compound. The antimicrobial activity was measured as before and the results of a representative non-crosslinked products are displayed in FIG. 38.

The antimicrobial tests performed demonstrated that the product was indeed both antibacterial and antifungal. It met with the USP 28 definition of an antimicrobial composition. Previously it was seen that the non-thermally treated Chitosan/PEO matrix without silver was antibacterial but not antifungal. The addition of silver to the matrix conferred antifungal activity and ability onto the matrix.

The silver containing Chitosan/PEO product was also thermally treated, as previously described and the resulting product was tested for antimicrobial activity.

As demonstrated in FIG. 39 the thermally treated silver containing Chitosan/PEO product was not antimicrobial. However, it was also shown that the thermally treated sample was antibacterial. Similarly to the thermally treated sample without silver, the product was shown not to be antifungal.

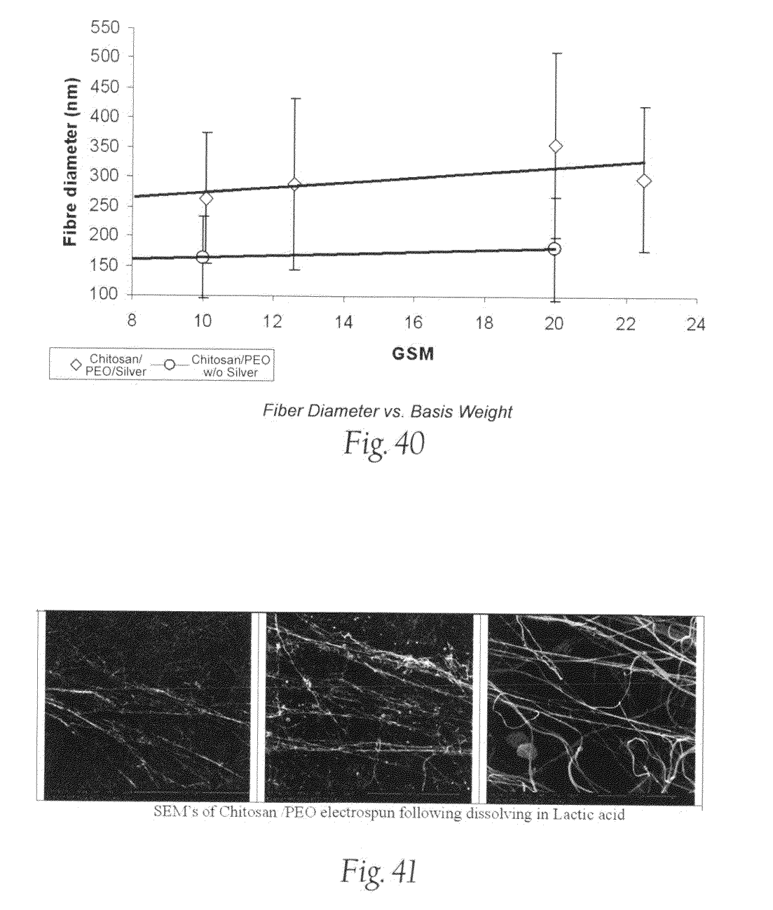

Furthermore, it was also evident from examination of the electrospun chitosan nanofibers that the fibers produced in the presence of silver were of a higher fiber diameter (see Table 2). Therefore a simple investigation was performed with standard electrospun chitosan nanofibers and electrospun silver containing nanofibers under the same conditions at a number of different basis weight. The results can be seen in FIG. 40.

TABLE 2
Fiber diameters of standard and silver
containing nano dressings

| | Standard | Silver containing |
|---|---|---|
| Non-Crosslinked | 164 ± 69 | 356 ± 156 |
| Crosslinked | 165 ± 99 | 305 ± 145 |

The results indicate that the fiber diameter of the electrospun chitosan increases by approximately 100 nm in the presence of silver. With increasing basis weight the diameter of the fibers also increases.

b. Use of Alternative Acids in Producing the Chitosan/PEO Matrix

As previously described, the preferred method for the production of chitosan nanofibers was to dissolve them in acetic before electrospinning. As these samples were not antimicrobial, a number of other acids were also used for dissolving chitosan to form a solution for subsequent electrospinning of the chitosan. Lactic acid, hydrochloric acid and sulphuric acid were attempted for such a process. The greatest success for electrospinning the materials was found when the chitosan materials were dissolved in lactic acid. Lactic acid spun nanofibers of 23.6 g/m² are shown in FIG. 41. However, while using lactic acid provides a material that can be electrospun relatively efficiently, the eventual electrospun spun material was found to form a less organized nanofiber matrix and, also, was found not to be antimicrobial (although it was antibacterial).

Subsequently, a solution was prepared by dissolving chitosan in acetic acid and adding PEO and 10% (w/w) lactic acid. The solution was electrospun and then thermally treated with the resulting nanofibers presented in FIG. 42. The fibers created indicated a more ordered matrix than the matrix formed when the chitosan/PEO fibers solely dissolved in lactic acid. Antimicrobial tests were also performed and positive tests were recorded. These results are presented in FIG. 43.

It can be seen that the acetic acid-lactic acid mixed system creates an antimicrobial Chitosan/PEO product. Therefore this process presents an alternate system to the use of silver to create an antimicrobial product. FIG. 44 and Table 3, below, compare the antimicrobial features of the chitosan/PEO compounds with the various additives discussed above. As summarized, each of the tested chitosan/PEO compounds were tested as sufficiently providing some level of antibacterial, antifungal, and/or antimicrobial compliance, with the exception of the thermally treated chitosan/PEO compound that did not include any further additive compound.

TABLE 3
Summary antibacterial, antifungal and
antimicrobial compliance

| Chitosan/PEO | Anti- | Anti- | Anti- |
|---|---|---|---|
| Product | bacterial | fungal | microbial |
| Non-thermally | Yes | No | No |
| treated | | | |
| Thermally treated | No | No | No |
| Non thermally | Yes | Yes | Yes |
| treated with Ag | | | |
| thermally treated | Yes | No | No |
| with Ag | | | |
| Lactic acid | Yes | No | No |
| dissolved | | | |
| thermally treated | Yes | Yes | Yes |
| Acetic/Lactic acid | | | |

4. Influence of Relative Humidity on Chitosan/PEO Solution

Including the chemical makeup of a specific solution and the ration of the individual compositions that makeup the solution, the operating parameters can also have a potential effect on the resultant electrospun material. The following example discusses the effect of relative humidity on chitosan/PEO electrospun materials.

A chitosan/PEO solution was prepared, with a chitosan solution and a PEO solution being prepared separately and the combined for a final solution. The composition of the final solution is shown below in Table 9.

TABLE 9
Composition of Chitosan/PEO Solution

| | Weight(g) | Weight % | Expected Dry Matter |
|---|---|---|---|
| Chitosan AK | 15 | 5.92 | 15 |
| Demi water | 70.16 | 27.68 | N/A |
| Acetic Acid | 134.86 | 53.21 | N/a |
| 99% | | | |
| PEO | 1.00 | 0.39 | 1.00 |
| Demi water | 32.43 | 12.80 | N/A |
| Total Weight | 253.45 | 100.00 | 16.92 |

The solutions were spun after different periods of being formed. A three day old solution at 30% and 40% relative humidity (RH) was spun, and a ten day old solution at 20%, 30%, 40% RH was spun. The temperature in lab where the spinning took place was held constant at 21° C. In order to observe the impact of the relative humidity to the structure of the spun layer, we didn't use rollers at winding system.

The Electrospinning Equipment was as follows:
Machine: NanoSpider NS-LAB, location Vratislavice, Czech Republic
Electrode: smooth cylinder
Collector: cylinder
Electrode distance: 180 mm
Fabric speed: 0.13 m.min-1
Electrode spin: 4 min-1 HV: 82 kV Number of layer: 5

The results indicated that relative humidity has an influence on the structure of nanofiber layer. It was observed that materials spun at higher relative humidity Had more of a three-dimensional arrangement (i.e. “fluffy”) as compared to the spun materials spun with a lower relative humidity. However, it was also observed that the solutions that were 10 days old appeared to have more of a significant change with respect to the dimensionality of the spun material. As such, while not conclusive, the results indicate that the relative humidity can impact whether the spun material comprises a 2-D or 3-D arrangement.

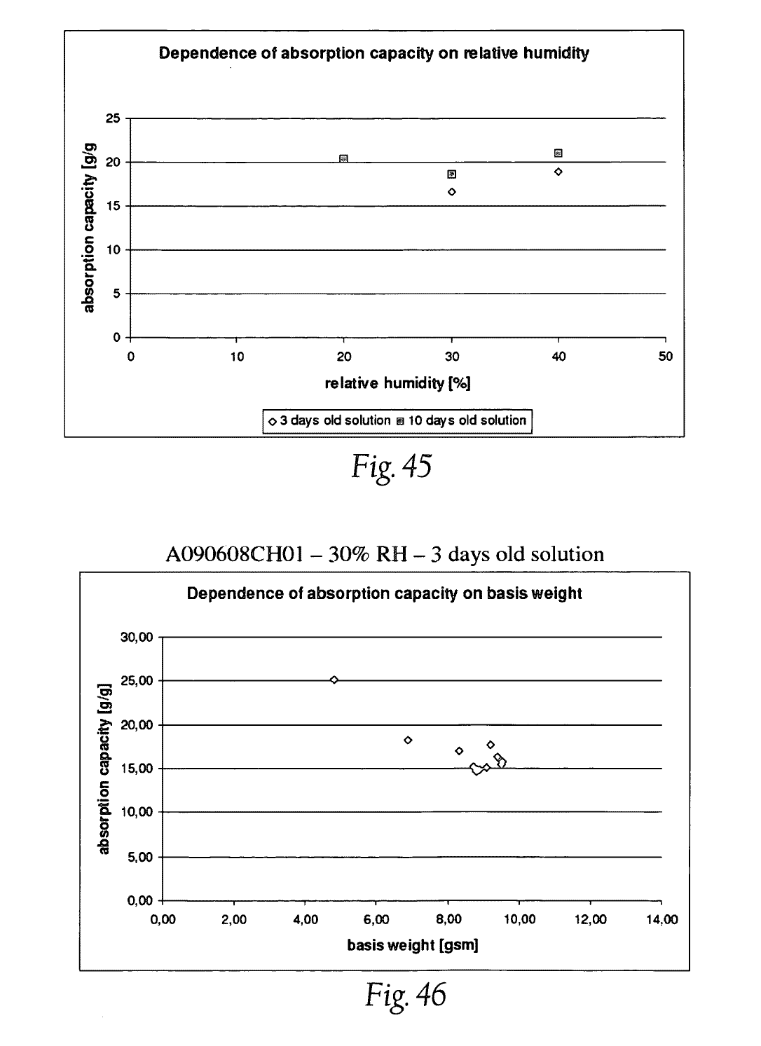

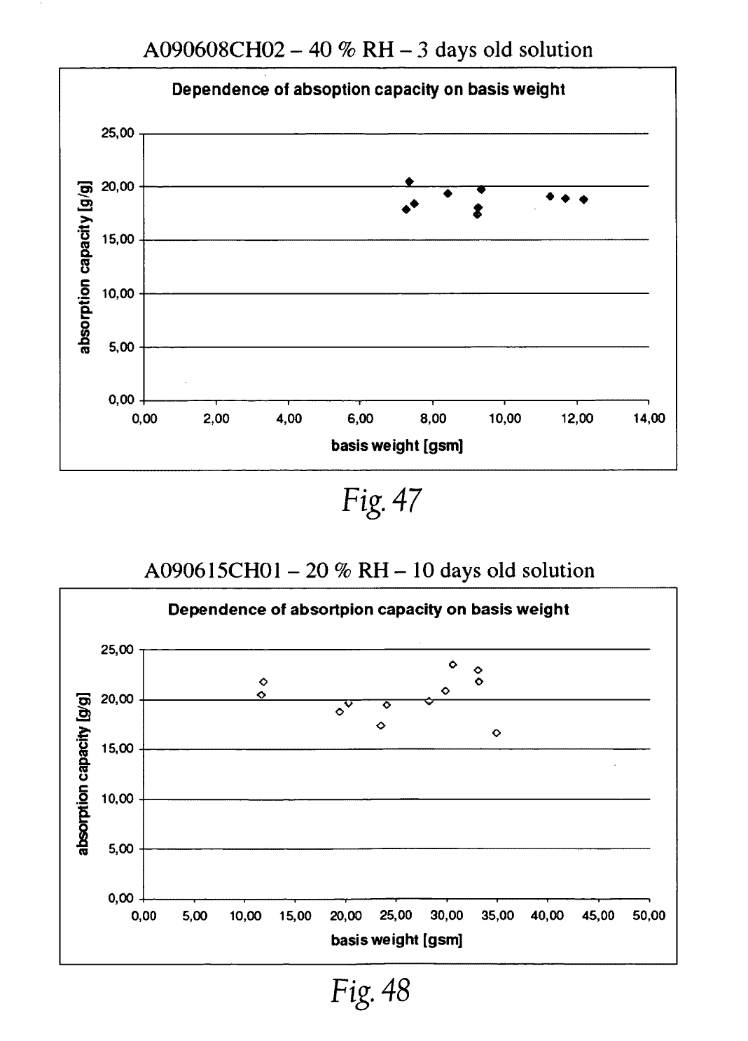

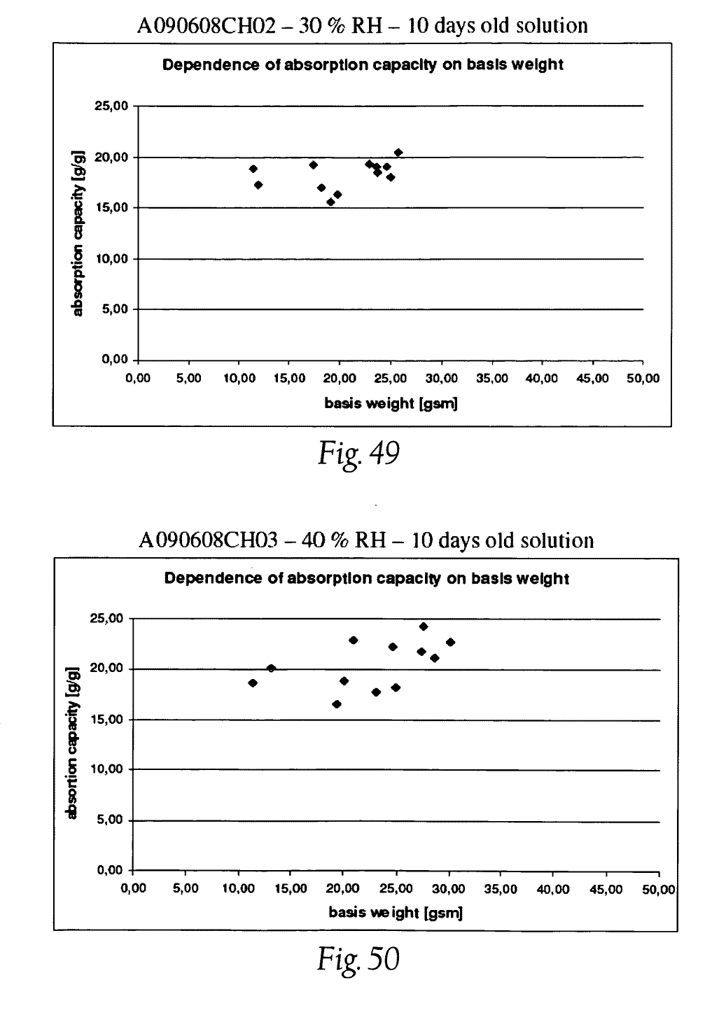

Absorption capacity depends on basis weight and structure of nanofiber layer. The absorption capacity was also analyzed, and the basis weight results are shown in the graphs of FIGS. 45-50. Table 10 shows the fiber diameters of the electrospun materials.

TABLE 10
Fiber Diameter

| Solution Age | RH | Avg.(nm) | Min.(nm) | Max.(nm) |
|---|---|---|---|---|
| 3 day old | 30% | 174 | 60 | 395 |
| 3 day old | 40% | 151 | 60 | 336 |
| 10 day old | 20% | 127 | 53 | 267 |
| 10 day old | 30% | 150 | 53 | 447 |
| 10 day old | 40% | 156 | 59 | 498 |

The initial results, while not conclusive, indicate that the relative humidity may have an effect on whether the electrospun material forms more of a 2-D or a 3-D arrangement. Further, the results indicate that the age of the solution may not have a significant effect on the absorption capacity of the spun material. The following section further describes potential effects on the age of the spun solution.

4. Effects of the Age of the Chitosan/PEO Solution

It has been determined that chitosan/PEO solutions are only capable of being electrospun a certain period of time after preparation. Generally, if the solution is prepared more than about 48 hours prior to the electrospinning process, the solution will lose its spinability, either with a drop of the solutions viscosity or formation of supramolecular agglomerates during storage. Chitosan is more hydrolytically stable than polyethylene oxide, which is known to be hydrolytically unstable. The following section discusses the ability to revitalize older solutions.

A chitosan/PEO solution was prepared by adding 20 grams of chitosan (AK) with 159 g of demineralized water, using a magnetic stirrer (KMO2 (1100)) for approximately 30 minutes. The solution was further shaken for 5 minutes, and then 276.4 g of acetic acid was gradually added. The solution was stirred for an additional 130 minutes. 45.5 g of a 3% PEO solution was further added and stirred for approximately 30 minutes and then allowed to stand for approximately 60 minutes.

The solutions were tested spun with the following parameters:

### Equipment: NanoSpider LAB, Tisnov, Czech Republic

### Electrode: Small Smooth Cylinder

Collector: Needles with spunbond fixed

### Electrode Distance: 130 nm

### Fabric Speed: N/A

Electrode Spin: 3.2 min⁻¹

### HV: 70 or 82 kV

The solutions were attempted to be spun at daily (D) intervals, D=0 and D=4, to determine the effects of age on the spinning. It was determined that after 4 days of storage (D=4), the solutions were not spinnable, as shown in Table 11.

TABLE 11
Comparison of Solution and Process Parameters After 4 Days of Storage

| | Viscosity | Conductivity | T° C. | T° C. | Spun amount after | Loss on Drying | Voltage |
|---|---|---|---|---|---|---|---|
| | (mPa · s) | (mS/cm) | (before) | (after) | 15 min. (g) | (% w/w) | (kV) |
| D = 0 | 495 | 2.44 | 27.1 | 19.0 | 8.7 | 4.25* | 70 |
| D = 4 | 211 | 2.40 | 24.3 | 24.3 | <0.2** | 4.46 | 82 |

*process was cancelled after less than 2 minutes due to sparking
**calculated theoretical value

Four processes four activation of the material were attempted:

1. Mixing the solution with a Turrax stirrer

2. Heating of the solution

3. Heating and mixing with a Turrax stirrer
4. The addition of fresh PEO solution

The results are shown below in Table 12.

TABLE 12
Activation Results

| | Spun amount after | |
|---|---|---|
| Activation | 15 min (g) | Voltage (kV) |
| Mixing by Turrax | Process did not | 82 |
| | start | |
| Heating to 70° C. | Process did not | 82 |
| for 5 min. and | start | |
| cooling to room | | |
| temperature | | |
| Heating for 70° C. | Process did not | 82 |
| while mixing by | start | |
| Turrax | | |
| Addition of 4.5 g | 4.7 | 70 |
| PEO solution (3% | | |
| PEO w/w) to 100 g | | |
| original solution | | |
| Addition of 9.0 g | 8.2 | 70 |
| PEO solution (3% | | |
| PEO w/w) to 100 g | | |
| original solution | | |

The results indicate that reactivation of the solution was capable through the addition of further PEO solution, but heating and stirring did not have an effect on the solution. While possible, it is not certain, however, if the age of the solution will have any effect on the final characteristics of spun materials.

The age of the solution also poses other problems when preparing chitosan solution. For example, acetic acid is often used to prepare the solutions for spinning and to increase the stability of this solution However, this results in a rubberlike substance which has to be mixed for long time to be completely dissolved, usually overnight. As such, the following section discusses whether high-shear mixing (UltraTurrax) could be used for accelerated homogenization of material to minimize issues with stability and spinability in older solutions.

a. Mixing Speeds

A chitosan solution was formed by mixing 10.0 g of chitosan (AK Biotech Co., LTD, China, www.akbio.net.) with 80.0 g of water. The solution was then mixed with 139.0 g of glacial acetic acid, purchased from Fluka Analytcial, in an HDPE flask. Three different solutions were prepared and mixed in three different fashions. The first mixture was mixed using magnetic stirrer, the second by gently mixing using an UltraTurrax 25 mixer and the third vigorously using an UltraTurrax 50 mixer. The two UltraTurrax mixers differ in dispersing elements. The geometry of each, stator and rotor, respectively, are different, as well as diameters of rotor (18 mm and 36 mm, respectively). This results in different shearing forces and different heating during mixing.

The following parameters were used for the electrospinning process:

Equipment: NanoSpider LAB, location TiAnov
Electrode: small smooth cylinder
Collector: needles with spunbond fixed Electrode distance: 130 mm
Fabric speed: −
Electrode spin: 3.2 min⁻¹
HV: variable

After 24 hours, the three solutions were centrifuged (3 min, 3000 min⁻¹) and tested. To determine spinability, 31.8 g of the chitosan solution was first mixed with 3.2 g of 3% PEO in water (w/w, 1.2 g PEO mixed with 38.8 g water) for 30 minutes using magnetic stirrer.

Mixing times and rate are described in Table 13.

TABLE 13
Effect of Mixing

| | Magnetic | | |
|---|---|---|---|
| Mixing Type | Stirrer | UT25 | UT50 |
| Mixing | 1000 | 8000 | 8800 |
| Rate(min⁻¹) | | | |
| Time of Mixing | 150(+120 next | 10 | 7* |
| (min) | day) | | |
| Temperature | 24.9-29.9 | 24.9-31.1 | 24.9-59.4 |
| During Mixing | | | |
| (° C.) | | | |
| Viscosity @ | 367 | 361 | 308 |
| 25° C. (mPa · s) | | | |
| GPC Profile | See FIG. 52 | See FIG. 52 | See FIG. 52 |
| Spinnability | Poor, 3 min. | Good, 12 min. | Poor, 30 sec. |

*Water vapor was observed during UT50 mixing thus UT50 was stopped in 7th minute.

Mixing by magnetic stirrer and UT25 results in almost the same increase of temperature (by 4 and 5° C.) and viscosities of both solutions are also comparable. However, mixing with the UT50 mixer causes high increase of solution temperature (by 35° C.) and viscosity of this solution decreases by about 16%. The effect is shown in FIG. 52, which compares the gel permeation chromatographic (GPC) profile of the chitosan solutions. Chromatograms of “stirrer” and “UT25” are comparable but macromolecular peak (tR=9.4 min) in “UT50” chromatogram is lower. All these solutions were spinnable, however, the best performance of the process was observed for “UT25” solution. Spinability and its performance were only evaluated visually. Either mechanical stress or heating during high-shear mixing could cause decreased viscosity and changes in GPC profile, as such the effects of higher temperature are further explored.

b. The Effect of Temperature Increases

A chitosan solution was formed by mixing 10.1 g of chitosan (AK Biotech) was mixed with 79.5 g of water, which was then mixed with 138.1 g of glacial acetic acid (Fluka) in an HDPE flask. The solution was manually agitated for 5 minutes and mixed by magnetic stirrer for 70 min. Part of this solution was heated in water bath to 71.7° C. for 15 minutes and cooled down do room temperature. The original solution and heated solution were centrifuged (3 min, 3000 min⁻¹) and tested. The electrospinning process had the following parameters:

Equipment: NanoSpider LAB, location Tinov
Electrode: small smooth cylinder
Collector: needles with spunbond fixed Electrode
distance: 130 mm
Fabric speed:
Electrode spin: 3.2 min⁻¹

### HV: 50 kV.

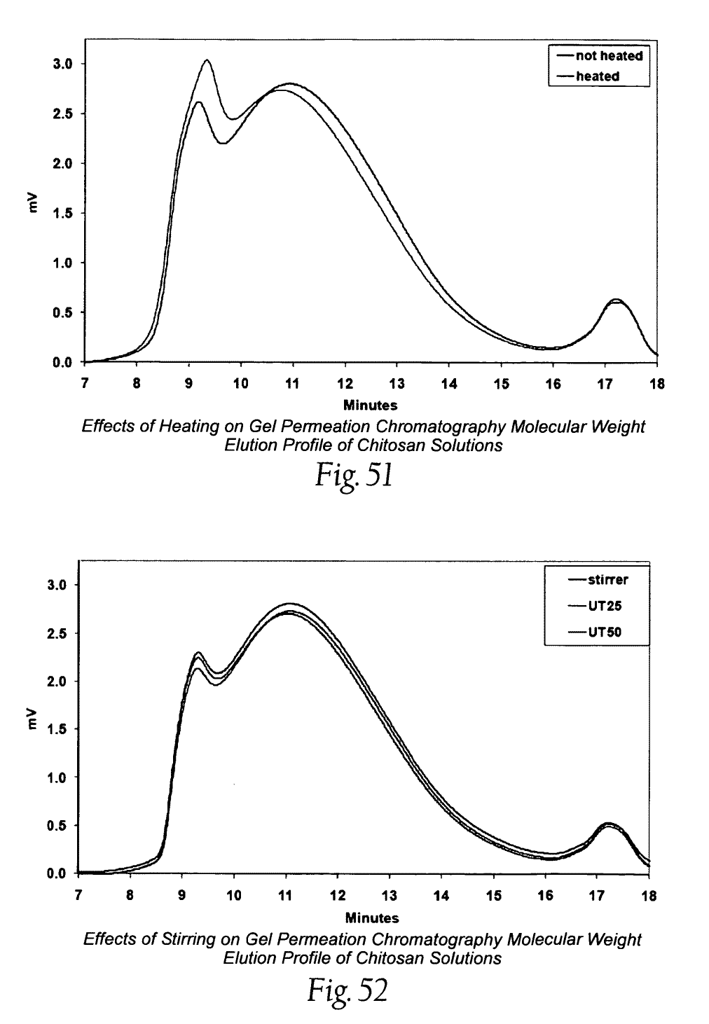

The results indicate that the viscosity of the heated solution (310 mPa·s) was lower by about 36% compared to viscosity of not-heated solution (490 mPa·s). The effect of temperature is also demonstrated in FIG. 51, where the macromolecular peak (tR=9.4 min) is lower for heated solution. It is evident that these macromolecules are transformed to molecules of higher retention (higher peak at tR=10.8 min and shift of peak to higher tR). Both these solutions were spinnable with good and comparable performance. Spinability and its performance were only evaluated visually.

c. Comparison of Chitosan solution with a Chitosan/PEO solution.

The following example compares chitosan solutions and chitosan/PEO solutions with respect to the age of the spun solution.

A chitosan solution was formed by mixing 10.0 g of Chitosan AK with 79.2 g of water and subsequently mixing with 138.0 g of glacial acetic acid (Fluka) in an HDPE flask. This mixture was manually agitated for 5 minutes and mixed by magnetic stirrer for 70 min. A chitosan/PEO solution was prepared similarly to the chitosan solution, with the chitosan solution prepared as stated and then mixed with PEO solution (0.75 g PEO in 24.3 g of water) using magnetic stirrer for 25 minutes. The solutions were stored at room temperature and stored for approximately 24 hours. The solutions were centrifuged (3 min., 3000 min⁻¹) and tested. The electrospinning process had the following parameters.

Equipment: NanoSpider LAB, location Ti\check{s}nov
Electrode: small smooth cylinder
Collector: needles with spunbond fixed
Electrode distance: 130 mm
Fabric speed:
Electrode spin: 3.2 min⁻¹
HV: variable

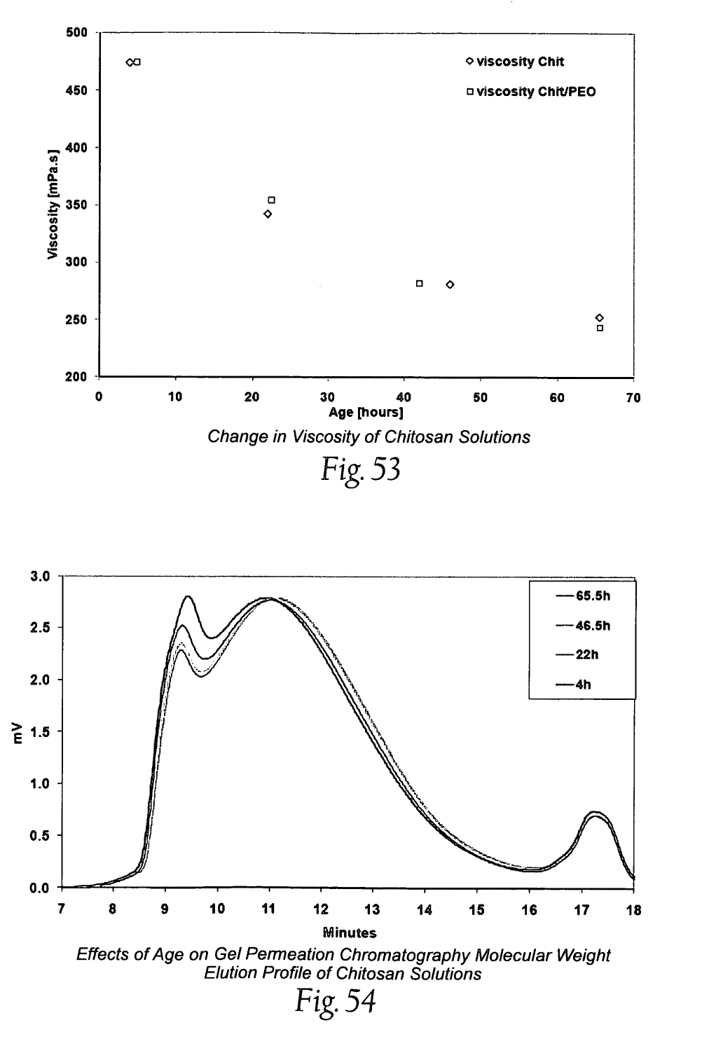

Viscosity was measured, with the results described in Tables 14 and 15 and compared in FIG. 53 for both Chitosan solution and Chitosan/PEO blend. The results of the GPC chromatograms of ageing Chitosan solutions and Chitosan/PEO blends are shown in FIGS. 54 55.

TABLE 14
Ageing of Chitosan solution

Age of Solution (hours)

| | 4 | 22 | 46.5 | 65.5 |
|---|---|---|---|---|
| Viscosity (mPa · s) | 474 | 342 | 281 | 253 |
| GPC Profile | See | Figure | 54 | |
| Spinability | Good | Good | Good | Good |

TABLE 15
Ageing of Chitosan/PEO blend

Age of Solution (hours)

| | 2 | 22.5 | 42 | 65.5 |
|---|---|---|---|---|
| Viscosity (mPa · s) | 474 | 354 | 282 | 244 |
| GPC Profile | See | Figure | 55 | |
| Spinability | Good | Poor | Poor | No |

The viscosity of both the chitosan solution and the chitosan/PEO solution decreased by about 25% during first day of storage at room temperature. The ageing profile of viscosity is virtually the same for both tested mixtures, as shown in FIG. 53. As such, it does not appear viscosity plays a key role in spinability.

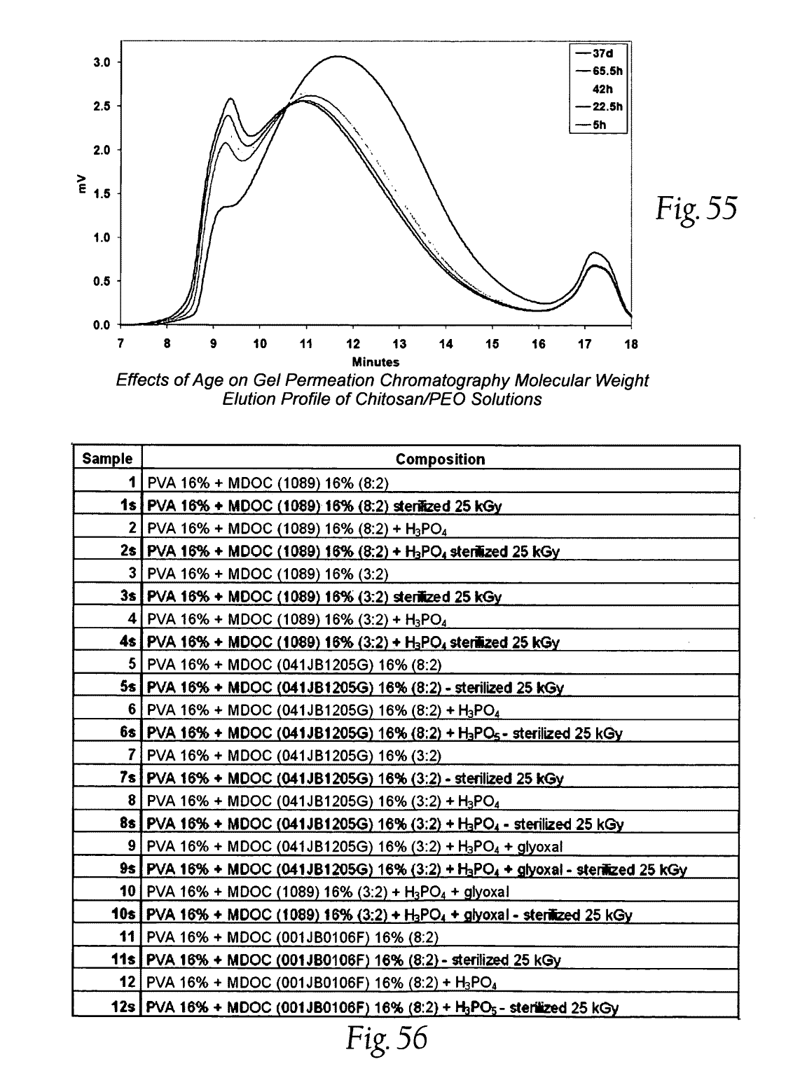

The Gel permeation chromatograph (GPC) shown in FIGS. 54 and 55 indicate a decreasing macromolecular peak (tR=9.4 min) in time and slight shift of the next peak (tR=10.8 min) to higher tR (to the right). This could be due to changes in molecular weight distribution of macromolecules, e.g. hydrolysis of polysaccharide, during storage of chitosan solution.

The chitosan solutions were spinnable when freshly mixed with PEO solution. However, only freshly prepared chitosan/PEO solutions were spinnable. If the chitosan/PEO solution was one or more days old, the spinning process was not satisfactory.

As such, the results indicate that age can have an effect on the spinability of the spun materials.

However, the results do indicate that it is possible to use older solutions for electrospinning in certain situations.

6. Electrospun Chitosan Materials Without PEO

While the focus of the above sections has been on solutions containing chitosan and PEO, the following section discusses electrospinning chitosan solutions without any additional materials. The tested chitosan materials were various chitosan materials, having various levels of deacetylation. The solutions are shown in the Tables 16-20, below.

TABLE 16
Lot. TM3092, Low Molecular Weight
Viscosity of 972 mPa · s @ 24° C.

| | | | Expected dry |
|---|---|---|---|
| Raw material: | Weight (g) | Weight (%) | matter (g) |
| Chitosan Primex | 52.50 | 5.67 | 52.50 |
| Acetic acid 99% | 492.30 | 53.15 | N/A |
| water | 257.80 | 27.83 | N/A |
| 3% PEO in water | 123.66 | 13.35 | 3.71 |
| Total weight | 926.26 | 100.00 | 56.21 |

Electrospinning process:

| | Temperature: 22.7° C. |
|---|---|
| Set parameters | relative humidity: 29% |
| spinning electrode | cylinder |
| collector | cylinder |
| distance between | 15.5 |
| electrodes (cm) | |
| rpm of spinning | 4 |
| electrode | |
| speed of substrate | 10 |
| (cm/min) | |
| type of substrate | PP spunbond |
| applied high voltage | +65/−25 |
| (kV) | |
| number of cycles | 8 |
| achieved basis weight | 20.8 |
| (g/m²) | |
| post-treatment | 130° C./15 min. |

* During the electrospinning process, it was noted that after the 7^{th} cycle process, the viscosity of the solution increased substantially. As such, the electrospinning process was temporarily halted so that the distance between the electrodes could be readjusted to 13.5 cm for continuing electrospinning.

TABLE 17
Lot. SA 3009, 22012
Viscosity of 344 mPa · s @ 25° C.

| | | | Expected dry |
|---|---|---|---|
| Raw material: | Weight (g) | Weight (%) | matter (g) |
| Chitosan Aliquot | 21.0 | 4.07 | 21.00 |
| Acetic acid 99% | 257.0 | 49.75 | N/A |
| water | 143.0 | 27.68 | N/A |
| 3% PEO in water | 95.6 | 16.50 | 2.87 |
| Total weight | 516.6 | 100.00 | 23.87 |

Electrospinning process:

| | Temperature: 25° C. |
|---|---|
| Set parameters | relative humidity: 33.3% |
| spinning electrode | cylinder |
| collector | cylinder |
| distance between | 15.5 |
| electrodes (cm) | |
| rpm of spinning | 4 |
| electrode | |
| speed of substrate | 5 |
| (cm/min) | |
| type of substrate | PP spunbond |
| applied high voltage | +70/−30 |
| (kV) | |
| number of cycles | 8 |
| achieved basis weight | 24 |
| (g/m²) | |
| post-treatment | x |
| date of experiment | 1.7.2008 |

*The electrospinning process above obtained planar, i.e. flat, layers of nanomaterial.

TABLE 18
Lot RD080811, Degree of Deacetylation-60-65%,
Viscosity of 790 mPa · s @ 28° C.

| | | | Expected dry |
|---|---|---|---|
| Raw material: | Weight (g) | Weight (%) | matter (g) |
| Chitosan 19 mPas | 20.0 | 3.71 | 20.00 |
| Acetic acid 99% | 307.5 | 57.10 | N/A |
| water | 161.0 | 29.90 | N/A |
| 3% PEO in water | 50.0 | 9.29 | 1.50 |
| Total weight | 538.5 | 100.00 | 21.50 |

Electrospinning process:

| | Temperature: 27° C. |
|---|---|
| | relative humidity: |
| Set parameters | 23% |
| spinning electrode | cylinder |
| collector | cylinder |
| distance between | 15.5 |
| electrodes (cm) | |
| rpm of spinning | 4 |
| electrode | |
| speed of substrate | 5 |
| (cm/min) | |
| type of substrate | PP spunbond |
| applied high voltage | +65/−27.5 |
| (kV) | |
| number of cycles | 4 |
| achieved basis weight | 30.8-33.2 |
| (g/m²) | |
| post-treatment | 130° C./30 min. |
| date of experiment | 18.9.2008 |
| spinning electrode | cylinder |
| collector | cylinder |
| distance between | 15.5 |
| electrodes (cm) | |
| rpm of spinning | 4 |
| electrode | |
| speed of substrate | 5 |
| (cm/min) | |
| type of substrate | PP spunbond |
| applied high voltage | +65/−35 |
| (kV) | |
| number of cycles | 4 and 4 |
| achieved basis weight | 24 |
| (g/m²) | |
| post-treatment | 130° C./30 min. |
| date of experiment | 1.7.2008 |

*The material was spun so that it resulted in a 3-D arrangement, i.e. a fluffy material. The material was also spun with the parameters below to achieve a planar layers of nanomaterial.

TABLE 19
Lot RD0808012, Degree of Deacetylation 65-70%,
Viscosity of 838 mPa · s @ 26° C.

| | | | Expected dry |
|---|---|---|---|
| Raw material: | Weight (g) | Weight (%) | matter (g) |
| Chitosan 19 mPas | 20.0 | 3.13 | 20.00 |
| Acetic acid 99% | 367.5 | 57.56 | N/A |
| water | 201.0 | 31.48 | N/A |
| 3% PEO in water | 50.0 | 7.83 | 1.50 |
| Total weight | 638.5 | 100.00 | 21.50 |

| | Temperature: 27° C. |
|---|---|
| | relative humidity: |
| Set parameters | 23% |
| spinning electrode | cylinder |
| collector | cylinder |
| distance between | 15.5 |
| electrodes (cm) | |
| rpm of spinning | 4 |
| electrode | |
| speed of substrate | 10 |
| (cm/min) | |
| type of substrate | PP spunbond |
| applied high voltage | +65/−30 |
| (kV) | |
| number of cycles | 4 |
| achieved basis weight | 5.4-8.3 |
| (g/m²⁾ | |
| post-treatment | 130° C./30 min. |
| date of experiment | 19.9.2008 |

* The electrospinning process resulted in a 3-D material, having a very compact layer of nanomaterial. 2-D materials were not produced with this material.

TABLE 20
Lot RD080814, 60-65% Degree of
Deacetylation, Viscosity unknown

| | | | Expected dry |
|---|---|---|---|
| Raw material: | Weight (g) | Weight (%) | matter (g) |
| Chitosan 19 mPas | 8.0 | 1.62 | 8.0 |
| Acetic acid 99% | 303.0 | 61.16 | N/A |
| water | 184.4 | 37.22 | N/A |
| Total weight | 495.4 | 100.00 | 8.0 |

* The sample of Table 20 was not spun, as the viscosity was believed as being too high for spinning purposes.

### CONCLUSION

The results indicate that chitosan materials, without the addition of PEO, are capable of being electrospun, and are also capable of forming both planar and 3-D layers of nanomaterials.

C. Electrospun Microdispersed Oxidized Cellulose (MDOC)/Polyvinyl alcohol (PVA) Matrix

Electrospun microdispersed oxidized cellulose (MDOC) compounds were tested and compared to assess the quantitative and qualitative characteristics of the MDOC compounds. Particularly, the compounds tested were a composition of MDOC and polyvinyl alcohol (PVA) with additives added to the MDOC/PVA matrix. Tested additives included phosphoric acid, glyoxal, and perphosphoric acid. The compositions were also tested with and without sterilization. The various tested compositions are shown in FIG. 33. The phosphoric acid used was at a strength of 85%-8 wt. % related on the dry mass of basic mixture (PVA+MDOC), and the glyoxal used was at a strength of 40%-10 wt. % related on the dry mass of basic mixture (PVA+MDOC). The electrospinning parameters are shown below in Table 21.

TABLE 21
Electrospinning Parameters for MDOC/PVA
Matrices

| | |
|---|---|
| Method | static |
| Distance between | 10 cm |
| electrodes | |
| Voltage | 55 kV |
| Humidity | 40% RH |
| Temperature | 22° C. |
| Air stream | no |

The compositions were tested and compared to determine how the noted additives affected the MDOC/PVA matrix. The results are discussed in the following sections. For reference, the number of the samples in the legends of the following Figures refers to the number of the compound as listed in FIG. 57. For example, if a legend in the following graphs and figures may refer to compound “1”. This would be sample 1 of FIG. 57, PVA+MDOC in an 8:2 ratio.

1. Influence of Phosphoric Acid on an Insoluble MDOC/PVA Matrix (8:2 Ratio)

The effect of phosphoric acid on the strength and elongation of the MDCO/PVA matrix was tested. Matrixes 1 and 2 from FIG. 33 were compared against one another, both with and without sterilization. The MDOC material is an insoluble material. The results are shown in FIG. 58, comparing the strengths of the nanofibrous matrices, and FIG. 59 comparing the elongation of the matrices.

While not conclusive, initial results indicated that the presence of phosphoric acid potentially increased the strength of the matrices and did not effect the elongation of the matrices. Strength also increased with increasing basis weight.

Similarly, there results indicated that that sterilization potentially increased the strength of the tested matrices, but the suggested correlation may be based on other factors. However, elongation of the matrices was lower after gamma-irradiation sterilization of the matrices.

It should also be noted that elongation of the matrices increases with decreasing basis weight. For example, in such uses, such as using the nanospun materials in plasters, a basis weight of around 10 g/m² is preferred, while still maintaining elongation for the matrix.

2. Influence of Phosphoric Acid and Glyoxal on the MDOC/PVA Matrix (3:2 Ratio)

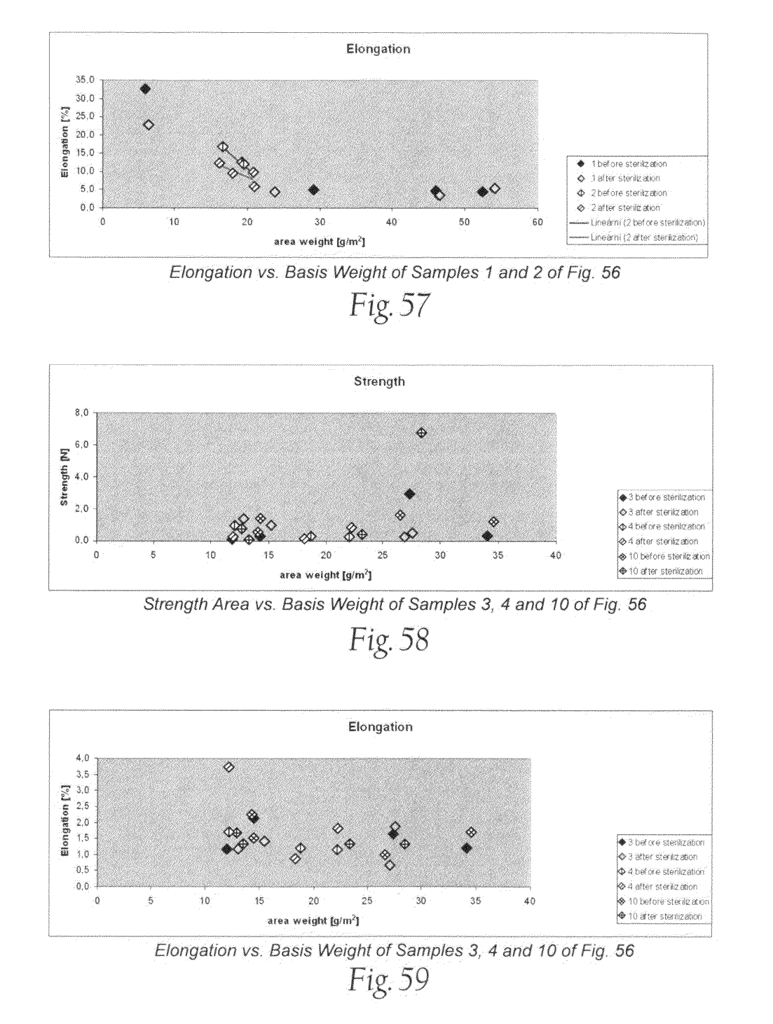

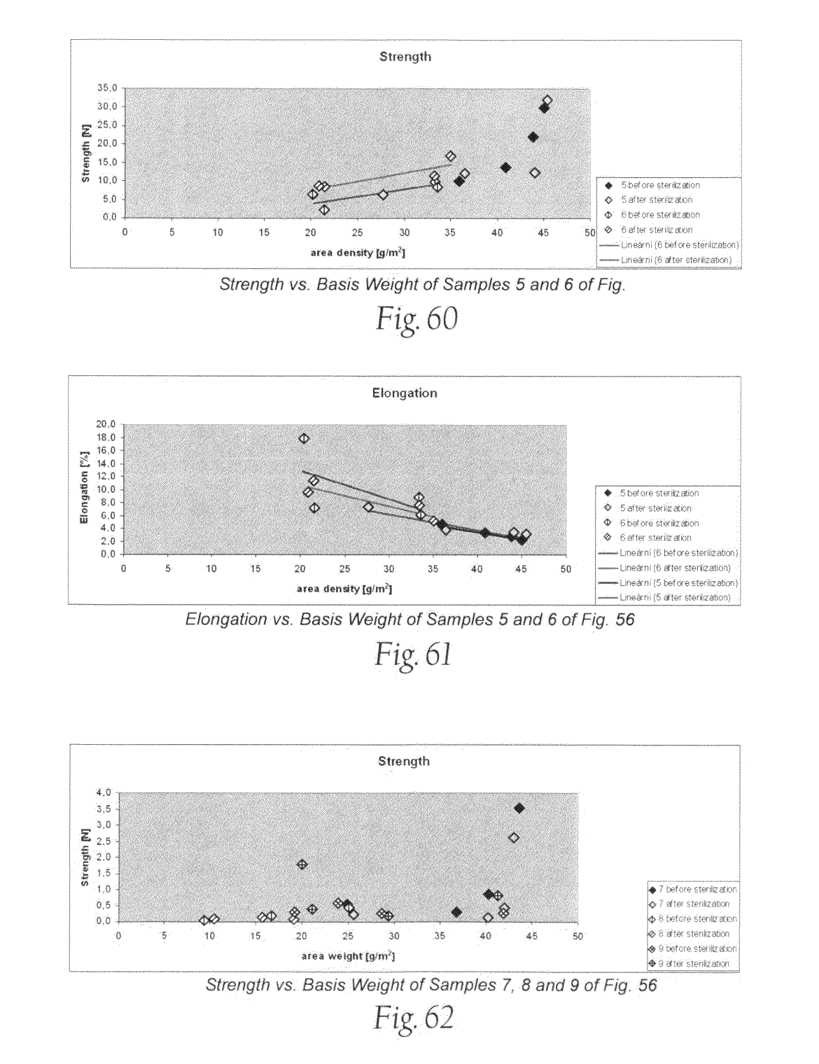

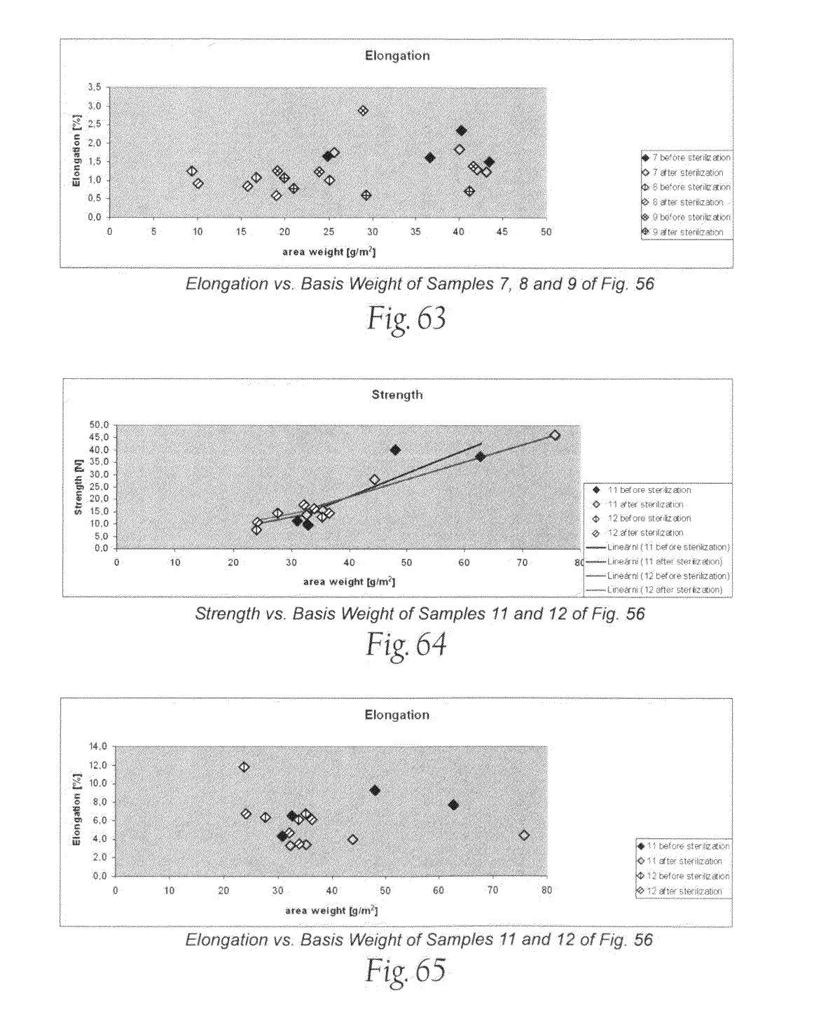

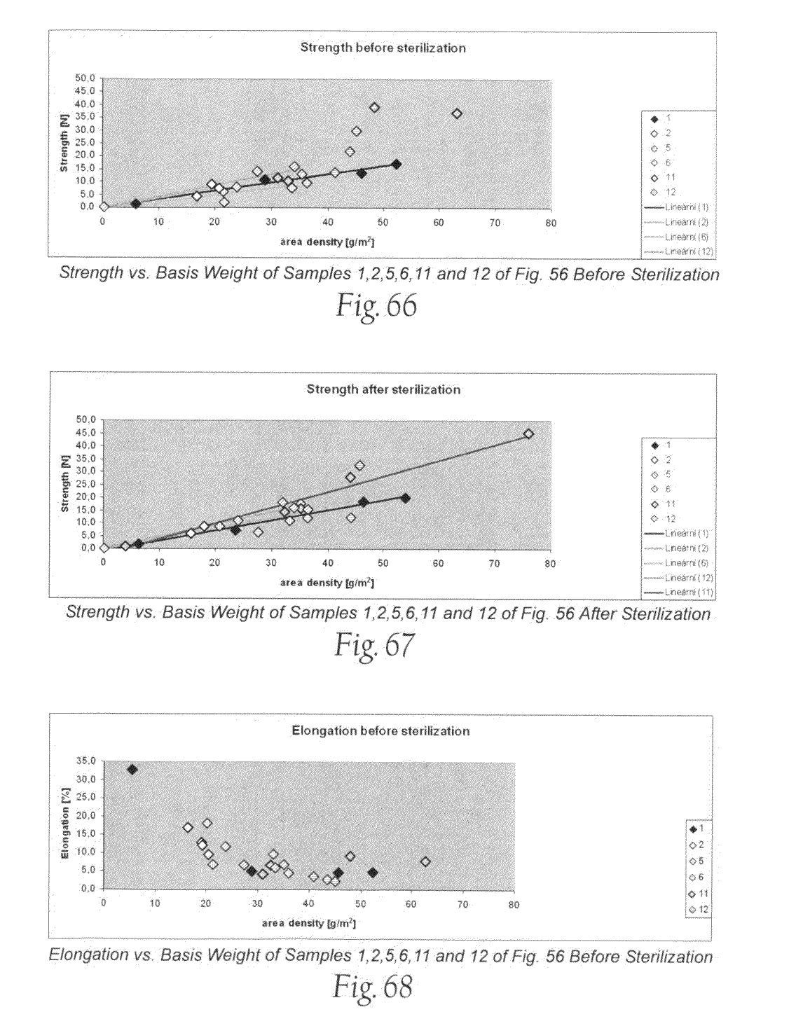

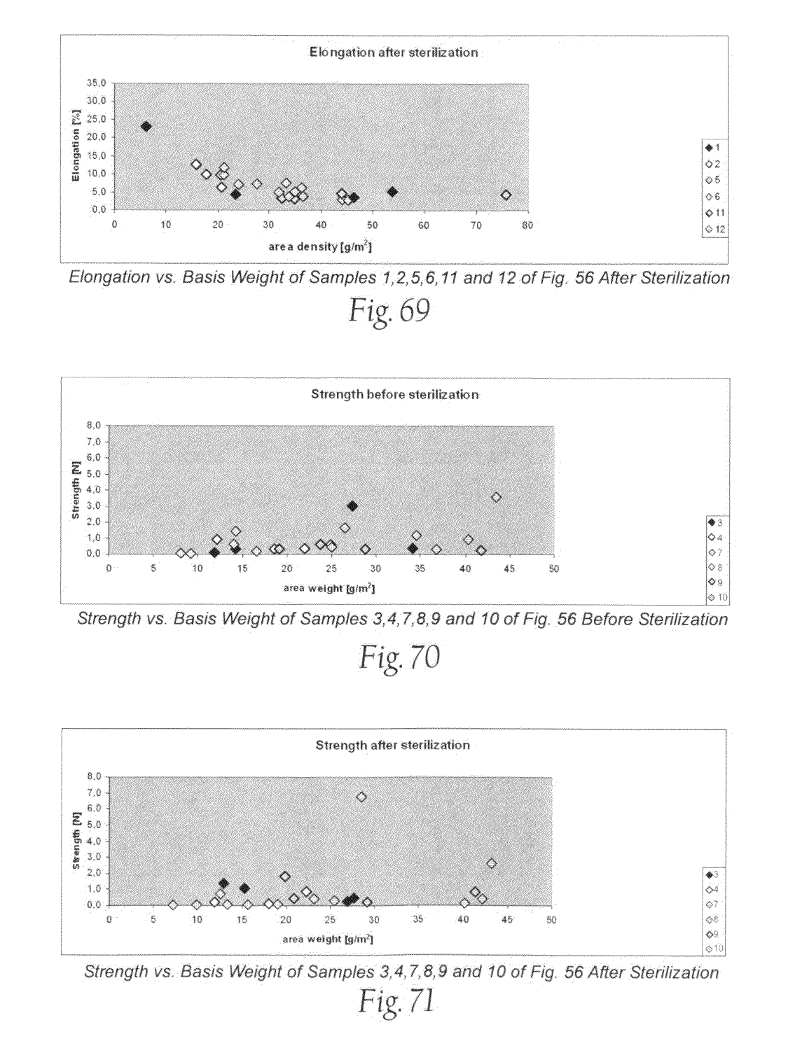

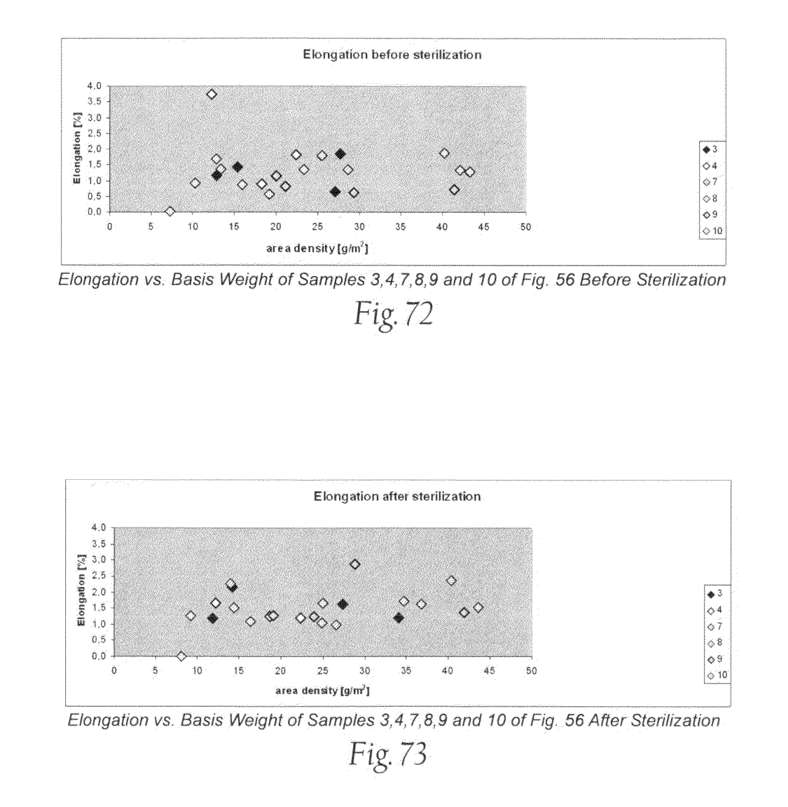

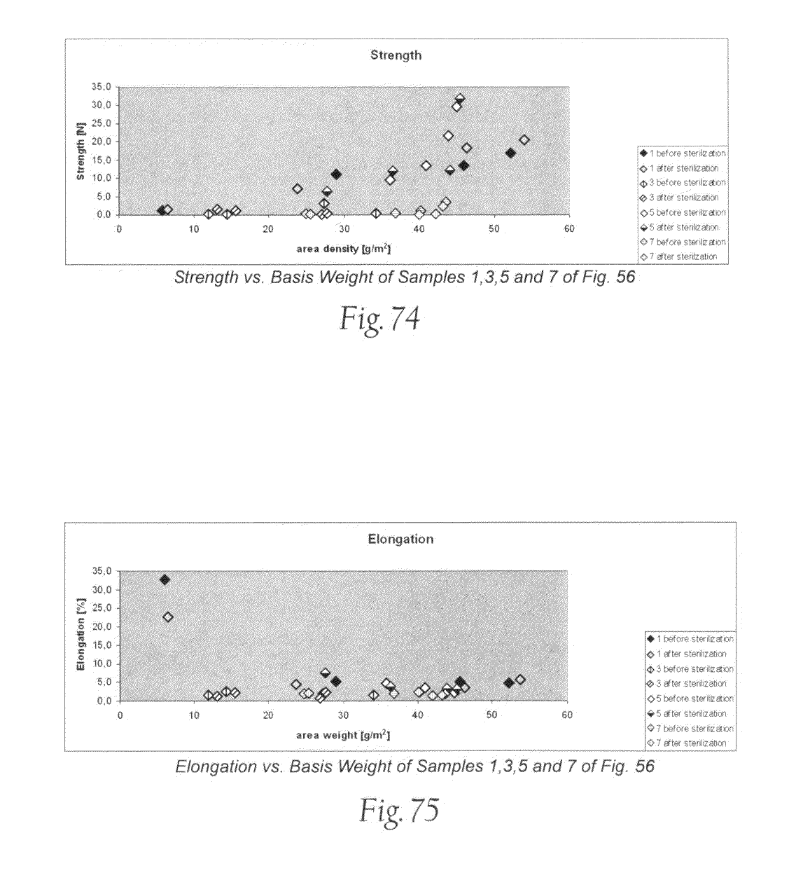

The effect of the addition of phosphoric acid and glyoxal on the strength and elongation of the MDCO/PVA matrix was tested. Matrixes 3, 4, and 10 from FIG. 56 were compared against one another, both with and without sterilization. Matrixes 3 and 4 had phosphoric acid added, while Matrix 10 had both phosphoric acid and glyoxal added. The results are shown in FIG. 59, comparing the strengths of the nanofibrous matrices, and FIG. 60 comparing the elongation of the matrices.

The results for both strength and elongation show no discernible trends, and the addition of either phosphoric acid or glyoxal does not affect the mechanical properties significantly. Because of the weak correlation, it was not possible to determine the positive or negative effect that gamma-irradiation sterilization would have on the matrices.

3. Influence of Phosphoric Acid on a Soluble MDOC/PVA Matrix (8:2 Ratio)

The effect of phosphoric acid on the strength and elongation of the MDOC/PVA matrix was tested. Matrixes 5 and 6 from FIG. 56 were compared against one another, both with and without gamma-irradiation sterilization. It should be noted that the MDOC compound used in matrices 5 and 6 is a soluble compound compared to the insoluble MDOC compound of matrices 1 and 2, tested above. The results are shown in FIG. 61, comparing the strengths of the nanofibrous matrices, and FIG. 62 comparing the elongation of the matrices.

The results indicated that the presence of phosphoric acid increased the strength of the sterilized samples, while the strength was not changed significantly by the addition of phosphoric acid for nonsterilized samples. Similar to the previously tested insoluble MDOC matrices, elongation increases with decreasing basis weight.

Strength also increases with increasing basis weight, with the increase being non-linear, and was also increased after sterilization. However, the addition of phosphoric acid indicated that elongation was better prior to sterilization. Sterilization did not appear to have an effect on the matrices that did not contain phosphoric acid.

4. Influence of Phosphoric Acid and Glyoxal on Strength of a Soluble MDOC/PVA Matrix (3:2 Ratio)

The effect of the addition of phosphoric acid and glyoxal on the strength and elongation of the MDCO/PVA matrix was tested. Matrixes 7, 8, and 9 from FIG. 56 were compared against one another, both with and without sterilization. Matrixes 7 and 8 had phosphoric acid added, while Matrix 9 had both phosphoric acid and glyoxal added. The results are shown in FIG. 63 comparing the strengths of the nanofibrous matrices, and FIG. 64 comparing the elongation of the matrices. It should be noted that the MDOC compound used in matrices 11 and 12 is a soluble compound compared to the insoluble MDOC compound of matrices 1 and 2, tested above.

Similar to the previously tested insoluble MDOC matrices, the matrices had weak mechanical properties. Strength of the samples appears to increase with increasing basis weight and the trend is non linear. Strength was not changed significantly for both sterilized and non sterilized matrices by addition of phosphoric acid and phosphoric acid and glyoxal.

Elongation of all matrices (7, 8, and 9) is lower after sterilization which means that sterilization worsens the elongation of the matrices. Elongation of the basic mixture without phosphoric acid and glyoxal is higher than elongation of the mixture with mentioned additives, meaning that, in this case, additives have negative influence on elongation and no effect on strength. There is no supposition of any trend of elongation, which may have a connection with difficult measurability.

5. Influence of Phosphoric Acid on Strength and Elongation of a Soluble MDOC/PVA Matrix (8:2 Ratio)

The effect of phosphoric acid on the strength and elongation of the MDOC/PVA matrix were further tested. Matrixes 11 and 12 from FIG. 56 were compared against one another, both with and without gamma-irradiation sterilization. It should be noted that the MDOC compound used in matrices 11 and 12 is a soluble compound compared to the insoluble MDOC compound of matrices 1 and 2, tested above. The results are shown in FIG. 65, comparing the strengths of the Nan fibrous matrices, and FIG. 66 comparing the elongation of the matrices.

The initial results, while not conclusive, indicate that the presence of phosphoric acid likely did not increase the strength of the sterilized samples, and the strength was not changed significantly by the addition of phosphoric acid for nonsterilized samples. Similar to the previously tested insoluble MDOC matrices, elongation appears increases with decreasing basis weight.

However, the addition of phosphoric acid indicated no effect on elongation prior to sterilization. Sterilization appeared to have a negative effect on the elongation of the samples, but did not appear to follow any trend.

6. Effects of Other Acids on the MDOC/PVA Matrix

PVA and PVA-based nanofiber material can be made insoluble in water by means of heat treatment of material containing phosphoric acid. This methodology has already been used in Vratislavice Lab for cross-linking of MDOC:PVA nanofiber samples. The cross-linking mechanism of PVA with polyfunctional acids is well known. Esters are formed between hydroxyl groups of PVA and acidic groups of polyfunctional acids during heat treatment, resulting in cross-linking. With boric acid, this cross-linking can even be observed in aqueous solution at room temperature.

The following experiment assesses the applicability of other acids and the validity of potential cross-linking mechanisms of these acids, since phosphoric acid could be undesirable for wounds and skin.

The experiment assessed various types of acids, including: hydrochloric acid (strong inorganic monofunctional acid), acetic acid (weak organic monofunctional acid), phosphoric acid (inorganic trifunctional acid), citric acid (weak organib trifunctional hydroxy-acid), lactic acid (weak organic monofunctional hydroxy-acid) and 1,2,3,4-butanetetracarboxylic acid (weak organic tetrafunctional acid). The results are shown in section a, below. Section b, below further discussed the cross-linking of PVA:Citric acid blend, and the effect of acid concentration on the solution for electrospinning. Section c, below, discussed the use of MDOC-H+ as a polyfunctional acid, which could be used instead of phosphoric acid in cross-linking of MDOC:PVA nanofibers.

a. PVA/MDOC Combine With Various Acids

A polyvinyl alcohol solution (PVA) (16±1.5) % m/m, was purchased from Novacke chemicke zavody, Slovakia. The PVA solution was mixed with a predetermined amount of an acid compound (see Table 22), a MDOC compound (batch No. X01032), and deionized water using an UltraTurrax T25 mixture. The ratio of MDOC:PVA in the solution was maintained at 4:6 (unless MDOC was added to a specific solution. Approximately 25 g of each tested solution were placed into a spinning bath and electrospun for 15 minutes under the following conditions:

Equipment: NanoSpider LAB, location Ti\check{s}nov
Electrode: small cylinder
Collector: Wire with spunbond fixed
Fabric speed: 0, static spunbond
Electrode spin: 3.2 min⁻¹

The formed layers of nanospun material were then treated at 140° C. for 2 hours, with the results shown in Table 22.

TABLE 22
MDOC:PVA Solution Treated with Acid Solutions

| | Acid | | | | | | | | | |
|---|---|---|---|---|---|---|---|---|---|---|
| | concen- | | | | | Acid | | | | |
| | tration | | | | | content | | | Amount | |
| | (original | | | | | in dry | | Conduc- | of spun | |
| | chemical) | MDOC | water | PVA | acid | matter | pH of | tivity | material | Behavior of sample after immersion in water |
| Acid | [% w/w] | [g] | [g] | [g] | [g] | [% w/w] | blend | [mS/cm] | [g] | (immediately after sample production) |
| Citric | 100 | 0 | 5.942 | 44.01 | 0.564 | 7.4 | 2.64 | 1.08 | 16.2 | contraction of dimensions, wetting, no |
| | | | | | | | | | | swelling, no dissolution |
| Butane- | 100 | | 5.908 | 43.88 | 0.560 | 7.4 | NA | 0.742 | 3.8 | contraction of dimensions, wetting, no |
| tetra- | | | | | | | | | | swelling, no dissolution |
| carboxylic | | | | | | | | | | |
| Lactic | 100 | | 5.836 | 43.78 | 0.560 | 7.4 | NA | 0.928 | 8.6 | almost immediate dissolution |
| Phosphoric | 84 | | 0 | 49.41 | 0.767 | 7.5 | 1.67 | 6.21 | 5.4 | swelling, no changes in dimensions, no |
| | | | | | | | | | | dissolution |
| Hydrochloric | 35 | | | 49.82 | 0.412 | 1.8 | 1.27 | 18.69 | 3.2 | swelling (gel), slow decay, no dissolution |
| Acetic | 99 | | | 48.99 | 1.065 | 11.9 | 3.29 | 0.712 | 12.3 | swelling (gel), decay, dissolution in few |
| | | | | | | | | | | minutes |
| Phosphoric | 84 | 3.163 | 16.64 | 29.6 | 0.779 | 7.6 | 3.09 | 7.31 | 4.7 | swelling, rolling, no dissolution |
| Hydrochloric | 35 | 3.190 | 16.73 | 29.9 | 0.395 | 1.7 | 3.42 | 8.66 | 4.8 | swelling (opaque gel), no dissolution, |
| | | | | | | | | | | resistant for 1 week |
| Acetic | 99 | 3.131 | 16.50 | 29.37 | 1.005 | 11.3 | 3.84 | 5.68 | 6.5 | swelling (opaque gel), decay after 1 week |

The results indicate that the addition of acid to the PVA solutions strongly influences conductivity of solution and pH value. In the MDOC/PVA solutions, the pH values appear to be buffered by the MDOC materials in the solutions.

The results further indicate that the PVA non-treated nanofiber samples were completely soluble in water. nanofiber samples, containing tri- and tetrafunctional acids (phosphoric, citric, butanetetracarboxylic) and heated to 140° C. for 2 hours, were not soluble in water, which suggests a potential cross-linking of the materials within the solutions. Heat treated nanofiber samples, containing only weak organic monofunctional acids (acetic, lactic), were soluble in water, denoting no significant change in structure (no cross-linking). The sample with hydrochloric acid after heat treatment was not soluble in water, which indicates some change in chemical structure, but the mechanism is not certain.

The MDOC/PVA nanofiber samples not heat treated were completely soluble in water, resulting in opaque solution (free H+ form of MDOC is water-insoluble). All other non-treated samples were insoluble, resulting in opaque gel.

The results indicate that there is potential to replace certain acids currently used in electrospinning processes with other acids that may have less deleterious effects when eventually used in wound dressing assemblies. Initial results indicate that bi-, tri- and poly-functional acids can be used for cross-linking of PVA based nanofiber materials, with the formation of esters between hydroxyl groups of PVA and acidic groups of acid believed to be the main mechanism of cross-linking. As such potential cross-linking exists, the following sections discuss in further detail solutions comprising citric acid.

b. Effect of Citric Acid Concentration on the Cross-Linking with PVA

As stated above, it is contemplated that citric acid can be a more suitable alternative to H₃PO₄ in MDOC/PVA solutions. The MDOC/PVA and PVA solutions were formed as noted in section (a). Citric acid was added to the various samples, as stated in Table 23.

Approximately 25 g of each sample were placed into spinning bath and electrospun for 15 minutes under the following conditions:

Equipment: NanoSpider LAB, location Ti\check{s}nov
Electrode: small cylinder
Collector: wire with spunbond fixed
Fabric speed: 0, static spunbond
Electrode spin: 3.2 min⁻¹

Layers of nanospun material were then treated at 140° C. for 2 hours. The results are shown in Table 23.

TABLE 23
MDOC/PVA/Citric Acid Solutions

| | Acid | | | | | | | | | |
|---|---|---|---|---|---|---|---|---|---|---|
| | concen- | | | | | Acid | | | | |
| | tration | | | | | content | | | Amount | |
| | in original | | | | | in dry | | Conduc- | of spun | |
| | chemical | MDOC | water | PVA | acid | matter | pH of | tivity | material | Behavior of sample after immersion in water |
| Acid | [% w/w] | [g] | [g] | [g] | [g] | [% w/w] | blend | [mS/cm] | [g] | (immediately after sample production) |
| Citric | 100 | 0 | 5.801 | 43.77 | 0.561 | 7.5 | NA | 1.076 | 10.2 | contraction, wetting, no swelling, no |
| | | | | | | | | | | dissolution, least elastic |
| | | | 6.014 | 43.79 | 0.280 | 3.9 | | 0.752 | 8.3 | contraction, wetting, no swelling, no |
| | | | | | | | | | | dissolution, less elastic |
| | | | 6.319 | 43.71 | 0.140 | 2.0 | | 0.577 | 9.0 | contraction, wetting, no swelling, no |
| | | | | | | | | | | dissolution, elastic |
| | | | 6.400 | 43.8 | 0.071 | 1.0 | | 0.536 | 9.7 | contraction of dimensions, swelling, dissolution |
| | | | 6.301 | 43.71 | 0.035 | 0.5 | | 0.545 | 10.4 | decay, dissolution¹⁾ |
| | | 2.804 | 20.444 | 26.24 | 0.560 | 7.5 | | 5.14 | 7.9 | wetting, swelling (gel), no decay, rolling |
| | | 2.794 | 20.680 | 26.35 | 0.281 | 3.9 | | 4.89 | 8.2 | wetting, swelling (gel), no decay |
| | | 2.789 | 20.844 | 26.49 | 0.141 | 2.0 | | 4.78 | 8.7 | welting, swelling (gel), only slow decay |
| | | 2.784 | 20.973 | 26.27 | 0.071 | 1.0 | | 4.76 | 7.8 | wetting, swelling (gel), decay in 20 minutes |
| | | 2.809 | 21.284 | 26.44 | 0.035 | 0.5 | | 4.77 | 8.3 | wetting, swelling (gel), decay in 15 minutes |

The results indicate that by altering the amount citric acid in the final PVA material (0.5-7.5%), the properties of the resultant nanofiber layer can also be altered. Samples with higher citric acid content (more than 2%) appeared to be insoluble in water, did not swell to gel, and had a high tensile strength and elongation in wet form (which was evaluated only manually/visually). Samples with lower citric acid content were soluble or easily decayed.

The samples containing MDOC swells into a gel when immersed in water and again, higher content of acid in the resultant layer reflects in better stability in water. However, while there is an initial indication of cross-linking within the samples, it is possible that the cross-linking agent could be either citric acid or poly-anhydroglucuronic acid (PAGA), which is produced by reaction of the Na/Ca salt (MDOC) with citric acid. Further, Heat treatment of PVA:Citric acid nanofibers results in material, which is not soluble in water, do not swell and exhibits apparently high tensile strength and elongation in wet form.

b. PVA Cross-Linking with MDOC-H+

The results above indicate that MDOC compounds could have potential cross-linking abilities. Assessment was carried out using MDOC-H+ (free poly-anhydroglucuronic acid (PAGA)) on PVA cross-linking, so that the PAGA would not interfere with the potential results. Because MDOC material is organic as opposed to inorganic, this polyfunctional acid in pure form could be used for cross-linking PVA based materials with its organic nature instead of inorganic (phosphoric) or any other “extrinsic” acids, e.g. in blend with regular. Na/Ca MDOC salt. Limited solubility of MDOC-H+ could affect electrospinning process and especially the microscopic quality of nanofiber layer (macro- and micro-defects). Water-soluble ammonium salt (MDOC-NH4) could be used instead of MDOC-H+. Solubility of this salt could avoid formation of macro and micro defects. Ammonium should dissociate from MDOC-NH₄ molecule at higher temperature (heat treatment) forming free acid for cross-linking reaction.

A PVA solution (16±1.5) % m/m was purchased from Novacke chemicke zavody, Slovakia. MDOC-H+ (batch No. 012H+0909) was prepared by dissolving original oxidized cellulose (Synthesia) in sodium hydroxide, which was then re-precipitated into free H+ form by adding nitric acid and washing off residuals. MDOC-H+ was then dried and micronized. The PVA solution was mixed with various amounts of MDOC-H+ as noted in Table 24 (which was previously swollen in water for 16 hours), using UltraTurrax T25. The solutions were centrifuged to remove any undissolved particles. The MDOC-NH₄ salt solution was prepared by adding concentrated ammonia into MDOC-H+ suspension (approx 0.4 ml for complete dissolution of 0.56 g MDOC-H+) before mixing with the PVA.

Approximately 25 g of each solution were placed into spinning bath and electrospun for 15 minutes under conditions described below:

Equipment: NanoSpider LAB, location Ti\check{s}nov
Electrode: small cylinder
Collector: wire with spunbond fixed
Electrode distance: 160 mm
Fabric speed: 0, static spunbond
Electrode spin: 3.2 min⁻¹

### HV: 75 kV

Layers of nanospun material were then treated at 140° C. for 2 hours. The results are shown below in Table 24.

TABLE 24
Preparation of MDOC-H+:PVA Solutions and behavior of nanofiber samples in water

| | | | | Acid | | | |
|---|---|---|---|---|---|---|---|
| | | | | content | Amount | | |
| | | | | in dry | of spun | | |
| | water | PVA | acid | matter | material | Macro | Behavior of sample after immersion in water |
| Acid/salt | [g] | [g] | [g] | [% w/w] | [g] | defects | (immediately after sample production) |
| MDOC-H+ | 6.130 | 43.808 | 0.140 | 2.1 | 9.1 | 1 | swelling (gel), dissolution |
| | 5.738 | 43.847 | 0.561 | 7.9 | 9.2 | 2 | swelling (opaque gel), stable |
| | 4.872 | 43.782 | 1.400 | 17.6 | 8.5 | 4 | swelling (more dense opacity of gel), stable |
| MDOC-NH4 | 5.689 | 43.748 | 0.561 | 7.9 | 11.0 | 4 | swelling (opaque gel), stable |

The initial results indicate that MDOC-H+ without an additional acid component can be used for cross-linking PVA. A concentration of MDOC-H+ 8% was found to be most effective.

7. Effect of the Type of MDOC on The Mechanical Properties of the Matrix

As previously discussed above in sections III(C)(1-5), MDOC matrices were produced using both soluble and insoluble MDOC compounds, and will be discussed further in the following sections. The insoluble MDOC matrices (1 and 2) from FIG. 56 were compared to the soluble MDOC matrices (5, 6, 11, and 12). The results are shown in FIGS. 66 and 67, comparing the strengths of the nanofibrous matrices before and after sterilization, and FIGS. 68 and 69 comparing the elongation of the matrices before and after sterilization.

The initial results, though not conclusive, indicate that the type of MDOC likely does not affect the strength of the matrix, and the addition of phosphoric acid potentially adds a positive effect on the strength of the matrices. Similarly, elongation followed the same trends with both types of MDOC, with elongation being lower after sterilization. Phosphoric acid did not significantly affect the elongation characteristics.

8. Effect of the Ratio of MDOC/PVA on the Mechanical Properties

MDOC/PVA matrices containing 40% MDOC and 60% PVA were compared against one another to assess the mechanical properties of both the insoluble matrices (3, 4 and 10) and the soluble matrices (7, 8, and 9). The strengths were compared in FIGS. 70 and 71 (before and after sterilization), and elongation was compared in FIGS. 72 and 73 (before and after sterilization). The results indicated poor mechanical properties for each of the matrices at the stated ratio of MDOC and PVA.

Comparatively, a decrease in the amount of MDOC in the MDOC/PVA matrix indicated an increase in the mechanical properties of the matrix. Matrices containing 20% MDOC and 80% PVA (1 and 5) were compared to matrices containing 40% MDOC and 60% PVA (3 and 7). The results are shown in FIG. 74 and FIG. 75, which indicate an increase in mechanical properties for the matrices having a lower MDOC concentration, both for the soluble and insoluble types of MDOC.

9. Optimization of MDOC/PVA Ratio

The results indicate that there may be an optimal ration of MDOC to PVA. To accomplish this, various MDOC/PVA solutions were mixed and tested. A PVA solution (16±1.5) % m/m was purchased from Novacke chemicke zavody, Slovakia. A 16% m/m dispersion of MDOC was prepared by mixing 64.0 g of MDOC I (Batch No X01 028) with 336.6 g of deionized water using UltraTurrax T25. The MDOC and PVA solutions were mixed in ratio (weight:weight) 3:7, 4:6, 5:5, 6:4 and 7:3 using an UltraTurrax T25 mixer for 1.5 minute, which were then centrifuged (3 min., 3000 min⁻¹) to remove bubbles before electrospinning and measurements.

Approximately 25-26 g of each dispersion was placed into a spinning bath and electrospun for 15 minutes under conditions, as follows:

Equipment: NanoSpider LAB, location Ti\check{s}nov
Electrode: small smooth cylinder
Collector: wire, with spunbond
Electrode distance: 120 mm
Fabric speed: 0.3 m.min⁻¹
Electrode spin: 3.2 min⁻¹

### HV: 75.0 kV

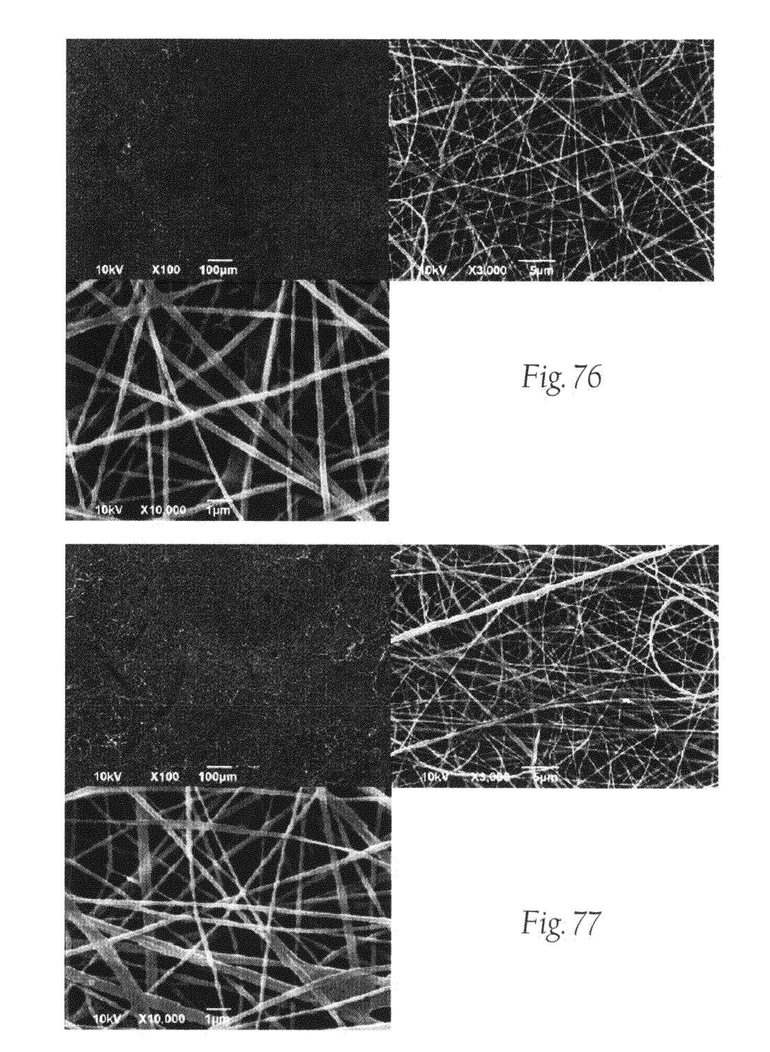

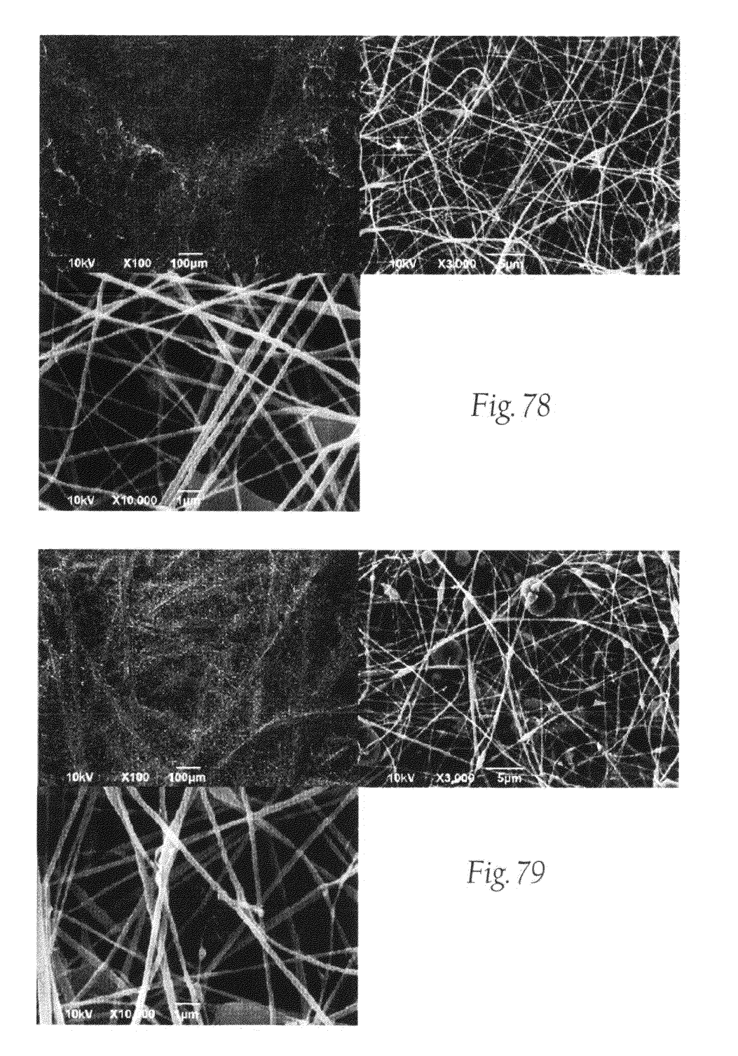

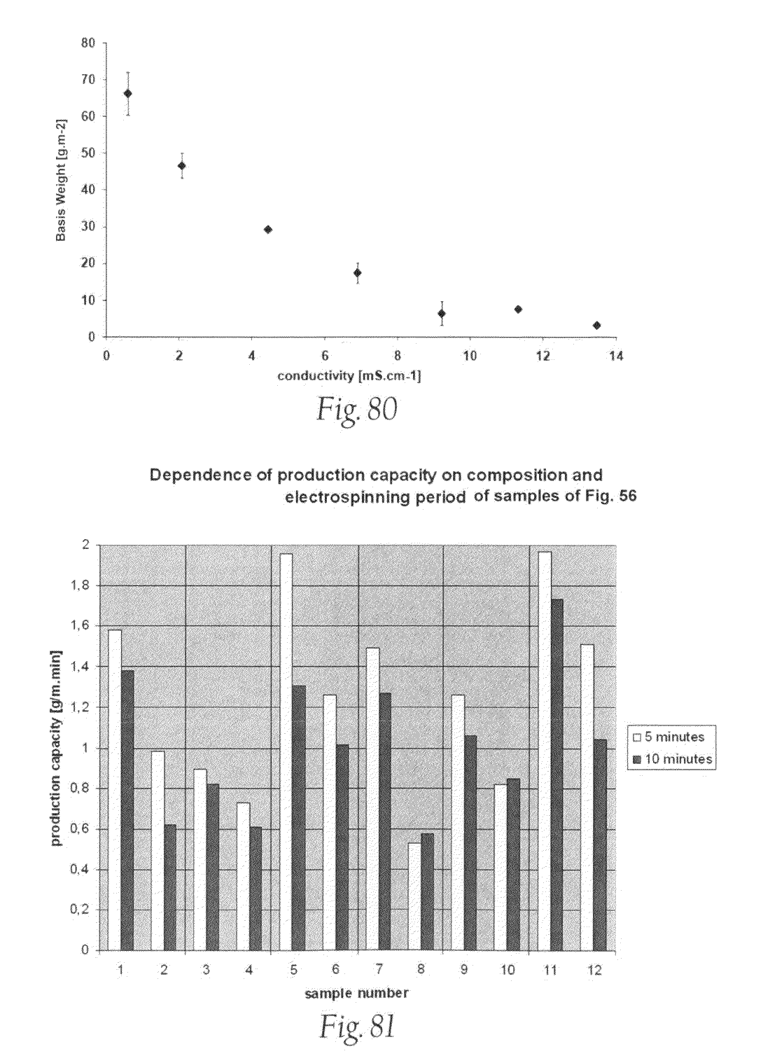

SEM analyses were completed for assessment of nanofiber quality and are shown in FIGS. 76-79. The measured values are presented in Table 25, below.

TABLE 25
Measured Values For MDOC/PVA Solutions

| | | | PVA | PVA | dry matter | dry matter | difference | temperature |
|---|---|---|---|---|---|---|---|---|
| | | | content | content | content - | content - | in dry | of dispersion |
| | PVA | | before | after | before | after | matter | before |
| MDOC | solution | conductivity | spinning | spinning | spinning | spinning | content | spinning |
| dispersion [%] | [%] | [mS · cm⁻¹] | [%] ¹ | [%] ¹ | [%] | [%] | [%] | [° C.] |
| 30.0 | 70.0 | 3.77 | 72.5 | 70.6 | 14.99 | 15.92 | 0.92 | 27.5 |
| 30.0 | 70.0 | 3.82 | 72.6 | 68.4 | 14.92 | 16.09 | 1.17 | 27.6 |
| 40.0 | 60.0 | 4.83 | 61.7 | 61.2 | 14.87 | 15.64 | 0.77 | 27.3 |
| 40.0 | 60.0 | 4.90 | 61.8 | 58.6 | 14.90 | 16.30 | 1.40 | 28.0 |
| 50.0 | 50.0 | 6.03 | 45.9 | 50.4 | 14.71 | 15.92 | 1.21 | 27.6 |
| 50.0 | 50.0 | 6.09 | 51.1 | 50.7 | 14.67 | 15.94 | 1.26 | 27.8 |
| 59.8 | 40.2 | 7.43 | 44.6 | 43.2 | 14.37 | 15.76 | 1.39 | 27.1 |
| 60.0 | 40.0 | 7.50 | 44.7 | 42.9 | 14.40 | 15.85 | 1.44 | 28.3 |
| 70.1 | 29.9 | 9.14 | 13.3 | 20.0 | 13.77 | 15.34 | 1.58 | 27.4 |
| 69.7 | 30.3 | 8.83 | 30.6 | 25.8 | 14.08 | 15.35 | 1.27 | 27.3 |

| | temperature | | viscosity of | viscosity of | | | median of |
|---|---|---|---|---|---|---|---|
| | of dispersion | difference in | dispersion | dispersion | amount | | input air |
| | after | temperature | before | after | of spun | sum of | temper- |
| MDOC | spinning | of dispersion | spinning | spinning | material | HV current | ature |
| dispersion [%] | [° C.] | [° C.] | [mPa · s] | [mPa · s] | [g] ² | [mA] ³ | [° C.] ⁴ |
| 30.0 | 21.1 | −6.4 | 544 | 669 | 7.4 | 1.84 | 24.3 |
| 30.0 | 21.6 | −6.0 | 525 | 658 | 9.8 | 2.23 | 25.8 |
| 40.0 | 22.7 | −4.6 | 430 | 772 | 5.1 | 1.62 | 24.6 |
| 40.0 | 21.7 | −6.3 | 426 | 773 | 5.5 | 1.77 | 25.8 |
| 50.0 | 21.7 | −5.9 | 747 | 1286 | 3.8 | 1.69 | 24.9 |
| 50.0 | 21.8 | −6.0 | 818 | 1303 | 4.6 | 1.93 | 25.8 |
| 59.8 | 21.1 | −6.0 | 612 | 713 | 3.5 | 1.79 | 25.2 |
| 60.0 | 21.7 | −6.6 | 446 | 695 | 3.5 | 1.81 | 25.9 |
| 70.1 | 21.9 | −5.5 | 419 | 522 | 2.6 | 1.40 | 25.4 |
| 69.7 | 21.7 | −5.6 | 355 | 405 | 3.2 | 1.65 | 25.6 |

Each of the various ratios produced acceptable results. The initial results further indicate that a 4:6 ratio of MDOC:PVA produces an electrospun material having the best quality individual nanofibers if any of the tested ratios.

10. Production Capacity

Along with testing the mechanical properties of the various matrices, the matrices described in FIG. 56 were compared at electrospinning periods of 5 and 10 minutes. The results are shown in FIG. 81. In each instance, the longer the electrospinning period resulted in a decrease in production capacity, which can be attributed to a thicker layer of material deposited on the counter electrode (see discussion with respect to FIG. 6), thereby forming a shield for the counter electrode for further deposits of the nanospun fibers.

The addition of phosphoric acid decreased production capacity, as did the addition of phosphoric acid and glyoxal. Also, insoluble MDOC has lower production capacity than soluble MDOC, and higher MDOC content decreases production capacity.

11. The Effect of Solution Conductivity on PVA Electrospinning

Solution conductivity is reported an important parameter for electrospinning process. The following section attempts to correlate the solution conductivity of a PVA solution with the resultant product. Seven PVA solutions with constant PVA concentration and different concentration of sodium chloride (NaCl) were prepared and electrospun.

A PVA solution (16±1.5) % m/m was purchased from Novacke chemicke zavody, Slovakia. Stock NaCl solution (2 mol/l) was prepared by weighing 23.4 g NaCl, dissolving and diluting to 200 ml by deionized water. This solution was further diluted to 1.64, 1.28, 0.92, 0.56 and 0.2 mol/l. 10.0 g of NaCl solution (or water) was then mixed with 90.0 g of PVA solution (16±1.5) % resulting in approx 14% PVA solution with different conductivity ranging from 0.58 (PVA solution with water) to 13.5 mS/cm. The various solutions are shown in Table 26. The solutions were mixed using an UltraTurrax mixer for 1.5 minute and centrifuged (3 min, 3000 RPM) to remove bubbles before electrospinning and measuring. The various solutions are shown in Table 26.

Approximately 25-26 g of PVA/NaCl solution were placed into a spinning bath and electrospun for 15 minutes under the conditions described, below.

Equipment: NanoSpider LAB, location Tignov
Electrode: small smooth cylinder
Collector: wire with spunbond fixed
Electrode distance: 130 mm
Fabric speed:
Electrode spin: 3.2 min⁻¹

### HV: 80.0 kV

The results are shown in Table 26. A comparison of the conductivity to the area weight is shown in FIG. 80.

TABLE 26
Solution Conductivity for PVA Solutions

| conductivity [mS · cm⁻¹] | 13.47 | 11.33 | 9.22 | 6.89 | 4.44 | 2.07 | 0.58 |
|---|---|---|---|---|---|---|---|
| dry matter content - before | 14.71 | 14.53 | 14.31 | 14.11 | 13.93 | 13.35 | 13.44 |
| spinning [%] | | | | | | | |
| dry matter content - after | 15.22 | 15.09 | 14.82 | 14.44 | 13.98 | 14.15 | 13.28 |
| spinning [%] | | | | | | | |
| viscosity before spinning [mPa · s] | 884 | 858 | 872 | 869 | 846 | 821 | 820 |
| temperature of dispersion before | 27.3 | 28.0 | 27.7 | 28.1 | 27.8 | 28.3 | 28.0 |
| spinning [° C.] | | | | | | | |
| temperature of dispersion after | 21.1 | 21.4 | 21.9 | 21.8 | 22.1 | 22.3 | 22.7 |
| spinning [° C.] | | | | | | | |
| difference in temperature of | −6.2 | −6.6 | −5.8 | −6.3 | −5.7 | −6.0 | −5.3 |
| dispersion [° C.] | | | | | | | |
| median of input air temperature | 23.8 | 23.8 | 23.8 | 23.8 | 24.1 | 24.3 | 24.5 |
| [° C.] | | | | | | | |
| sum of HV current [mA] | 1.16 | 1.65 | 1.42 | 1.83 | 1.77 | 1.73 | 1.05 |
| amount of spun material [g] | 2.1 | 32 | 3.0 | 5.8 | 8.7 | 14.1 | 18.8 |
| area weight [g · m⁻²] | 3.2 | 7.6 | 6.4 | 17.4 | 29.3 | 46.6 | 66.2 |
| dry matter content - nanofiber | N/A | 97.5 | 95.5 | 94.2 | 94.4 | 94.7 | 94.6 |
| [%] | | | | | | | |
| Fiber diameter [nm]¹⁾ | 170 | 150 | 140 | 130 | 110 | 120 | 140 |

The results indicate that viscosity of the PVA solutions is slightly influenced by NaCl. It is increased by about 8% for PVA/NaCl solution of the highest NaCl content.

The results also show that the amount of spun material and area weight decrease with increasing conductivity of solution, as shown in FIG. 80.

Visual analysis of the spun materials indicated that the quality (lower content of thick fibers and ribbon-like defects) of a nanofiber layer is better for a PVA solution of lower conductivity. However, the initial results indicate that it may be possible to optimize the quality of an electrospun material based on an individual basis.

12. MDOC/PVA/PEO Matrix Further Comprising Chlorhexidine

The following section discusses electrospinning an MDOC/PVA/PEO matrix, which further includes chlorhexidine. Specifically, chlorhexidine hydrochloride solution was added to the matrix, which was calculated on the dry matter content of solution MDOC/PVA/PEO. The dry matter content of MDOC/PVA/PEO solution was 14.16% (with a ratio 40/57/3 wt %, respectively). The tested solutions had concentrations of chlorhexidine dihydrochloride as 1, 2.5, 5, 10, and 20 wt % related to MDOC/PVA/PEO dry matter content.

The electrospinning solution was formed by mixing chlorhexidine dihydrochloride in water. MDOC was added and the formed mixture was agitated using an UltraTurrax T25 for approximately 2 minutes. PVA was then added and the mixture was agitated using an RzR 2041 stirrer for approximately 10 minutes and then was allowed to stand for approximately 12 hours. Finally, a PEO water solution was added and the formed mixture was blended using the RzR 2041 stirrer for approximately 60 minutes.

The electrospinning process was carried out so that each of the tested samples had a relatively similar basis weight. Thus, the electrode distance was changed during the spinning process too keep uniformity with the spun layers. Changing of the electrode distance also allowed for a constant current value for all samples during the process and thereby provide similar basis weight of all prepared samples. It seems that process efficiency decreases with increasing chlorhexidine dihydrochloride concentration. All data of spinning process are listed in Table 27, below.

TABLE 27
Data of spinning process for MDOC/PVA/PEO
samples with chlorhexidine

| | chlorhexidine | | tem- | | |
|---|---|---|---|---|---|
| | amount | Electrode | pera- | | Basis |
| | [wt % to dry | distance | ture | RH | weight |
| Sample | matter] | [mm] | [° C.] | [%] | [g/m²] |
| D090930MC01 | 1 | 160 | 24.2 | 28.3 | 13.8 |
| D090930MC02 | 2.5 | 160 | 23.1 | 30.3 | 14.2 |
| D090930MC04 | 5 | 155 | 23.1 | 30.8 | 14.3 |
| D090930MC05 | 10 | 155 | 22.6 | 29.6 | 14.1 |
| D090930MC03 | 20 | 150 | 24.1 | 29.2 | 11.2 |

| | | | |
|---|---|---|---|
| Substrate | 13 | HV [kV] | 70-82 |
| speed [cm · min⁻¹] | | | |
| RPM of | 2.2 | Number of passes | 5 |
| spinning | | | |
| electrode | | | |

The matrix comprising MDOC/PVA/PEO with chlorhexidine hydrochloride was successfully spun, in concentrations from 1 to 20 w % chlorhexidine to dry matter content). The process efficiency appeared to with an increased concentration of chlorhexidine dihydrochloride, which was believed to correlate to the insolubility of chlorhexidine material in water. Initial results, while not conclusive, indicate that chlorhexidine materials can be incorporated into an electrospun material, which may provide beneficial results.

D. Gelatin Materials

The following examples discuss the potential of electrospinning gelatin based materials, including gels and other similar materials.

1. Combinations of Gelatin with Various Polymers

Porcine gelatin materials were combined with various non-animal source polymers to determine the spinability of the various combinations. Tested polymers included CMC, PEO, PVA, MDOC and MDOC H⁺ form.

Gelatin was purchased from Gelita AG, Eberbach, Germany. High molecular weight Gelita gelatin was used for the initial tests, since this type of gelatin can be readily electrospun alone from acetic acid solution, or as a mixture with polymers mentioned above. This gelatin solution has relative low viscosity, so increasing the viscosity by the addition of polymers like CMC did not cause any problem in electrospinning. A 9% gelatin materials in 80% w/w acetic acid solution was heated to 50° C. for approximately 2.5 hours. The solution was then cooled to room temperature, and the desired polymer was added as an aqueous solution to the gelatin solution after cooling down of gelatin solution to laboratory temperature. Compositions of all solutions are mentioned below.

All mixtures were electrospun using the same electrode system (cylinder-cylinder) and NS-Lab machine. For each mixture, the distance between electrodes was changed—current was higher than maximum (1.8 mA) for mixtures gelatin/PEO. Setting of the NS-Lab is, mentioned below in Table 28.

TABLE 28
Used setting on NS-Lab machine

| | Number | Substrate | | Electrode | | | spinning |
|---|---|---|---|---|---|---|---|
| | of | speed | HV | distance | | RH | electrode |
| Sample | passes | [Hz] | [kV] | [mm] | T[° C.] | [%] | [Hz] |
| A090925GE01 | 2 | 5 | +55 | 240 | 20.1 | 30.3 | 6 |
| A090925GE02 | 1 | 5 | +74.5 | 142 | 20.1 | 30.3 | 6 |
| A090925GE03 | 1 | 5 | +81.9 | 173 | 20.3 | 30.5 | 10.4 |
| A090925GE04 | 1 | 5 | +81.9 | 150 | 20.3 | 30.5 | 10.4 |
| A090925GE05 | 1 | 5 | +81.9 | 175 | 20.3 | 30.5 | 10.4 |

The Solutions and sample data are shown below in Tables 29-34.

TABLE 29
Solution code: GE090925V01 -
Initial Gelatin Composition

| | | | Expected Dry |
|---|---|---|---|
| Raw Material | Weight (g) | Weight % | Matter (g) |
| Gelatin- | 180.00 | 9.00 | 180.00 |
| GELITA | | | |
| Demi water | 349.40 | 17.47 | N/A |
| Acetic Acid | 1470.60 | 73.53 | N/A |
| Total Weight | 2000.00 | 100.00 | 180.00 |

TABLE 30
Solution code: GE090925V02 - gelatin/PEO 8:2

| | Weight | | Expected dry |
|---|---|---|---|
| Raw material: | (g) | Weight % | matter (g) |
| gelatin - GELITA | 27.00 | 5.60 | 27.00 |
| PEO | 6.75 | 1.40 | 6.75 |
| demi water | 227.97 | 47.27 | N/A |
| acetic acid | 220.59 | 45.73 | N/A |
| Total weight | 482.31 | 100.00 | 33.75 |

TABLE 31
Solution code: GE090925V03 - gelatine/PVA 6:4

| | Weight | | Expected dry |
|---|---|---|---|
| Raw material: | (g) | Weight % | matter (g) |
| gelatin - GELITA | 26.98 | 6.67 | 26.98 |
| PVA | 16.76 | 4.14 | 16.76 |
| demi water | 140.35 | 34.70 | N/A |
| acetic acid | 220.41 | 54.49 | N/A |
| Total weight | 404.50 | 100.00 | 43.74 |

TABLE 32
Solution code: GE090925V04 - gelatine/CMC 7:3

| | Weight | | Expected dry |
|---|---|---|---|
| Raw material: | (g) | Weight % | matter (g) |
| Gelatin- GELITA | 26.96 | 8.32 | 26.96 |
| CMC | 11.57 | 3.57 | 11.57 |
| demi water | 65.41 | 20.17 | N/A |
| acetic acid | 220.30 | 67.94 | N/A |
| Total weight | 324.24 | 100.00 | 38.53 |

TABLE 33
Solution code: GE090925V05 - gelatine/MDOC 7:3

| | Weight | | Expected dry |
|---|---|---|---|
| Raw material: | (g) | Weight % | matter (g) |
| gelatin GELITA | 26.98 | 8.30 | 26.98 |
| MDOC | 11.57 | 3.56 | 11.57 |
| demi water | 65.88 | 20.28 | N/A |
| acetic acid | 220.45 | 67.86 | N/A |
| Total weight | 324.88 | 100.00 | 38.55 |

TABLE 34
Solution code: GE090925V06 - gelatine/MDOC H⁺ 7:3

| | Weight | | Expected dry |
|---|---|---|---|
| Raw material: | (g) | Weight % | matter (g) |
| gelatin - GELITA | 27.00 | 8.34 | 27.00 |
| MDOC H⁺ | 11.57 | 3.58 | 11.57 |
| demi water | 64.51 | 19.93 | N/A |
| acetic acid | 220.59 | 68.15 | N/A |
| Total weight | 323.67 | 100.00 | 38.57 |

Each of the gelatin combinations was capable of being electrospun. Generally, the presence of PEO in gelatin solution significantly increased the electric current during the spinning, and the formed nanolayer of material was visibly wet, which could be a potential combination for use with certain gelatin having lower molecular weight than the GELITA gelatin used in the present example.

Further initial indications seem to show that high molecular porcine gelatin (contrary to low molecular one) can be electrospun directly from CMC and/or MDOC mixtures without any other supporting fiber-forming polymers such as PVA or PEO.

2. Optimization of Gelatin Solution with PEO

As indicated by the previous section, spinning of gelatin materials in combination with other compounds was possible, with PEO being one such compound. The following example further tests an optimal gelatin/PEO combination for electrospinning, using a water-acetic acid solvent system to carry out the testing. The system is applicable at room temperature and does not require a co-polymer.

Several combinations of gelatin/acetic acid concentration and suitable combinations of electrode-collector system for electrospinning of gelatin were tested.

Initial analysis of the effect of gelatin and/or acetic acid concentration on microscopic quality of nanofiber layer indicated that the most suitable solutions was a solution having 9% gelatin dissolved in 80% acetic acid. The effects on the electrode system was further investigated. Three electrodes, cylinder collector and three distance values were set and electrospinning process was performed under HV which was found suitable and sufficient for continuous spinning process.

The gelatin solution was formed by mixing GELITA gelatin: Pigskin gelatin, 300 Bloom, Pharm. grade, purchased from Gelita, with water and left standing for 1 hour. The swollen gelatin was further mixed with acetic acid: 99%, p.a., purchased from Penta, CZ. Suitable amount of gelatin was weighed, using magnetic stirrer for at least 40 minutes for complete dissolution. The time required for complete dissolution depends on the amount of gelatin and concentration of acetic acid.

As an example, 54.0 g of gelatin was weighed, mixed with 109.2 g of water and left standing for 1 hour. Then 437.7 g of acetic acid was added to swollen gelatin and mixed using magnetic stirrer for 2 hours for complete dissolution (a larger amount of gelatin requires a longer time for complete dissolution). Final concentration of acetic acid in diluent is 80% w/w and final concentration of gelatin in solution is 9% w/w. Amount and final concentration of components can be found in Table 35.

Approximately 25 g of gelatin/acetic acid solution were placed into spinning bath and electrospun for 10 minutes under conditions described below. Small amount of fresh polymer blend was added to the bath after each spinning to maintain total weight of solution at 25 g.

Equipment: NanoSpider LAB, location Ti\check{s}nov
Electrode: small cylinder
Collector: needles with spunbond fixed
Electrode distance: see Table 1
Fabric speed: 0, static spunbond
Electrode spin: 3.2 min⁻¹

### HV: see Table 1

TABLE 35
Combinations of gelatin - acetic acid concentration and other parameters of electrospinning.

| | | | Acetic acid in | GELITA in | | | |
|---|---|---|---|---|---|---|---|
| GELITA | Water | Acetic acid | solvent | solution | Viscosity | Conductivity | HV |
| [g] | [g] | [g] | [g] | [g] | [mPa · s] | [mS · sm⁻¹] | [kV] |
| 3.75 | 23.12 | 23.10 | 50 | 7.5 | 67 | 1197 | 82.0¹⁾ |
| | 14.00 | 32.34 | 70 | | 93 | 768 | 82.0¹⁾ |
| | 4.68 | 41.58 | 90 | | 100 | 340 | 70.5 |
| 5.01 | 22.48 | 22.47 | 50 | 10.0 | 121 | 1378 | 82.0 |
| | 13.67 | 31.50 | 70 | | 215 | 1054 | 62.5 |
| | 4.73 | 40.53 | 90 | | 209 | 440 | 65.0 |
| 6.25 | 22.06 | 21.84 | 50 | 12.5 | 214 | 1533 | 70.0 |
| | 13.13 | 30.66 | 70 | | 322 | 1014 | 60.0 |
| | 4.31 | 39.38 | 90 | | 442 | 480 | 60.0 |

| | | Median of | Median of | Amount | Average | Fiber | |
|---|---|---|---|---|---|---|---|
| | Electrode | input air | input air | of spun | fiber | diameter | Overall |
| GELITA | distance | temperature | RH | material | diameter | range | microscopic |
| [g] | [mm] | [° C.] | [%] | [g] | [nm] | [nm] | quality |
| 3.75 | 150 | 23.5 | 49.9 | 4.5 | 80 | 60-100 | 444 |
| | 150 | 23.6 | 51.5 | 5.3 | 110 | 90-140 | 223 |
| | 160 | 24.2 | 50.9 | ²⁾ | 210 | 170-310 | 212 |
| 5.01 | 160 | 23.9 | 51.3 | 4.1 | 100 | 70-150 | 422 |
| | 160 | 24.5 | 51.6 | 4.2 | 150 | 100-200 | 212 |
| | 170 | 24.7 | 50.9 | 8.1 | 460 | 230-1770 | 123 |
| 6.25 | 180 | 24.7 | 50.6 | 4.3 | 160 | 110-220 | 211 |
| | 180 | 24.8 | 50.4 | 4.3 | 240 | 220-260 | 212 |
| | 190 | 24.9 | 50.2 | 10.8 | 930 | 400-3200 | 234 |

Approximately 450 g of a gelatin/acetic acid solution were placed into spinning bath and electrospun for 15 minutes under conditions described below. Small amount of fresh polymer blend was added to the bath after each spinning cycle to maintain total weight of solution at 450 g.

Equipment: NanoSpider LAB, location Tinov

### Electrode: see Table 2

Collector: cylinder
Electrode distance: see Table 2
Fabric speed: 0, static spunbond
Electrode spin: 3.2 min⁻¹

TABLE 36
Combinations of electrode - cylinder collector and other parameters of
electrospinning. 9% gelatin in 80% acetic acid, viscosity 160 mPa · s.

| | | | Median of | Median of | Amount | Average | Fiber | |
|---|---|---|---|---|---|---|---|---|
| | | Electrode | input air | input air | of spun | fiber | diameter | Overall |
| | HV | distance | temperature | RH | material | diameter | range | microscopic |
| Electrode | [kV] | [mm] | [° C.] | [%] | [g] | [nm] | [nm] | quality |
| cylinder | 75 | 150 | 23.6 | 54.1 | 33.3 | 144 | 85-295 | 322 |
| | 80 | 175 | 25.2 | 51.2 | 25.4 | 147 | 80-250 | 223 |
| | 82 | 200 | 25.5 | 50.4 | 18.4 | 134 | 75-280 | 433 |
| 6-wire | 45 | 150 | 25.7 | 49.6 | 13.4 | 152 | 85-280 | 422 |
| | 50 | 175 | 25.8 | 49.5 | 13.4 | 153 | 70-280 | 323 |
| | 55 | 200 | 26.0 | 48.9 | 14.1 | 168 | 100-400 | 323 |
| lamella | 65 | 150 | 26.0 | 48.4 | 22.9 | 143 | 80-290 | 433 |
| | 70 | 175 | 26.0 | 48.4 | 24.3 | 153 | 90-330 | 333 |
| | 77.5 | 200 | 26.4 | 47.2 | 23.9 | 182 | 95-430 | 233 |

The results indicated that the use of gelatins in electrospinning processes has potential. Generally, it was visually observed that the lower concentrations, resulted in more “macro defects” in the electrospun materials, while higher concentration of gelatin or acetic acid, however, results in thicker fibers and formation of ribbon-like fibers. As such, it was initially assessed that using 9% gelatin in 80% acetic acid would be a compromise between low number of macro defects (droplets), low number of ribbon-like thick fibers and acceptable fiber diameter. However, the results indicate that other gelatin solutions are spinnable and may have potential uses, depending on the particular requirements or need for a particular use.

E. Electrospun Hyaluronic Solutions

Electrospinning of hyaluronic acid was conducted, as explained below. The hyaluronic acid was supplied from Dermachem Cosmetic Ingredients, LLC, located in Tampa, Fla., and has a high molecular weight (value on the label: 1070 kDa).

An aqueous 5 wt % solution of hyaluronic acid was prepared. As the solution was very viscous, it was diluted to a concentration of 1.71 wt % hyaluronic acid in water. The solution was further blended with 3 wt % PEO in water for further diluting. Thus, the solution was mixed again with a Turrax mixed (2 minutes, 6000-10000 rpm) and electrospinning was reattempted. We used smooth small cylinder and thorn, but process did not start at all.

A 5 wt % solution in 80 wt % acetic acid was prepared. When acetic acid to hyaluronic acid was added to the solution, a precipitation was observed. The precipitation disappeared when other water was added. We used such volumes of acetic acid and water to obtain a concentration around 1.7 wt % of hyaluronic acid in 58.66 wt % acetic acid. We added calcium chloride to the solution in ratio 56:44 (hyaluronic acid: calcium chloride w:w) for decreasing of its viscosity because inorganic salt generally decreases viscosity of HA water solution. As no change in viscosity was observed after the stirring, the solution was heated to 45° C. for 3 hours. After cooling of this solution the viscosity stayed still visually high and solution was not spinnable. The solution was stored without any stirring for one week. The 7 day old solution was spinnable together with 3 wt % PEO in ratio 60:40 HA:PEO in dry matter. The solution was spun both using a thorn and a smooth cylinder. Compositions of all solutions spun are mentioned below in Tables 41-44

TABLE 41

| | Weight | | Expected dry matter |
|---|---|---|---|
| Raw material: | (g) | Weight % | (g) |
| Solution 1 - | | | |
| HA090904V01 | | | |
| hyaluronic | 0.800 | 1.71 | 0.800 |
| acid | | | |
| demi water | 46.070 | 98.29 | N/A |
| Total weight | 46.870 | 100.00 | 0.800 |

TABLE 42

| | Weight | | Expected dry matter |
|---|---|---|---|
| Raw material: | (g) | Weight % | (g) |
| Solution 2 - | | | |
| HA090904V02 | | | |
| hyaluronic | 0.1026 | 1.08 | 1.08 |
| acid | | | |
| demi water | 9.2924 | 97.81 | N/A |
| PEO | 0.1050 | 1.11 | 1.11 |
| Total weight | 9.5000 | 100.00 | 2.19 |

TABLE 43

| | Weight | | Expected dry matter |
|---|---|---|---|
| Raw material: | (g) | Weight % | (g) |
| Solution 3 - | | | |
| HA090908V01 | | | |
| hyaluronic | 1.5046 | 1.66 | 1.5046 |
| acid | | | |
| demi water | 36.2502 | 40.12 | N/A |
| Acetic acid | 51.4335 | 56.92 | N/A |
| (99%) | | | |
| Calcium | 1.1750 | 1.30 | 1.1750 |
| chloride | | | |
| Total weight | 90.3633 | 100.00 | 2.6796 |

TABLE 44

| | Weight | | Expected dry matter |
|---|---|---|---|
| Raw material: | (g) | Weight % | (g) |
| Solution 4 - | | | |
| HA090914V01 | | | |
| hyaluronic acid | 1.5046 | 1.22 | 1.5046 |
| demi water | 68.6967 | 55.48 | N/A |
| Acetic acid (99%) | 51.4335 | 41.54 | N/A |
| Calcium chloride | 1.1750 | 0.95 | 1.1750 |
| PEO | 1.0035 | 0.81 | 1.0035 |
| Total weight | 123.8133 | 100.00 | 3.6831 |

The results indicated that the inorganic salt addition had an effect on the process. While 1% HA water solution had a viscosity around 2900 mPa·s, the same solution with NaCl addition (the concentration of NaCl was approximately 0.07 mol/l) had a viscosity of only 980 mPa·s.

Effect of various pH and MDOC addition on viscosity of 1% of HA water solution was also studied. The following-three mixtures were prepared:

1.20 g of 1% water solution was mixed with 0.5 ml of HNO₃ (pH<1)

2. 20 g of 1% water solution+0.5 ml of 30% w/w NaOH solution (pH=12)

3. 20 g of 1% water solution+200 mg of MDOC
Viscosity of the 2^{nd} solution dramatically decreased to approximately 2 mPa·s immediately after the alkali addition. The addition of ammonia instead of NaOH was also tested to determine its effect on viscosity. The viscosity of the hyaluronic solution after the ammonia addition did not drop as much as with strong base, but was visually assessed as slightly lower compared to original solution. Viscosity of the solution with nitric acid was more or less comparable with the original solution and presence of MDOC decreased the viscosity to ½. All three solution were then put to a climatic chamber at 40° C. and RH=80% for 5 days. Viscosity of all three solutions significantly decreased during the storage:
solution 1: η²⁵=1.3 mPa·sec
solution 2: η²⁵=1.1 mPa·sec (solution discolored to a yellow hue)
solution 3: η²⁵=97 mPa·sec (grew mold during the storage)
(viscosity of original 1% water solution was approximately 2900 mPa·s)
Solution No 1 and 2 were mixed with 3% PEO in the ratio 60/40 vol/vol. Both solutions willingly spun, but no final nanolayer was obtained due to low concentration of HA in spun solution(s) (only dirty base material was obtained). The results indicate that it may be necessary to increase concentration of hydroaluronic acid in water solution prior to hydrolysis.
Solution 1 and 2 were sampled for GPC analysis and will be compared with a freshly prepared solution.

The 7 day old solution of hyaluronic acid in acetic acid with addition of CaCl₂ and PEO (HA090914V01) was successfully spun (i.e. it was obtained a tangible nanolayer. Smooth cylinder as spinning electrode and cylinder as a collector were used. Distance between electrodes was 14-16 cm, rotation of spinning electrode 12-15 Hz and applied high voltage 81.9 kV (with current ca 0.4 mA). This experiment has been done at 25% relative humidity and 21° C. This solution has a tendency to create a generally 3-D, i.e. fluffy, structure.

As such, the result 7 days old solution of HA in acetic acid with CaCl₂ addition was spinnable with PEO 3% w/w in ratio 60:40 (this ratio was used for sodium salt of hyaluronic acid in Nanopeutics). Tested type of hyaluronic acid having a molecular weight around 1070 kDa was not spinnable either from water or acetic acid solutions (freshly prepared) due to their high viscosities. The initial results, while not conclusive, indicate that there is a possibility to optimize the ratio HA:PEO (to increase HA content).

F. Electrospun CMC/PVA/PEO Solution with Silver Nanoparticles

The following example was carried out to provide an electrospun CMC/PVA/PEO nanomaterial, that further incorporated silver nanoparticles at concentrations of 100 and 300 ppm. The example attempted to electrospin the materials so that they would form a three-dimensional layer of nanomaterial, or a “fluffy” layer of nanomaterial.

### Preparation Cmc/Pva/Peo Solution

A 6 wt % CMC/PVA/PEO solution was prepared—in ratio 2:1:1 to dry matter content. Each of the CMC—6 wt % solution and the PEO—6 wt % solutions was prepared separately and subsequently mixed together with PVA and water. SmartSilver® silver nanoparticles, supplied by NanoHorizons, Inc., located in State College, Pa., were added to the PEO solution before the final solution blending. Phosphoric acid (8 wt % to dry matter content) was finally added to the solution. Three solutions were prepared (100 ppm silver, 300 ppm silver, and no silver), and each of the solutions was post-treated by heating (due to the samples solubility in water). A comparison of the electrospun materials is shown below in Table 45

TABLE 45
Comparison of Electrospun CMC/PVA/PEO
Materials Containing Various Amounts of Silver
Particles

| | | Addition of | Addition of |
|---|---|---|---|
| | No Added | 100 ppm | 300 ppm |
| | Silver | silver | silver |
| Basis Weight | 40-43 g/m²] | 34-38 g/m²] | 37-40 g/m²] |
| Absorption | 24.3 g/g | 28.5 g/g | 28.88 g/g |
| Capacity (Acₜ) | | | |
| Fiber Diameter | 105 ± 48 nm | 165 ± 70 nm | 270 ± 130 |
| Electrospinning | 31.13 g · m⁻¹/h | 27.00 g · m⁻¹/h | 28.88 g · m⁻¹/h |
| Head Output | | | |
| HV (kV) | +65/−33 | +65/−31.5 | +65/−32 |
| Temperature (° C.) | 25.3 | 23.5 | 25.0 |
| RH (%) | 32.3 | 35.1 | 30.8 |
| Substrate Speed | 2.5 cm/min | 2.5 cm/min | 2.5 cm/min |
| Electrode | 130 mm | 137 mm | 135 mm |
| distance | | | |
| RPM of spinning | 3 | 2.5 | 3 |
| electrode | | | |

The results indicated that the addition of the silver particles to the CMC/PVA/PEO material was negligible on the spinability of the material. That is, each of the samples containing silver particles was able to be spun and produce three-dimension or “fluffy” substrates of nanomaterial. The sample with 100 ppm of silver nanoparticles was nearly identical to the sample without silver, and the sample with 300 ppm was less three-dimensional and more compact than the sample without silver particles. As such, the results indicate that there is the potential to add silver particles to layers of material, if desired.

G. Comparison of Hemostatic Qualities on Various Electrospun Materials

As discussed above, a wide range of materials can be electrospun to form matrices, with the matrices having varying qualities. One of the more important qualities for the matrices is hemostasis, which is, to some extent, a requisite for most wound dressing assemblies. Two tests were carried out to compare hemostatic properties potential wound dressing assemblies. The first test compared various electrospun nanomaterial matrices against one another, while the second test compared electrospun wound dressing assemblies compared to non-electrospun chitosan wound dressing assemblies.

1. Comparison of Hemostatic Qualities of Electrospun Nanomaterials

Five different wound dressing assemblies samples and two of these five wound dressing assemblies in combination with one another were examined for their haemostatic ability. Blood clotting capacity was determined by evaluating activation of the intrinsic pathway of the blood coagulation cascade, this assay was performed by measuring contact activation enzymes (Factor XII and kallikrein) activity. Platelet aggregation assays were employed to determine if wound pads activated platelets in platelet rich plasma (PRP). Also whole blood clotting time assays were used to measure the complete blood clotting capacity of the wound pad samples.

### Methods

Coleman Whole blood clotting test

Blood was drawn from healthy, non-aspirin-using volunteers using butterfly needles. Subsequently, 0.9 ml aliquots of the non-anti-coagulated blood was rapidly but gently, distributed into reagent-loaded (1.4×1.4 cm wound pad samples presoaked in deionized water) polystyrene tubes. There were three tubes for each determination. The tubes were tilted in a sequential manner and clot times were determined by the obvious formation of clots. The first tube was tilted at regular intervals until clotting was observed. Then the second tube was tilted until clotting was seen. This was repeated for the third tube and the clotting time of this tube was recorded as the clotting time of the hemostat.

Clot times were expressed as the average of the three independent experiments and time zero was taken as the time the first tube was tilted.

### Calculations

To deem whether a sample is haemostatic or not:

$$X = \pm \frac{( positive control - negative control )}{negative control} + 2$$

$$X = \pm \frac{( 21.7 - 100 )}{2} + 21.7$$

$$X = 60.85$$

For whole blood clotting times, values less than 60.85% are haemostatic and values equal to or less than the positive control are very haemostatic.

### Results

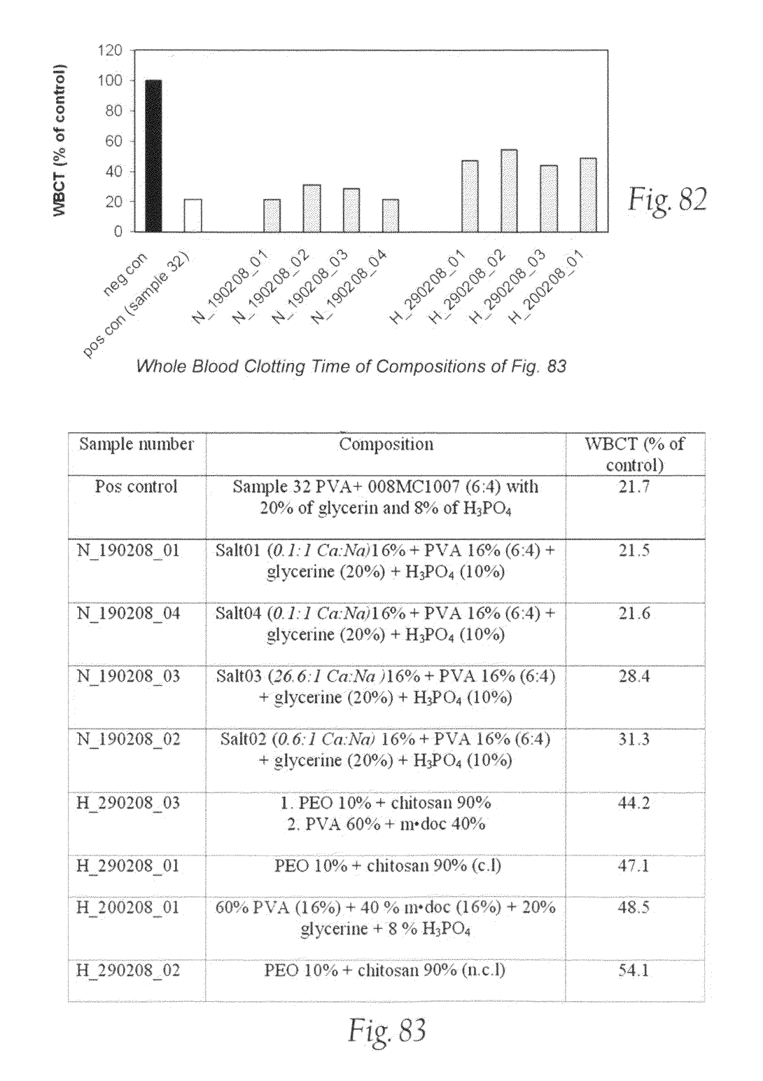

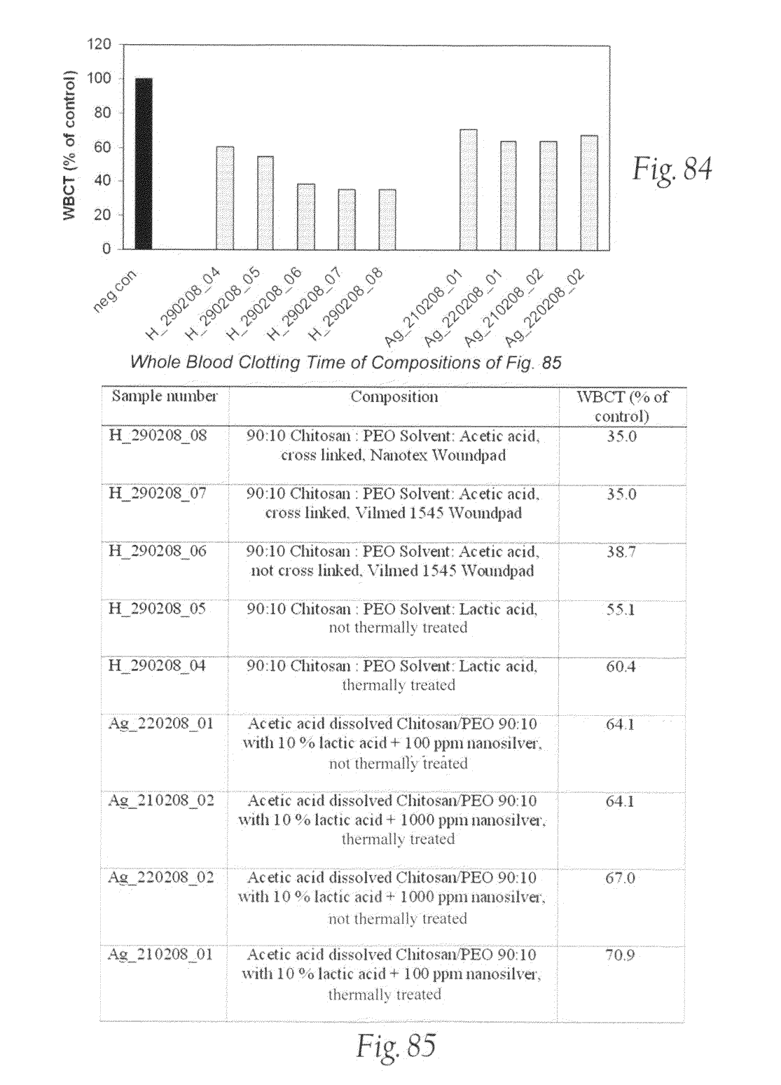

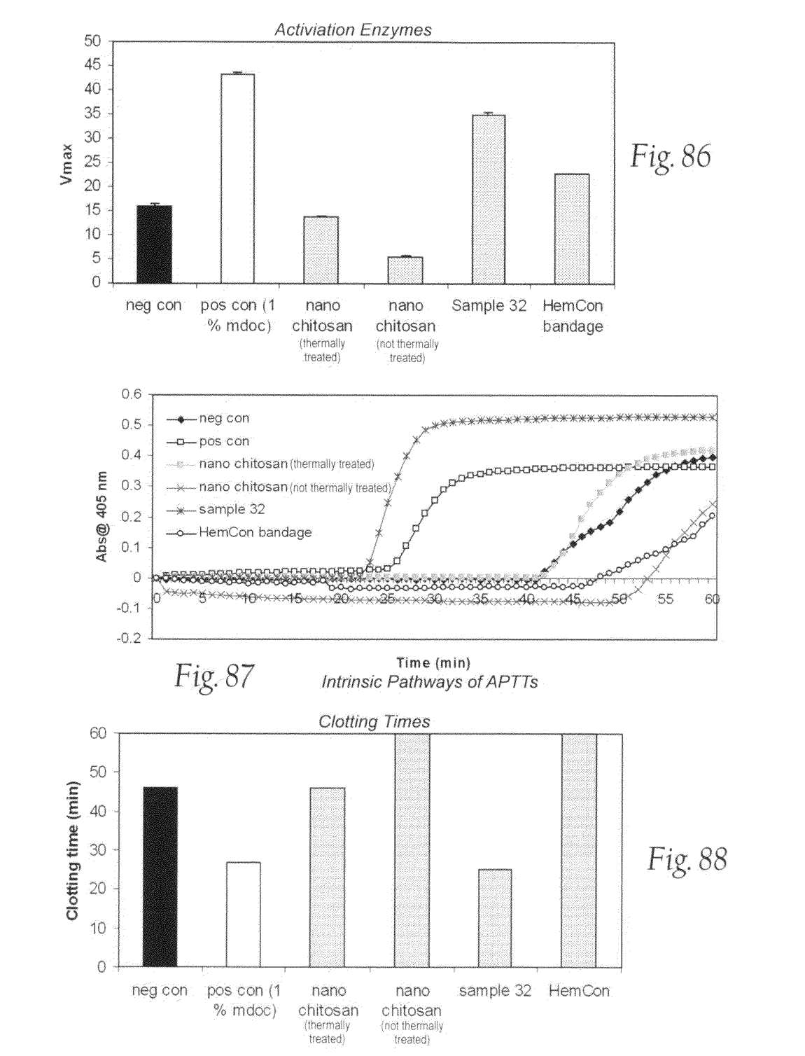

The results are shown in FIGS. 82, 83, 84, and 85. The results are based on the percentage of clotting time required for each of the samples, with a lower percentage indicating a better resultant hemostatic dressing, i.e. a faster clotting time for the dressing. The majority of the samples tested were haemostatic except for the samples containing silver.

2. Comparison of Electrospun Wound Dressing Assemblies to Other Types of Wound Dressing Assemblies

The hemostatic properties of wound dressing assemblies produced according to the present invention were compared to the hemostatic properties of prior art wound dressing assemblies. Particularly, chitosan-based wound dressing assemblies produced and manufactured by HemCon, Inc. were tested against electrospun wound dressings of the present invention that contained nanomaterial chitosan and nanomaterial MDOC. All samples were examined for their haemostatic ability. Blood clotting capacity was determined by utilizing a range of clotting assays. This gives a detailed account of the modes of blood clotting action of the samples tested. Enzyme activity assays were carried out to determine activation of the contact phase of the intrinsic pathway of the blood coagulation cascade, this assay was performed by measuring contact activation enzymes (Factor XII and kallikrein) activity. Also assays to measure the intrinsic and extrinsic pathways of blood coagulation were utilised. Platelet aggregation assays were employed to determine if samples activated platelets in platelet rich plasma (PRP). Also whole blood clotting time assays were utilised to measure the entire blood clotting capacity of samples.

### Nano Chitosan Sample

Nano chitosan samples were prepared at a density of g/m²s and contain 10% PEO and 90% chitosan, as previously discussed above. A non-crosslinked sample and a crosslinked sample (130° C., 10 min in drying chamber) were tested.

### Sample 32

Sample 32 is an MDOC nanomaterial dressing assembly containing PVA+008MC1007 (6:4) with 20% of glycerin and 8% of H₃PO₄ related to the dry mass of the basic blend. 008MC1007 is the MDOC component. As discussed above, MDOC is an oxidized cellulose composing of calcium and sodium salts. Nanofibers are impregnated with MDOC and are arranged into nanopads by spinning fibers to a nano-scale. It forms a very thin layer which does not dissolve in water.

### HemCon Bandage

The HemCon wound dressing assembly is a HemCon® Bandage commercially sold and distributed by HemCon, Inc. of Portland, Oreg.

### Methods

### Enzyme Activity Assay

5 mg samples were weighed and added to 1 ml of 100 mM HEPES buffer in an eppendorf tube. Tubes are rotated for 30 minutes. Equal volumes of sample solution and human plasma were mixed and allowed to stand for 10 minutes. 40 ml of this mixture was added to 40 ml of 0.8 mM S-2302 (see below for details). The absorbance change at 405 nm was kinetically measured. This enabled measurement of the activation of the clotting system. HEPES buffer acted as a negative control and 1% m·doc powder made up in HEPES buffer acted as the positive control.

### Measurement Principle

The Factor XII and kallikrein enzymes (clotting factors) become activated in the presence of m·doc·. The plasma kallikrein-like activity catalyses the splitting of p-nitroaniline (pNA) from the substrate H-D-Pro-Phe-Arg-pNA (S-2302). The rate at which the pNA is released was measured photo metrically at 405 nm.

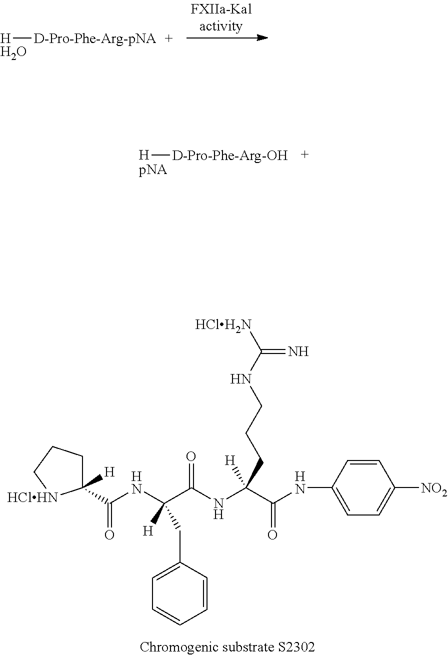

In summary, if the product is haemostatic it will lead to a color change and represent activation of the intrinsic pathway.

### Activated Partial Thromboplastin Times (APTTs)

The APTTs were determined using sample solution in HEPES buffer as agonists to activate the blood coagulation cascade when the intrinsic pathway was activated. Again HEPES buffer acted as a negative control and 1% m·doc powder made up in HEPES buffer acted as the positive control. The assays were carried out on a 96 well plate with the contents of each well outlined in Table 46.

TABLE 46
Contents of each well.

| | Neg. | Pos. | |
|---|---|---|---|
| | control | control | Sample |
| Solution | (μl) | (μl) | (μl) |
| Pooled normal plasma | 50 | 50 | 50 |
| Cephalin* | 50 | 50 | 50 |
| HEPES buffer | 50 | — | — |
| 1% m · doc solution | — | 50 | — |
| sample | — | — | 50 |
| Calcium chloride | 50 | 50 | 50 |

*Cephalin (1 μg/ml in TBS)

All the components (excluding the calcium chloride) were added to the wells and mixed once, then after a 10 minute wait the calcium chloride was added using a multichannel pipette directly before the experiment was carried out. The optical density change was recorded over 60 minutes at 405 nm using a microtitre platereader. Readings were taken every minute and samples were also agitated every minute. The assays were carried out in duplicate. To interpret results Microsoft excel was used, the optical density readings taken from each well every minute represent the absorbance of the contents of well. A curve was constructed of absorbance at 405 nm against time and the half max of the curve represents when the clot occurred. These values were used to construct a bar chart of clotting times for the agonists used. The max absorbance values represent the strength of the clot formed.

### Prothrombin Times (PTs)

These prothrombin times were determined using sample solution in HEPES buffer as agonists to activate the extrinsic pathway of the blood coagulation cascade. HEPES buffer acted as a negative control and 1% m·doc powder made up in HEPES buffer acted as the positive control. The assays were carried out in a 96 well plate with the contents of each well outlined in Table 47.

TABLE 47
Contents of each well

| | Control | Sample |
|---|---|---|
| Solution | (μl) | (μl) |
| Pooled normal plasma | 50 | 50 |
| Cephalin^{a} | 50 | 50 |
| HEPES buffer | 50 | — |
| sample | — | 50 |
| Thromboplastin^{b} | 50 | 50 |
| Calcium chloride | 50 | 50 |

^{a}Cephalin (1 μg/ml in TBS)
^{b}Thromboplastin was 1 μg/ml in TBS.

All reactants were mixed, added to the wells and allowed to incubate for 10 minutes. The calcium chloride was added last using a multichannel pipette directly before the optical density was read. The optical density change was recorded over 60 minutes at 405 nm using a microtitre platereader. Readings were taken every minute and samples were also agitated every minute. The assays were carried out in duplicate. To interpret results Microsoft Excel was used, the optical density readings taken from each well every minute represent the absorbance of the contents of well. A curve was constructed of absorbance at 405 nm against time and the half max of the curve represents when the clot occurred. These values were used to construct a bar chart of clotting times for the agonists used. The max absorbance values represent the strength of the clot formed.

### Platelet Aggregation Assay

Blood was drawn from healthy, non-aspirin-using volunteers using butterfly needles. Blood was anti-coagulated with sodium citrate (4%, 1:9, v/v) and centrifuged at 250×g for 10 minutes in capped plastic tubes. The straw colored upper phase was collected and this was the platelet rich plasma (PRP). Aggregation of platelets was performed in 96-well plates. 140 μl of PRP were pipetted per well in 96-well plate. The plate was pre-incubated at 37° C. for 3 minutes. The solution made from 2 mg samples in PBS were added to wells (10 μl) using a multichannel pipette and samples were agitated for 10 seconds. The optical density change was recorded over 30 minutes at 650 nm using a microtitre platereader. During the run the plate was incubated at 37° C. and readings were taken every 20 seconds and samples were also agitated 20 seconds.

### Coleman Whole Blood Clotting Test

Blood was drawn from healthy, non-aspirin-using volunteers using butterfly needles. Subsequently, 0.9 ml aliquots of the non-anti-coagulated blood was rapidly but gently, distributed into reagent-loaded (2 mg samples presoaked in deionized water) polystyrene tubes. There were three tubes for each determination. The tubes were tilted in a sequential manner and clot times were determined by the obvious formation of clots. The first tube was tilted at regular intervals until clotting was observed. Then the second tube was tilted until clotting was seen. This was repeated for the third tube and the clotting time of this tube was recorded as the clotting time of the hemostat. Clot times were expressed as the average of the three independent experiments and time zero was taken as the time the first tube was tilted.

### Results

The results are shown and discussed in FIGS. 86-93.

FIG. 86 compares the contact phase of the intrinsic pathway of various chitosan based materials, as well as listed in Table 48, below. Specifically, Kallikrien and factor XII activity in activated plasma was compared with one another. The data was the average of two separate experiments, and enzyme activity was expressed as mOD/min @ 405 nm. The results indicated that the electrospun materials, both the thermally treated and non-thermally treated materials had less enzyme activity than the chitosan-based HemCon bandage, which did not include electrospun materials.

TABLE 48
Samples in order of descending enzyme
activity values.

| Sample number | mOD/min |
|---|---|
| Sample 32 | 35.1 |
| HemCon bandage | 22.7 |
| Nano chitosan | 13.8 |
| (thermally treated) | |
| Nano chitosan (non- | 5.6 |
| thermally treated) | |

FIG. 87 demonstrates the intrinsic pathways of the compounds shown and discussed in FIG. 60, with the pathways plotted over time. The tests indicate that the thermally treated chitosan sample performed relative to the negative control, and the non-thermally treated chitosan sample performed relative to the HemCon bandage.

FIG. 88 provides clotting times (APTTs) of plasma for the tested compounds. The non-thermally treated chitosan material had a clotting time similar to the HemCon bandage, with the thermally treated material having a clotting time similar to that of the negative control.

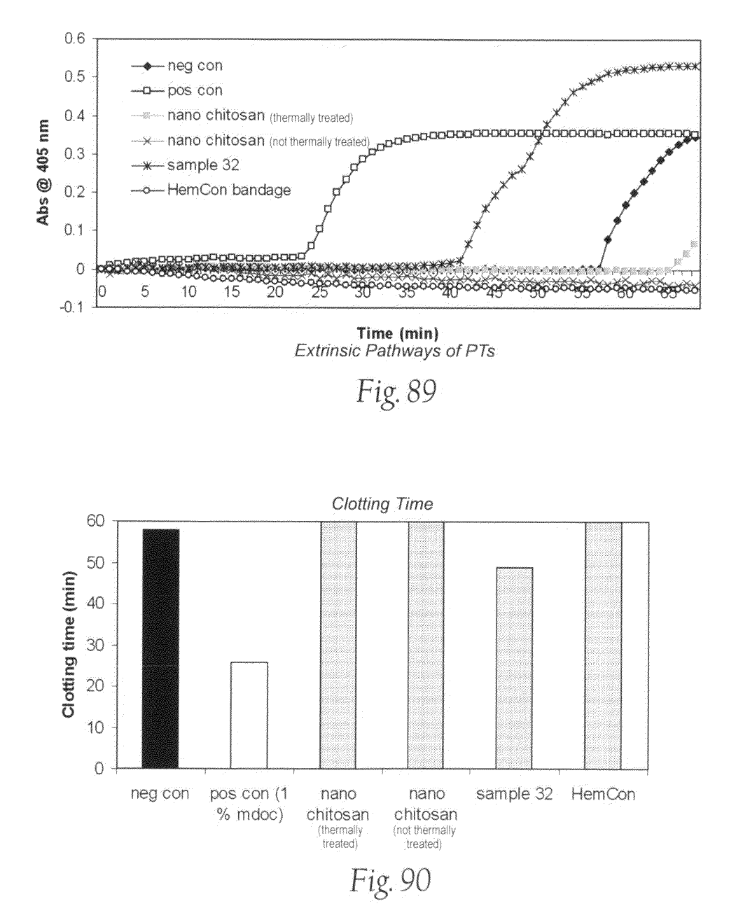

FIG. 89 shows the extrinsic pathway of PTs when the various samples were activated. As with the previous examples, the negative control represents APTT without an added hemostat, and the positive control represents APTT when a 1% MDOC solution was added to the sample. The clotting times for the samples under the same conditions as FIG. 89 are shown in FIG. 90. Clotting times and extrinsic pathways for both the thermally treated and non-thermally treated chitosan samples were similar to that of the HemCon bandage.

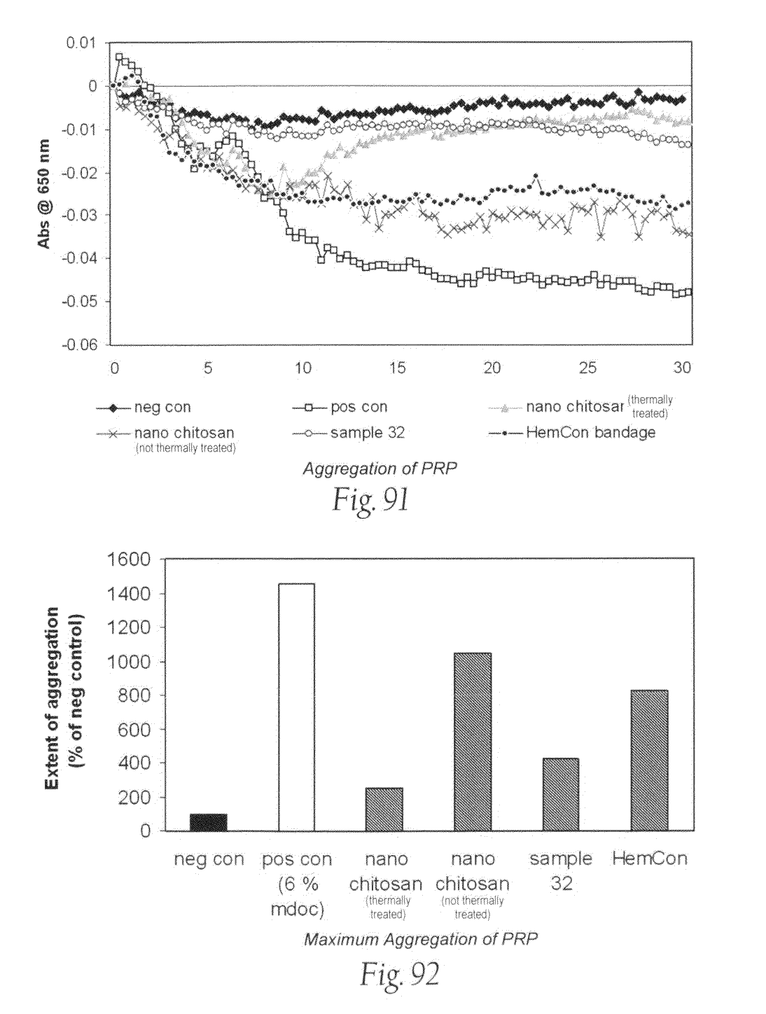

FIGS. 91-92 provide results of the aggregation of platelet rich plasma (PRP). The non-thermally treated chitosan sample appeared to have a much higher maximum decrease in absorbance than the crosslinked chitosan sample, compared to the negative control.

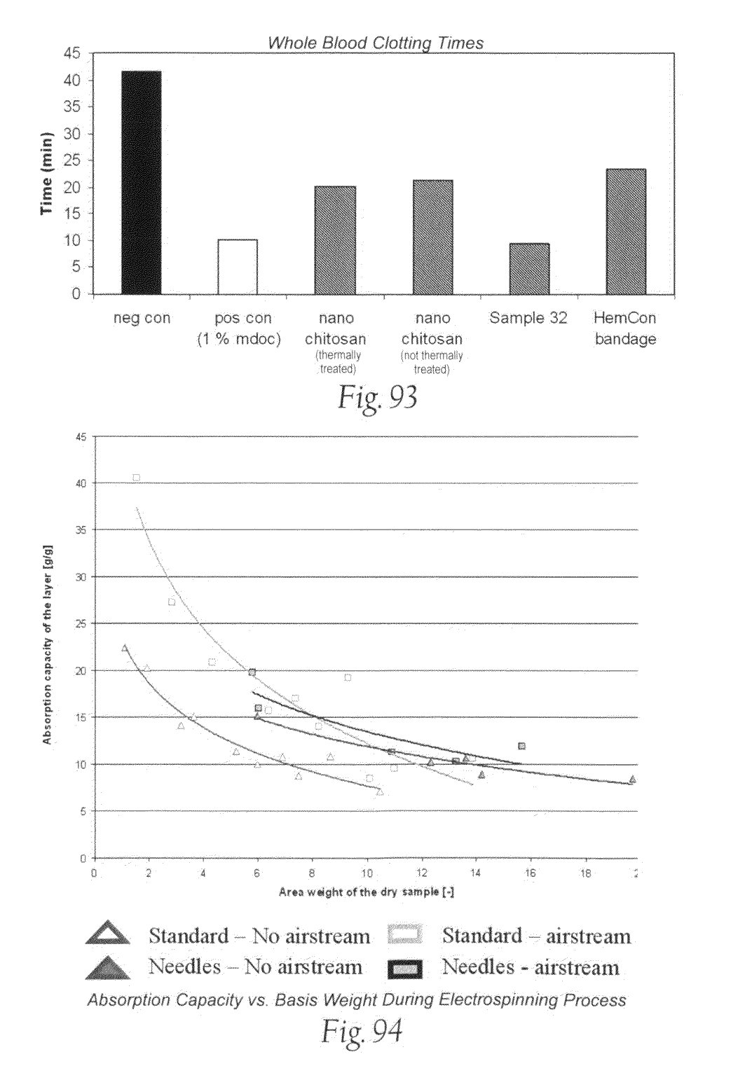

FIG. 93 compares the whole blood clotting times of the samples discussed, above, with the samples listed in Table 49, as well. The results indicate both the thermally treated and the non-thermally treated chitosan materials had clotting times similar to that of the HemCon bandage.

TABLE 49
Samples in order of descending
whole blood clotting time.

| Sample number | Time |
|---|---|
| Sample 32 | 9.4 |
| Nano chitosan | 20.1 |
| (thermally treated) | |
| Nano chitosan (non- | 21.3 |
| thermally treated) | |
| HemCon | 23.5 |

As is demonstrated from the above examples, electrospun nanomaterials provide for a wide range of therapeutic qualities, dependent on the chemical composition of the material that will be used in the electrospinning process. These qualities can be used as the basis for which materials may be used in an eventual wound dressing assembly. Depending on the particular requirement for a specific wound dressing, one or more of the various materials may be used in a wound dressing assembly.

Besides changes to the compositions of the electrospun materials, alterations to the parameters of the electrospinning process can also affect the properties of the resultant electrospun matrices. The following section discussed possible adaptations.

### IV. ELECTROSPINNING PARAMETERS

The electrospinning process was generally described with respect to FIG. 6, above. It has been noted that the quality of the resultant matrices of nanomaterials may be adjusted by adjusting various electrospinning parameters, such as the use of air streams during the electrospinning process, using different voltages for the collection of fibers, the process by which the fibers were collected (wire collection vs. needle collection).

A. Use of an Air Stream During the Electrospinning Process

A hot air stream (105° C.) can also be used as part of the electrospinning process. Depending on requirements the temperature of the air stream can be adjusted for a particular electrospinning process and it is used to increase the number of inter-fiber pores in the nanofiber matrix.

Matrices comprising chitosan and polyethylene oxide (PEO), as discussed above, were produced with and without the use of a hot air stream (105° C.) during the electrospinning process. The resultant Chitosan/PEO products were tested for absorption capabilities at a number of different densities. The same standard Chitosan product was also electrospun with the air stream described turned on. Also, the Chitosan nanofibers were electrospun and collected on a needle collection apparatus both with the air stream on and off. With the air stream off the process runs in an accumulation state with the humidity in the space climbing to some max level. An air stream would provide a steady state environment which will provide more consistent results from run to run. All four Chitosan/PEO samples were analyzed for their absorption capability and the results are presented in FIG. 94.

Absorption capacity decreased with increasing area weight. It was also clear that using the airstream approximately doubled the absorption capacity of the produced sample. It is assumed that these interfiber pores can hold and retain fluid better than the normally packed nanofiber matrix. Also, in case of spinning on needle apparatus the absorption capacity is higher when the air stream is used.

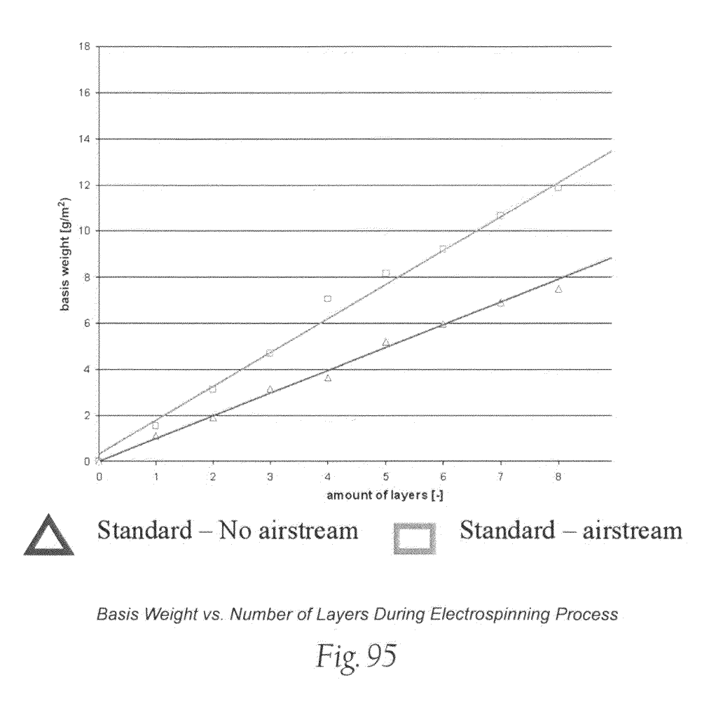

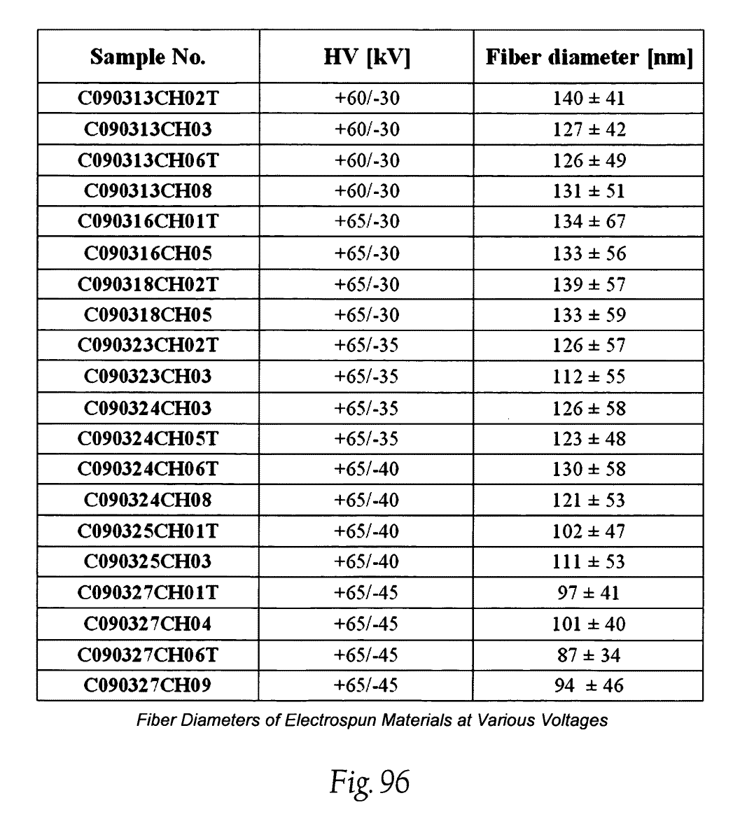

The effect on the basis weight of the produced samples with increasing nanofibers layers was also examined. Again this was performed both with and without the airstream. The results are presented in FIG. 95 and it can be seen that the dependence of area weight on the amount of layers is linear.

B. Change of Voltage Used During The Electrospinning Process

As discussed above with respect to FIG. 6, electrospinning processes are carried out by passing a material or solution between two electrodes connected in a circuit. The electrodes form an electric field with a voltage being provided across the electric field. Changes in the electric field may potentially have an effect on how the electrospun material is deposited during the electrospinning process. The following section discusses different applied voltages being used during the process.

Method

A solution containing 4.26 wt % dry matter, with a ratio of chitosan to polyethylene oxide (PEO) at a ratio of 93.6/6.4 w/w was tested to determine the effect of voltage change on the material. The solution had the parameters as listed in Table 50, below. “Fluffy” chitosan material

TABLE 50
Standard solution of a 3-D, i.e. “fluffy”
chitosan

| | | Weight | Dry | |
|---|---|---|---|---|
| | Weight (g) | (%) | matter | % |
| Solution 1 | | | | |
| Chitosan | 157.500 | 4.76 | 157.500 | 93.60 |
| demineralized | 1127.400 | 34.08 | | |
| water | | | | |
| Acetic acid | 2022.900 | 61.16 | | |
| 99% | | | | |
| Total weight | 3307.800 | 100.00 | | |
| Solution 2 | | | | |
| PEO | 10.764 | 3.00 | 10.764 | 6.40 |
| demineralized | 348.036 | 97.00 | | |
| water | | | | |
| Total weight | 358.800 | 100.00 | | |
| Final | 3666.600 | | 168.264 | 100.00 |
| solution | | | | |
| Chitosan | 157.500 | 4.30 | | |
| Demineralized | 1127.400 | 30.75 | | |
| water | | | | |
| Acetic acid | 2022.900 | 55.17 | | |
| 99% | | | | |
| PEO | 10.764 | 0.29 | | |
| Demineralized | 348.036 | 9.49 | | |
| water | | | | |
| | 3666.600 | 100.00 | | |

The electrospinning device had the following parameters.

### Electrospinning Device

Spinning electrode: smooth cylinder—35 cm length
Collector: cylinder
Electrode distance: 130 mm
Electrode spin: 4 rpm
Speed of the substrate: Initially 0.05/min, 2.5-3 cm/min during the spinning process

### HV Current: 90-115 kV

The solution was weighted before and after electrospinning process and an area of nanofiber sample was measured. The electrospinning process took approximately 24 minutes. Due to the high time consuming process, multiple solutions were prepared to carry out the testing and experiments, which had various viscosities and pH in dependence to temperature. For each combination of high voltage, a one- or two-cycled sample was tested and these samples were either in a treated (“crosslinked”) or non-treated (“non-crosslinked”) form. The one-cycle samples were processed using a new solution added to the reservoir of solution (see reference numeral 116, FIG. 6). During processing of the two-cycled samples, new solution to provide a consistent weight was added to the reservoir at the beginning of the process and was refilled as necessary for the second cycle to provide a full batch of solution.

Temperature was between 19.6-23° C. and the relative humidity was between 25.7-36% during the testing process. The following high voltages were applied during the testing process: +60/−30, +65/−30, +65/−35, +65/−40, +65/−45 and +65/−50 kV.

Properties of Solution and Nanomaterial

Conductivity, pH, viscosity, and temperature were measured for the tested solutions. There were some changes in viscosity due to different storage temperature. Generally speaking, conductivity and pH values of the solutions were affected by the storage time of the material prior the spinning process. Mixed solutions of chitosan and PEO that were mixed 24 hours prior to spinning and stored at room temperature did not appear to have the requisite conductivity for the spinning process. However, while mixing the chitosan/PEO solution approximately 30 minutes prior to the spinning process did provide the necessary conductivity and had an increased production capacity compared to the earlier prepared solutions, the homogeneity of the spun materials was lacking somewhat. When the storage temperatures of the 24 hour stored material was reduced (approximately to 14° C.), the solution was capable of being processed in the electrospinning process, providing a homogenous material, but with a lower production capacity It was determined that mixing the chitosan/PEO solution approximately 2 hours or so prior to spinning provided a balance between production capacity and homogeneity of the product.

Parameters were also measured for the nanofiber samples: basic area, fiber diameter, absorption capacity and area of nanofiber layer. Two absorption capacities were measured: immediately saturation of the sample (Acᵢ), and after 1 hour leaching in demineralized water when weight of sample was constant (Acₜ). This method includes absorption and swelling capability of the sample.

Parameters were also measured for the electrospinning process: consumption of solution after the electrospinning process (in g/min), various applied high voltages, relative humidity and temperature in laboratory.

FIG. 70 shows various samples that were spun at various currents, with corresponding fiber diameters listed.

### OBSERVATIONS AND CONCLUSIONS

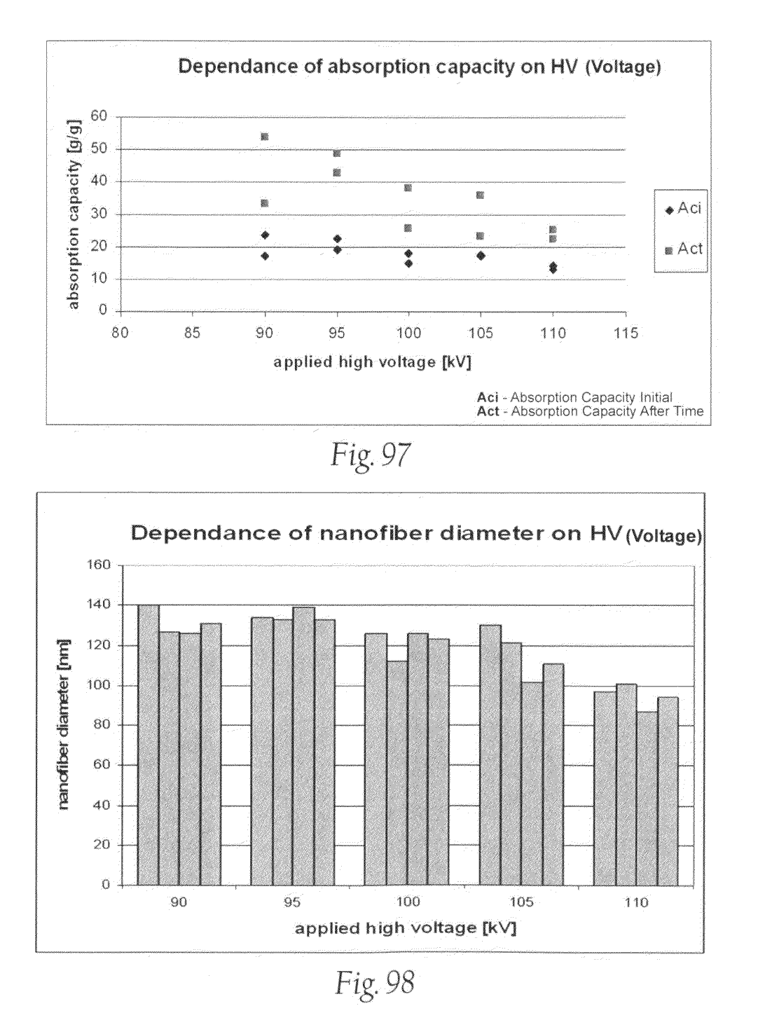

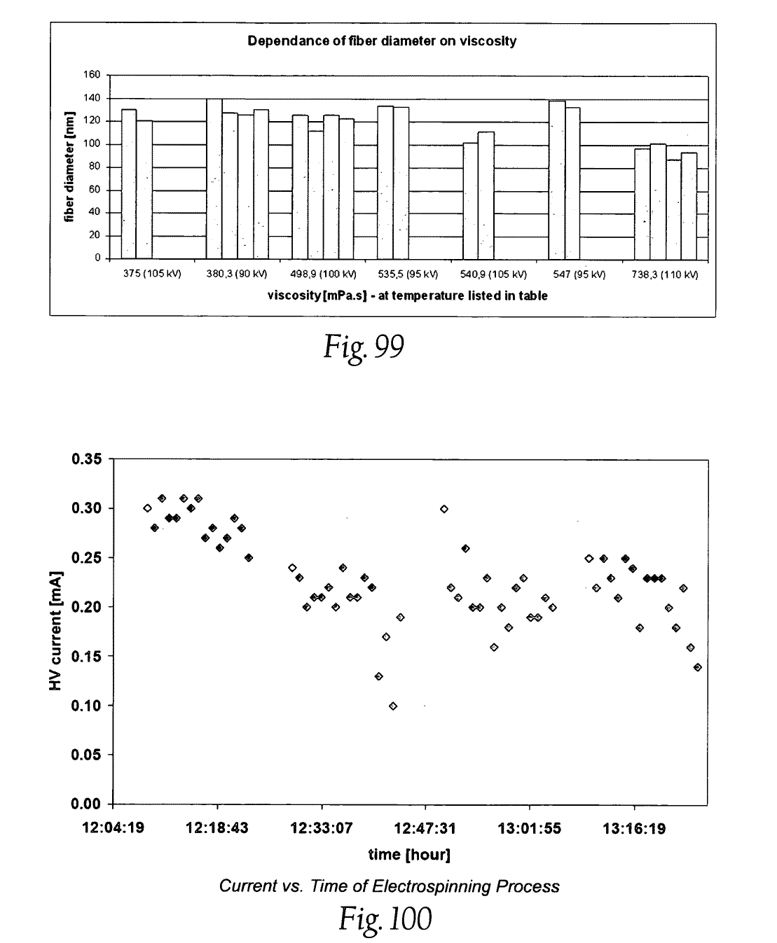

Generally, nanofiber diameter decreased with an increase in voltage, as shown in FIG. 97, and there is an increased production capability with an increased voltage. Initial results, while not conclusive, indicate that there may be a decrease in the absorption capacity (g/g) of the material as voltage increased, as shown in FIG. 98. This can be correlated with data shown in FIG. 99, which indicates that the fiber diameter may decrease with an increase in voltage. However, other factors, including the chemical makeup of the solution, as well as the relative mixing time for the solution with respect to the electrospinning process could affect production capacity. It was determined that using a voltage more than +65/−50 kV for the PP spunbond substrate was not possible, as it caused damage and burning to the base substrate.

As the results indicate, the “best combination” is a voltage of +60/−30 up to +65/−40 kV (90 kV to 115 kV). The highest absorption capability was achieved for +60/−30 kV, which was produced with a prepared polymer solution prepared approximately 2 hours before the electrospinning process. For all samples for +60/−30 kV, +65/−35 kV, the preparation time for the solution prior to electrospinning was approximately 2 hours. For the samples having currents of +65/−40 kV, solutions were prepared both at approximately 2 hours before electrospinning and at approximately 20-22 hours prior to electrospinning, with the latter being stored at approximately 14° C.

For the samples having currents of +65/−45 kV, the solution was mixed approximately 20-22 hours prior to electrospinning at a cool temperature (approximately 14° C.). The initial results indicated that the viscosity of the solution possibly increased, while production capability of the process, diameter of nanofibers and water absorption capability decreased. However, it should be understood that the samples were prepared differently, which makes it difficult to make a definitive correlation between the characteristics. That is, while initial indications tend to show a possible correlation, it may be that such a correlation is not prevalent.

The initial results indicate that with higher viscosity of the material, there may be a decrease in the diameter of the nanofibers, as shown in FIG. 99. However, it should be noted that the change in the fiber diameter could be as a result of the applied voltage, as shown in FIG. 100, or a combination of both. Likewise, as discussed above, preparation of the solutions may have an effect on such a potential correlation.

As such it can be seen that the voltage delivered during the electrospinning process has a potential effect on the produced materials. Depending on the eventual wound dressing assembly the material will eventually be used with, the voltage can be adjusted accordingly to change the absorption capacity or fiber diameter of the materials.

C. Different Electrospinning Methods

As previously discussed, there are alternate electrospinning processes or methods. Generally speaking, electrospinning methods relate to the way the nanomaterials and nanofibers are collected during the electrospinning process. The following section discusses alternate collection processes for the nanomaterials. The first spinning process is a wire collector process, with the spunbond moving passed the collecting electrode at the lowest fabric speed available (Spinning I). The second process alternatively used needle collection with a piece of spunbond material fixed on needles (Spinning II). Both processes were used in a dispersion system of MDOC in polyvinyl alcohol (PVA). The MDOC particles are dispersed in a continuous phase of the PVA solution.

### Spinning I

The dispersion mixture for spinning process I was a 16% m/m solution of MDOC and PVA, which was mixed in a ration of 4:6. The 16% solution (16±1.5%) was purchased from Novacke chemicke zavody, Slovakia (http://www.nchz.sk/?str=uovd&lang=en). The MDOC dispersion was prepared with 51.2 g of MDOC mixed with 270 g of deionized water using an UltraTurrax T25. The resulting dispersion was mixed with 479.9 g of the 16% solution and centrifuged for 3 minutes at 4000 rpm to remove any bubbles in the solution prior to electrospinning and testing of the viscosity measurements.

The system parameters were as follows:

Electrode: Big smooth cylinder

### Collector: Wire

### Electrode Distance: 130 mm

Fabric Speed: 0.13 m/min
Electrode spin 3.2 rpm

### HV Current: 82 kV

The dispersion spun for approximately 60 minutes, with a total dispersion mass of 800 g. The spinning time was approximately 1 hour, with samples of the materials taken at approximately every 15 minutes and the results were recorded. The following characteristics were noted:

### Temperature, Humidity, HV Current

Temperature and humidity of input and output air were monitored during 60 minutes of spinning using data loggers. HV current was recorded every 1 minute. The following results were noted.
Temperature changes of input air within 0.4° C. during the spinning process.
Relative humidity of input air decreases by about 5% during the spinning process.
Temperature of output air is higher than input air by about 1.8° C., changes within 0.4° C. during the spinning process.
Relative humidity of output air is lower than that of input air by about 0.5-1.7% and decreases by about 5% during the spinning process.
Absolute humidity of output air is higher than that of input air by about 0.3-0.7 g/m³. Absolute amount of water evaporated decreases with the spinning process evolution.
HV current decreased from the start of the spinning process. See FIG. 100.

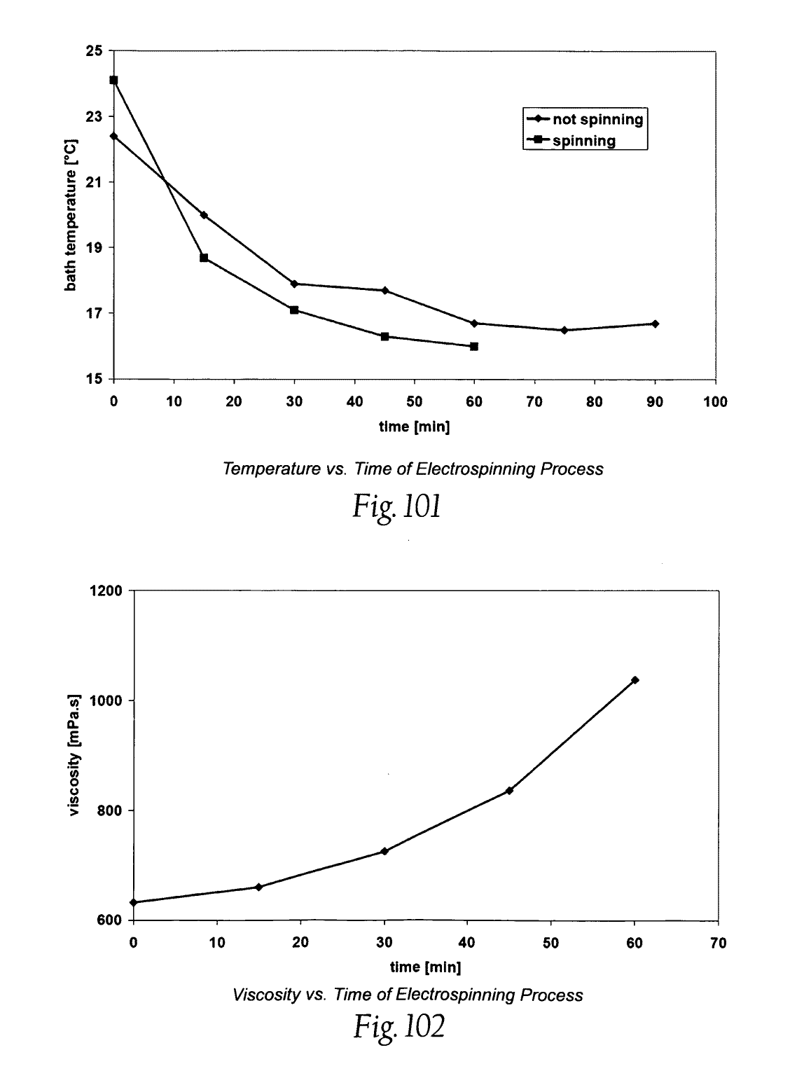

Temperature of dispersion in bath was measured at 0, 15, 30, 45 and 60 min.
Temperature of dispersion in bath decreased from 24.1° C. to 16.0° C. during 60 minutes of spinning. See. FIG. 98.
Temperature of dispersion in bath decreased from 22.4 to 16.7° C. during 60 minutes without spinning. FIG. 101.

### Properties of Dispersion and Nanomaterial

Parameters measured for dispersion: viscosity, loss on drying/dry matter; conductivity
Dry matter and conductivity increased by 4% during 60 min of spinning.
Viscosity increased by 65% during 60 min of spinning. FIG. 102.

Parameters measured for nanospun material: only a thin layer of nanomaterial was spun onto spunbond; hence, only approximate values of area weight are presented. Consequently loss on drying/dry matter was not determined.
Nanofiber area weight decreased from approximately 3 g/cm² to approximately 2 g/cm² during the spinning process.

### Spinning II

The dispersion used for the Spinning II process was the same as describe above for the Spinning I process.

The system parameters were as follows:

Electrode: Big smooth cylinder
Collector: Needles with Spunbond Fixed

### Electrode Distance: 130 mm

### Fabric Speed: N/A

Electrode spin 3.2 rpm

### HV Current: 60 kV

The MDOC:PVA dispersion was spun 4 times for 15 minutes. The nanospun material and spunbond was removed after spinning and replaced by new piece of spunbond. The following characteristics were noted:

Temperature, humidity, HV current

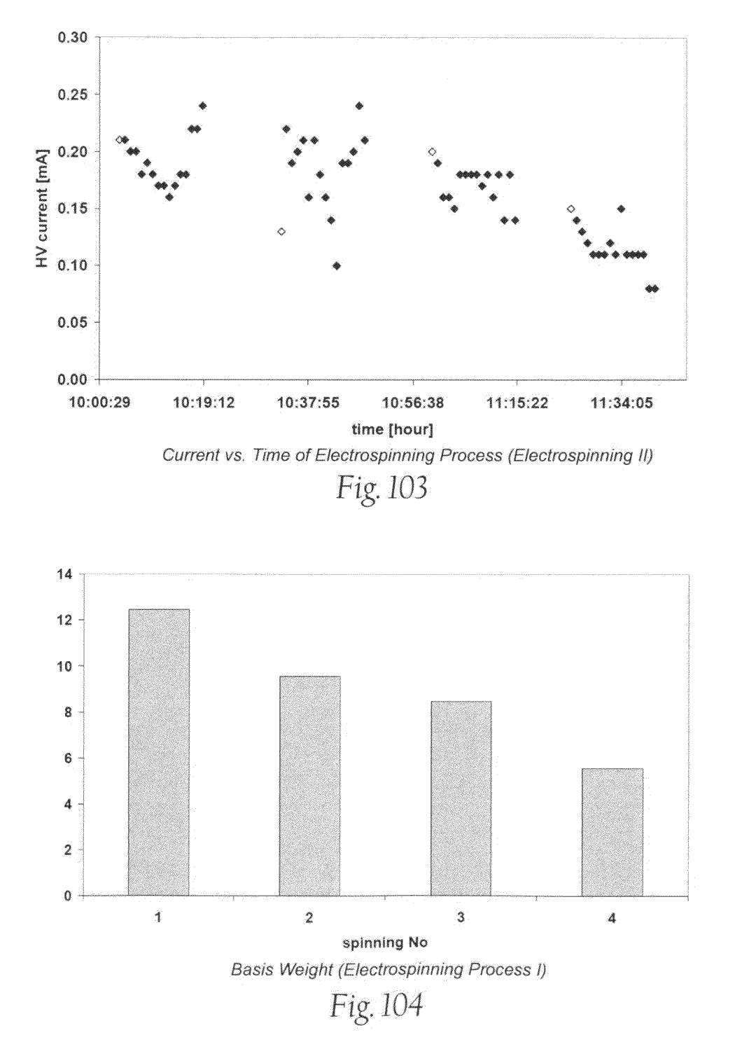

Temperature and humidity of input air were monitored during spinning using data loggers. HV current was recorded every 1 minute. The following characteristics were noted:
Temperature changes of input air within 0.4° C. during spinning.
Relative humidity of input air decreases by about 2% during spinning.
HV current decreased from the start of spinning. FIG. 103.

### Properties of Nanomaterial

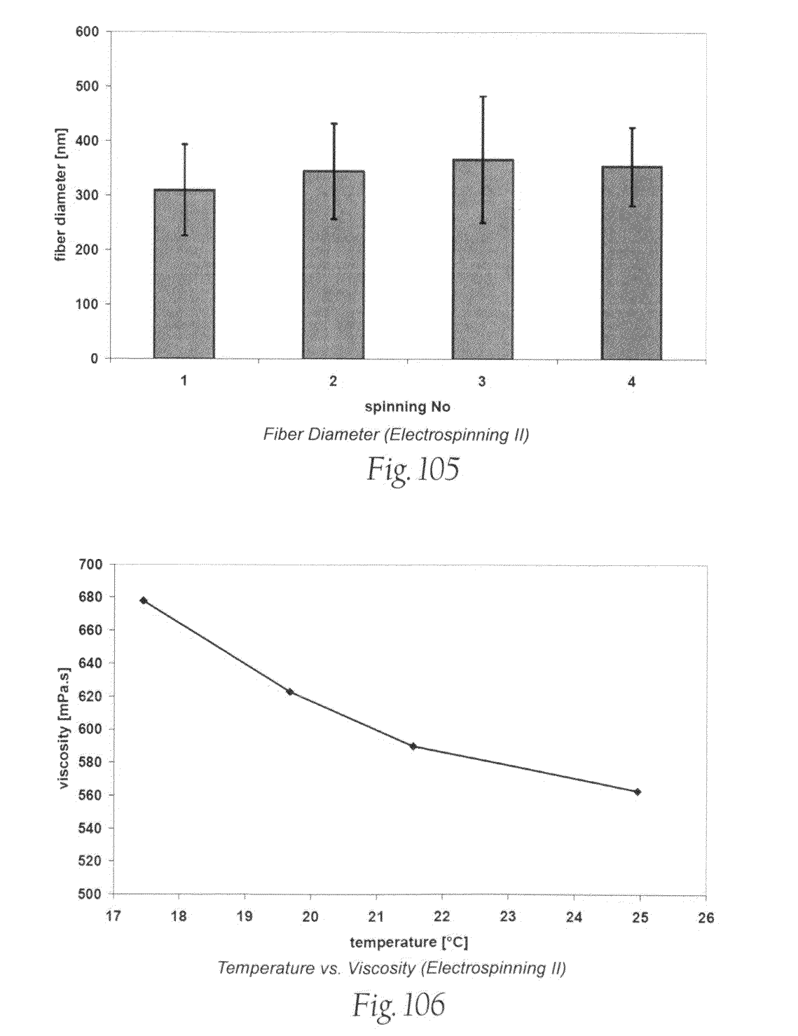

Parameters measured for nanospun material: area weight, loss on drying/dry matter, fiber diameter (Scanning electron microscope image provided using JEOL 6490 LV, microscope in cooperation with Laboratory of electron microprobe and microscopy, Department of Geological Sciences, Faculty of Science, Masaryk University, Brno, CZ).
Nanofiber area weight decreased from 12.5 to 5.5 g/cm² during the spinning process. FIG. 104.
Nanofiber dry matter changes within 1% (i.e. 91-92%, with no indicated trend)
No significant change in fiber diameter, as demonstrated in FIG. 105.

### The Effect of Temperature or Dispersion Concentration on Viscosity

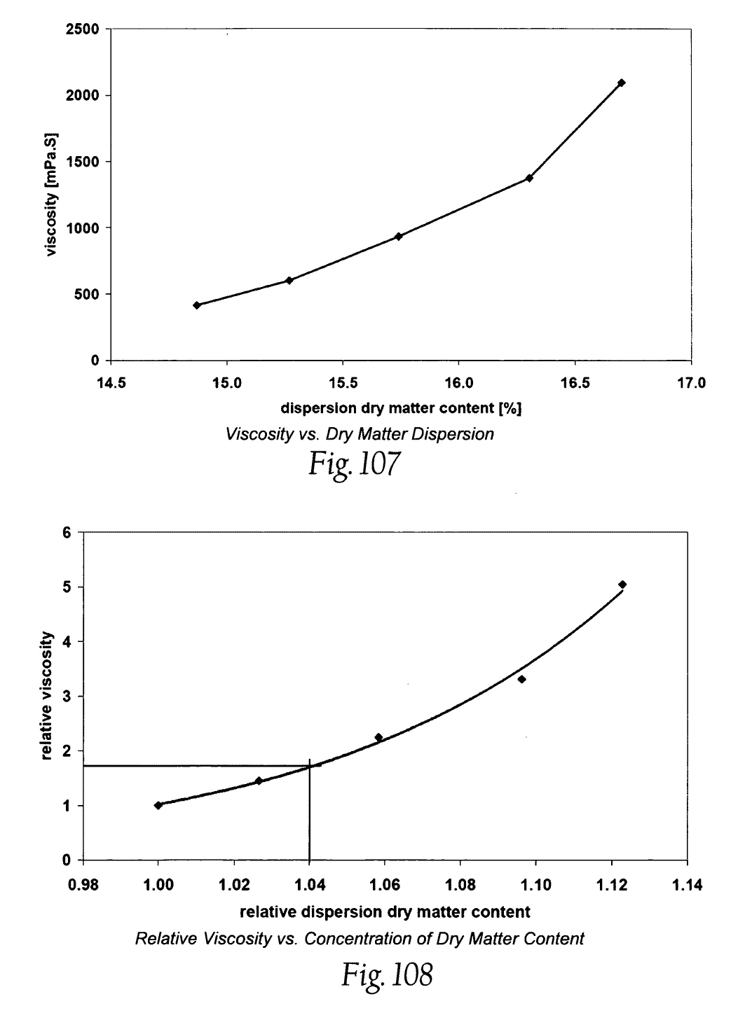

The effect of temperature was examined in range 17-25° C. The effect of dry matter in dispersion was_examined in range 14.9-16.7%. The lowest value (14.9%) corresponds to a regular MDOC:PVA dispersion. Higher concentration of polymers in dispersion was provided by partial evaporation of water from regular dispersion using a rotary vacuum evaporator. Dry matter of dispersion was determined experimentally. The following characteristics were noted:
Viscosity increases by 20% when temperature decreases from 25 to 17° C. FIG. 106.
Viscosity increases by about 60-70% when dry matter of dispersion increases by 4% (i.e. dispersion after 60 minutes of spinning). See FIGS. 107 and 108.

### Further Observations

Temperature of dispersion bath decreases as a result of solvent evaporation even when the spinning is switched off. Contribution of spinning process itself to the temperature decrease has a minor effect.

Concentration of polymer(s) in dispersion increases during spinning due to solvent evaporation. Viscosity of dispersion increases as a result of combination of both effects (increasing concentration and drop in temperature). The effect of increasing concentration seems to be more crucial; however, the exact role of both effects simultaneously would have to be investigated in more details to get conclusive results.

Decreasing area weight of nanomaterial can be observed as a result of drop in electrospinning efficacy in time. SEM analysis did not reveal significant differences of nanofiber diameter or morphology during spinning.

The following is a simple scheme of observed effects:

Evaporation of solvent (water)→temperature drop & thickening of dispersion due to evaporation→viscosity increase→decrease of electrospinning efficacy & HV current→decrease in nanomaterial area weight.

### V. CONCLUSION

Wound dressing assemblies according to the present invention may comprise a wide range of materials, having a wide range of therapeutic and hemostatic properties. The wound dressing assemblies may comprise multiple layers of materials, with each layer of material providing a different quality for the overall wound dressing assembly. Once it is determined what specific qualities are to be used in a wound dressing assembly, the present invention provides guidance in determining individual layers for a dressing assembly, as well as the ability to the electrospinning process to further provide materials having various characteristics.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A method for producing a wound dressing assembly comprising:
providing a plurality of individual layers of electrospun nanomaterials; and
adhering said layers together to form said wound dressing.

2. The method according to claim 1, wherein said individual layers of electrospun materials are adhered together after being electrospun.

3. The method according to clam 1, wherein the individual layers are adhered to one another during the electrospinning process.

4. The method according to claim 3, wherein the individual layers are adhered to one another during the electrospinning process.

5. The method according to claim 4, wherein the individual layers are adhered to one another using a low temperature melt material.

6. The method according to claim 5, wherein the low temperature melt material is an electrospun polyhydroxy butyrate material.

7. The method according to claim 1, wherein said individual layers of materials are selected from the group consisting of: chitosan compounds, alginate compounds, cellulose compounds, including oxidized cellulose compounds, mircodispersed oxidized cellulose compounds, carboxymethyl cellulose compounds, hydroxyethlyl, cellulose compounds, methyle cellulose compounds, and ethyle cellulose compounds collagen compounds, fibrinogen compounds, albumen compounds, cellulose acetate compounds, nylon compounds, polyurethane compounds, polyurethane ester, ether, urea, and siloxane compounds, polyactic acid compounds, polyglycolic acid compounds, polyhydroxybutyric acid compounds, polyglycoliclactic acid compounds, polyvinyl alcohol and alcohol compounds, polyethylene oxide compounds, gelatin compounds, hyaluronic acid compounds, polycaprolactone compounds, silk compounds, pectin compounds, polyacrylic acid compounds, starch compounds, and combinations thereof.

8. The method according to claim 1, wherein the electrospinning process is carried out with a needleless electrospinning process.

9. A method for producing a wound dressing assembly comprising:
determining the treatment to be addressed by the wound dressing assembly;
determining the requirements for the wound dressing assembly when carrying out the treatment of the wound dressing assembly;
determining the materials capable of carrying out the treatments and requirements of the wound dressing assembly;
producing individual layers of electrospun materials based on the determined treatments, requirements, and materials for the wound dressing assembly; and
forming said wound dressing assembly from said individual layers of electrospun materials.

10. The method according to claim 9, wherein at least one of said individual layers of materials is selected from the group consisting of: chitosan compounds, alginate compounds, cellulose compounds, including oxidized cellulose compounds, mircodispersed oxidized cellulose compounds, carboxymethyl cellulose compounds, hydroxyethlyl, cellulose compounds, methyle cellulose compounds, and ethyle cellulose compounds collagen compounds, fibrinogen compounds, albumen compounds, cellulose acetate compounds, nylon compounds, polyurethane compounds, polyurethane ester, ether, urea, and siloxane compounds, polyactic acid compounds, polyglycolic acid compounds, polyhydroxybutyric acid compounds, polyglycoliclactic acid compounds, polyvinyl alcohol and alcohol compounds, polyethylene oxide compounds, gelatin compounds, hyaluronic acid compounds, polycaprolactone compounds, silk compounds, pectin compounds, polyacrylic acid compounds, starch compounds, and combinations thereof.

11. The method according to claim 8, wherein said process is carried out with a needleless electrospinning process.

12. A wound dressing assembly according to the process of claim 8.

13. A wound dressing assembly comprised of a plurality of individual layers of electrospun nanomaterials.

14. The wound dressing assembly of claim 13, wherein at least one of the individual layers is selected from the group consisting of: chitosan compounds, alginate compounds, cellulose compounds, including oxidized cellulose compounds, mircodispersed oxidized cellulose compounds, carboxymethyl cellulose compounds, hydroxyethlyl, cellulose compounds, methyle cellulose compounds, and ethyle cellulose compounds collagen compounds, fibrinogen compounds, albumen compounds, cellulose acetate compounds, nylon compounds, polyurethane compounds, polyurethane ester, ether, urea, and siloxane compounds, polyactic acid compounds, polyglycolic acid compounds, polyhydroxybutyric acid compounds, polyglycoliclactic acid compounds, polyvinyl alcohol and alcohol compounds, polyethylene oxide compounds, gelatin compounds, hyaluronic acid compounds, polycaprolactone compounds, silk compounds, pectin compounds, polyacrylic acid compounds, starch compounds, and combinations thereof.

15. The wound dressing assembly of claim 13 wherein at least one of the individual layers comprises a chitosan compound.

16. The wound dressing assembly according to claim 15, wherein said chitosan compound has been treated with an acid.

17. The wound dressing assembly of claim 15, wherein said chitosan compound comprises a chitosan/polyethylene oxide (PEO) compound.

18. The wound dressing assembly of claim 17 wherein said chitosan/PEO compound has been treated with acetic or lactic acid.

19. The wound dressing assembly of claim 15, wherein said chitosan compound comprises a chitosan/polyvinyl alcohol (PVA) compound.

20. The wound dressing assembly of claim 19, wherein said chitosan/PVA compound has been treated with acetic acid or lactic acid.

21. The wound dressing assembly of claim 15, wherein said chitosan compound comprises chitosan/PEO or chitosan/PVA or combinations thereof.

22. The wound dressing assembly of claim 15, wherein said chitosan/PEO compound further comprises a silver compound.

23. The wound dressing assembly of claim 15, wherein at least one of the individual layers comprises a microdispersed oxidized cellulose (MDOC) compound.

24. The wound dressing assembly of claim 23, wherein said MDOC compound further comprises a polyvinyl alcohol (PVA) compound.

25. The wound dressing assembly of claim 23 wherein said MDOC/PVA compound further comprises an MDOC/PVA/PEO compound.

26. The wound dressing assembly of claim 15, wherein at least one of the individual layers comprises a gelatin compound.

27. The wound dressing assembly according to claim 26 wherein the gelatin compound further comprises at least one other compound selected from the group consisting of: PEO compounds, PVA compounds, MDOC compounds, MDOC H+ compounds, and carboxymethylcellulose (CMC) compounds.

28. The wound dressing assembly according to claim 15, wherein at least one of the individual layers of material comprises a CMC/PVA/PEO compound.

29. The wound dressing assembly according to claim 28, wherein said CMC/PVA/PEO compound further comprises a silver compound.

30. The wound dressing assembly of claim 15, further comprising a backing layer of nonspun materials adjoined to said layers of electrospun materials.

31. A needleless method of forming an electrospun wound dressing assembly comprising:
providing a first solution for forming an electrospun layer of material;
providing an adhesive material to form an electrospun layer;
providing a second solution for forming a second electrospun material;
concurrently electrospinning said first solution, said adhesive material, and said second solutions; and
forming a wound dressing assembly wherein said first electrospun material is adhered to said second electrospun material.

32. The method according to claim 31, wherein said adhesive material is a low temperature melt material.

33. The method according to claim 32, wherein the low temperature melt material is an electrospun polyhydroxy butyrate material.

34. The method according to claim 31 wherein at least one of the solutions is selected from the group consisting of: chitosan compounds, alginate compounds, cellulose compounds, including oxidized cellulose compounds, mircodispersed oxidized cellulose compounds, carboxymethyl cellulose compounds, hydroxyethlyl, cellulose compounds, methyle cellulose compounds, and ethyle cellulose compounds collagen compounds, fibrinogen compounds, albumen compounds, cellulose acetate compounds, nylon compounds, polyurethane compounds, polyurethane ester, ether; urea, and siloxane compounds, polyactic acid compounds, polyglycolic acid compounds, polyhydroxybutyric acid compounds, polyglycoliclactic acid compounds, polyvinyl alcohol and alcohol compounds, polyethylene oxide compounds, gelatin compounds, hyaluronic acid compounds, polycaprolactone compounds, silk compounds, pectin compounds, polyacrylic acid compounds, starch compounds, and combinations thereof.
